SEQUENCE LISTING (2 sequences)

SEQ ID NO: 1 - DNA, 24 bp, Artificial Sequence
  Synthetic polynucleotide
agaaacccttcagtgggacttaac

SEQ ID NO: 2 - DNA, 24 bp, Artificial Sequence
  Synthetic polynucleotide
cggaatttagaaacccggctccac

(19) United States
(12) Patent Application Publication
(10) Pub. No.: US 2021/0169883 A1
(43) Pub. Date: Jun. 10, 2021

(54) METHOD OF TREATING AGGRESSION WITH OREXIN RECEPTOR ANTAGONISTS

(71) Applicant: Icahn School of Medicine at Mount Sinai, New York, NY (US)

(72) Inventors: Meghan Flanigan, Chapel Hill, NC (US); Scott Russo, Pelham Manor, NY (US)

(73) Assignee: Icahn School of Medicine at Mount Sinai, New York, NY (US)

(21) Appl. No.: 17/263,488

(22) PCT Filed: Jul. 25, 2019
(86) PCT No.: PCT/US2019/043394

§ 371 Date: Jan. 26, 2021

Related U.S. Application Data
(60) Provisional application No. 62/711,233, filed on Jul. 27, 2018.

(51) Int. Cl. A61K 31/506; A61K 31/551; A61K 31/472; A61K 31/444; A61K 31/36; A61K 9/00; A61P 25/00

(52) U.S. Cl. CPC A61K 31/506; A61K 31/551; A61K 31/472; A61P 25/00; A61K 31/36; A61K 9/0056; A61K 31/444

(57) ABSTRACT

The present invention provides orexin receptor antagonists for use in treating aggression in a subject. The orexin receptor antagonists can have selective affinity for orexin receptor 1 (Ox1R) or for orexin receptor 2 (Ox2). The orexin receptor antagonists can also have dual affinity for both Ox1R and Ox2R, wherein their affinities can be the same or different. The present invention also provides pharmaceutical compositions and kits comprising orexin receptor antagonists for treating aggression or for treating psychiatric disorders with an aggression component.

### RELATED APPLICATIONS

This application claims priority to U.S. Provisional Patent Application No. 62/711,233, filed Jul. 27, 2018, the entire contents of which are incorporated herein by reference.

### FEDERALLY SPONSORED RESEARCH

This invention was made with Government support under Grant No. R01MH114882 awarded by the National Institute of Mental Health. The Government has certain rights in the invention.

### FIELD OF THE DISCLOSURE

The present invention relates to the clinical treatment of aggression or aggressive behavior. In particular, the present invention relates to methods of treating subjects having a range of psychiatric disorders that have associated aggression or aggressive behavior symptoms. More in particular, the present invention relates to the use of orexin receptor antagonists to treat aggression or aggressive behavior in a subject, while minimizing or avoiding undesirable side-effects that include effects on the sleep/wake cycle and/or cognition.

### BACKGROUND

Interpersonal aggression and violence is a prevalent symptom of many neuropsychiatric disorders including but not limited to drug addiction (Beck et al., 2014; Coccaro et al., 2016), autism (Fitzpatrick et al., 2016), depression (Dolenc et al., 2015), antisocial personality disorder (Anderson and Kiehl, 2014), PTSD (Miles et al., 2016), and schizophrenia (Hoptman, 2015). In addition to producing a variety of negative social and physical consequences for victims and patients, those who experience or witness violence display increased risk for developing neuropsychiatric disorders like PTSD and depression (Sumner et al., 2015).

While clearly a tremendous public health issue, there are currently no approved treatments aimed at reducing aggression in psychiatric patients. Selective serotonin reuptake inhibitors, benzodiazepenes, and anti-psychotics are widely prescribed off-label to treat aggression, but the efficacy of these drugs is extremely limited and they can exhibit a number of undesirable side effects (Goedhard et al., 2006).

Recent evidence in humans and animal models suggests that aggression is highly reinforcing, and aggression in psychiatric patients is associated with structural and functional abnormalities in reward-related brain regions like the nucleus accumbens (NAc) (Couppis and Kennedy, 2008; Decety et al., 2009; Cha et al., 2015; Golden et al., 2016b; Golden et al., 2017). Treatments aimed at reducing the positive valence of aggression through modulation of reward circuitry may be promising options in clinical populations for which elevated aggression is a symptom. The hypothalamic neuropeptide orexin (hypocretin) has been shown to exert modulatory control over reward-related nuclei to critically influence motivation in behavioral paradigms related to feeding, social interaction, and drug seeking (Sakurai, 2014). It is therefore possible but not previously known that the brain's orexin signaling system may also influence motivation for engaging in aggression.

The orexin signaling system is a highly evolutionary conserved neuropeptide-G-protein-coupled receptor (GPCR) system first discovered in 1998 (de Lecea et al., Proc Natl, Acad Sci USA, 1998, 95:322-327 and Sakuri et al., Cell, 1998, 92:1). The system comprises two receptors, the orexin 1 receptor (Ox1R) and orexin 2 receptor (Ox2R), and two neuropeptides, namely, orexin A (OxA) and orexin B (OxB). OxA is a 33-amino acid peptide that binds with similar affinity to Ox1R and Ox2R, whereas OxB is a 28-amino acid peptide exhibits a much higher selective affinity for Ox2R. Both of these peptides, while relatively few in number, are produced by excitatory neurons in the lateral hypothalamus (LH) (in the perifornical area) by a cascade of enzymatic reactions from the precursor, pre-pro-orexin. Orexin-producing cells in these regions project to many areas of the brain, extending to the olfactory bulbs and to the spinal cord (van den Pol, A. N. et al., J. Neuroscience., 1999, 19(8), 3171-3182). This broad distribution of orexin projections and neurons expressing orexin receptors within the central nervous system is suggestive of orexin involvement in a number of physiological functions including feeding, drinking, arousal, stress, reward, metabolism and reproduction (T. Sakurai, Nature Reviews Neuroscience, 2007, 8(3), 171-181).

For example, orexin signaling system is thought to be a key player in the regulation of sleep and wakefulness and promotes the appropriate arousal level to adapt to the environmental conditions (Sakurai et al., Nat Rev Neurosci., 2007; 8:171-181; Li S B et al., Curr Top Behav Neurosci. 2017; 33:93-104, and Mahler et al., Nat Neurosci. 2014; 17:1298-1303). It has been reported that the disruption of the orexin signaling system results in type 1 narcolepsy. The orexin signaling system also plays a key role in the physiological and behavioral responses to external environmental stimuli, and dysregulation of the system can lead to anxiety, stress, and addiction (James et al., Curr Top Behav., Neurosci. 2017; 33:197-219; Aston-Jones et al., Brain Res. 2010; 1314:74-90; James et al., Curr Top Behav Neurosci., 2017; 33:247-281). Moreover, the orexin system has been associated with panic/anxiety and depression. The orexin system is also regarded as integrator and coordinator of physiological and behavioral responses, e.g., the translation of hunger (which activates orexin neurons) into appetite and to trigger the motivation to seek food (So et al., J Physiol Sci. 2017). In addition, the orexin system is involved in the regulation of cognition and motor activity and has a role in influencing cardiovascular, metabolic, respiratory, or homeostatic processes. In addition, the orexin signaling system is thought to be associated with a number of disorders, including Alzheimer's disease (Kang et al, Science Express, 2009, 1-10), drug and alcohol addiction (G. Aston-Jones et al., Neuropharmacology, 2009, 56 (Suppl 1) 112-121), and overweight or obesity and conditions related to overweight or obesity, such as insulin resistance, type II diabetes, hyperlipidemia, gallstones, angina, hypertension, breathlessness, tachycardia, infertility, sleep apnea, back and joint pain, varicose veins and osteoarthritis.

The identification of orexin receptor modulators remain a strong continued interest in the art and an active area of research as they represent potential therapeutic agents that may treat a wide variety of disorders mediated through this signaling system. To date, the development of orexin receptor antagonists has largely focused on treating sleep conditions. For example, suvorexant (marketed by Merck & Co. as BELSOMRA® since 2015), is a selective, dual orexin receptor antagonist for treating insomnia. Development of orexin receptor antagonists for treating Alzheimer's disease and substance addiction are also underway.

However, prior to the present invention, the role of the orexin signaling system in aggression or in psychiatric disorders having an aggression component was not recognized or known. The present invention demonstrates for the first time that the orexin signaling system has a role in regulating aggression and further that the orexin signaling system can be therapeutically targeted with orexin receptor antagonists to clinically treat aggression. Thus, the invention meets an unmet medical need to provide effective treatments for aggression where currently there are none or very few.

### SUMMARY

The present invention relates in part to the surprising discovery that the orexin signaling system has a role in regulating and/or controlling aggression and aggressive behaviors. It is believed that this realization was not previously known. Thus, the orexin signaling system represents a new and useful therapeutic target for treating aggression and aggressive behaviors. Accordingly, in one aspect, the present invention relates to orexin receptor antagonists for use in treating aggression. In another aspect, the present invention relates to pharmaceutical compositions comprising orexin receptor antagonists for treating aggression. In still other aspects, the present invention relates to methods for treating aggression, or treating psychiatric disorders with an aggression component, by administering an effective amount of an orexin receptor antagonist, or an effective amount of a pharmaceutical composition comprising an orexin receptor antagonist. In some aspects, the orexin receptor antagonists have selective affinity for orexin receptor 1 (Ox1R). In other aspects, the orexin receptor antagonists have selective affinity for orexin receptor 2 (Ox2). In still other aspects, the orexin receptor antagonists have affinity for both Ox1R and Ox2R, wherein their affinities can be the same or different. In still other aspects, the present invention provide pharmaceutical kits comprising a pharmaceutical composition comprising an orexin receptor antagonist and a set of instructions for administering the composition. In still other aspects, the invention may involve the co-administration of an orexin receptor antagonist (or a composition comprising same) and another therapeutic.

The present summary and the following detailed description of the present invention are exemplary only and are not intended to be restrictive of the disclosure as a whole. Other aspects of the present disclosure not expressly described herein will be apparent to those skilled in the art in view of the detailed description and disclosure provided herein.

Several exemplary embodiments are as follows.

In one embodiment, the specification provides a method for treating aggression in a subject comprising administering a therapeutically effective amount of compound I:

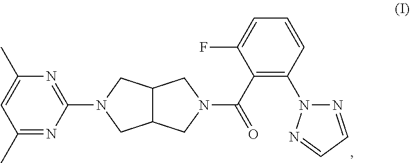 [SMILES: Cc1cc(C)nc(N2CC3CN(C(=O)c4c(F)cccc4-n4nccn4)CC3C2)n1]

or a pharmaceutically acceptable salt thereof.

In another embodiment, the specification provides a method for treating aggression comprising identifying a subject having aggression, and inhibiting the OX2 receptor.

In yet another embodiment, the specification provides a method for treating aggression comprising identifying a subject having aggression, and administering a therapeutically effective amount of an OX2 receptor antagonist without having a sedative effect on the subject as compared to a control subject.

The specification also provides in another embodiment a method of treating an animal having aggressive, comprising administering to an animal a therapeutically effective amount of an OX2 receptor antagonist without having a sedative effect on the animal as compared to a control subject.

In another embodiment, the specification provides a method for reducing aggression produced as a side-effect of an agent administered to a subject, comprising identifying a subject displaying aggression induced by the agent, and administering a therapeutically effective amount of an OX2 receptor antagonist.

In various embodiments, the aggression that is being treated can arise in association with or as a symptom of one or more neurological and psychiatric diseases or disorders, which can include, Intermittent Explosive Disorder, Kleefstra Syndrome, Impulsive Aggression, X-Linked Intellectual Disability-Hypotonia-Facial Dysmorphism-Aggressive Behavior Syndrome, Gaucher Disease Type 3, Hunters Syndrome (MPS II), and Drug-Induced Aggression.

In various other embodiments, the aggression that is being treated can arise in association with or as a symptom of excessive alcohol use, anabolic steroids, benzodiazepines, or cocaine, i.e., drug-induced aggression.

The methods can be used to treat mammals. The mammals can be animals, such as cats, dogs, horses, or other livestock. The mammals can also be human subjects.

In preferred embodiments, the compounds of the invention are administered in a manner that does not simultaneously cause sedation or have a cognitive effect compared to a control subject.

In various embodiments, the administration of the compounds can be by oral, intranasal, transdermal, or intravenous routes. The compounds can be formulated as pharmaceutical compositions suitable for delivery by these routes.

In various embodiments, the small molecule compound delivered by the disclosed methods is selected from the group consisting of: suvorexant, almorexant, seltorexant, EMPA, filorexant, JNJ-10397049, and lemborexant.

In still other embodiments, the invention provides a method for reducing aggressive behavior in a human subject having a mental health disorder, comprising identifying a subject having a mental health disorder who displays an aggressive behavior, and administering a therapeutically effective amount of an OX2 receptor antagonist. The mental health disorder can be selected from the group consisting of: Alzheimer's disease, attention-deficit/hyperactivity disorder (ADHD), autism spectrum disorder, bipolar disorder, dementia, schizophrenia, and Huntington's disease.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1J. In vivo monitoring of LHb (lateral habenula) activity during social encounters in the RI test. FIG. 1A, Experimental scheme and viral expression, scale bar=100 μm. FIG. 1B, LHb GCaMP6 peaks/min in AGGs and NONs during RI (NONs intruder absent vs. present, n=6, t(5)=3.070, p=0.023; AGGs intruder absent vs. present, n=5, t(4)=5.063, p=0.0072; AGGs vs. NONs (AGGs, n=5, NONs, n=6, t(9)=3.943, p=0.0034). FIG. 1C, NON representative LHb GCaMP traces during baseline (left) and RI (right). FIG. 1D, AGG representative LHb GCaMP traces during baseline (left) and RI (right). FIG. 1E, AGG LHb GCaMP activity before/after a bite (n=8 animals, average of 5 bites/animal, t(7)=5.780, p=0.0007). FIG. 1F, NON LHb GCaMP activity before/after approach (n=5 NONs, 5 approaches/animal, t(4)=3.45, p=0.026). FIG. 1G, AGG LHb GCaMP activity before/after withdrawal (n=5, avg. of 5 withdrawals/animal, t(4)=3.991, p=0.0174). FIG. 1H, NON LHb GCaMP activity before/after withdrawal (n=5, average of 5 withdrawals/animal, t(4)=3.043, p=0.0383. FIG. 1, AGG LHb GCaMP6 activity during aggression CPP (n=4, t(3)=2.352, p=0.10). FIG. 1J, NON LHb GCaMP6 activity during aggression CPP (n=4, t(3)=2.352, p=0.0458). Data are expressed as mean+SEM. * p<0.05, **p<0.01, ***p<0.001.

FIGS. 2A-2E. Orexin signaling in the LHb (lateral habenula) regulates aggression and the valence of social encounters. FIG. 2A, Experimental scheme and viral expression, scale bar=150 μm. FIG. 2B, Attack latency (left), total attack duration (middle), and aggression CPP score (right) in Scr-ctrl and shOxR2 mice (n=10-11 scr-ctrl, n=12 shOxR2; latency, t(21)=1.797, p=0.0868; duration, t(21)=2.122, p=0.459; CPP, t(20)=2.211, p=0.038). FIG. 2C, Experimental scheme and viral expression, scale bar (top)=75 μm, scale bar (bottom)=125 μm. FIG. 2D, Attack latency (left), total attack duration (middle), and aggression CPP score in YFP and ChR2 mice (n=9-10 YFP, n=10 ChR2; latency, t(17)=2.2176, p=0.044; duration, t(17)=2.496, p=0.0232; CPP, t(18)=1.642, p=0.118). FIG. 2E, Attack latency (left), total attack duration (middle), and aggression CPP score (right) in YFP and NpHR mice (n=9-11 YFP, n=8-10 NpHR; latency, t(15)=3.462, p=0.0035; duration, t(15)2.380, p=0.031; CPP, t(19)=2.933, p=0.008). All data are expressed as mean+SEM. *p<0.05, **p<0.01.

FIGS. 3A-3E. Functional and anatomical characterization of the LH-LHb orexin circuit (the lateral hypothalamus-lateral habenula orexin circuit). FIG. 3A, Representative in-situ hybridization images showing OxR2 expression in GAD2-positive and vGlut2-positive LHb (lateral habenula) neurons, scale bar=40 μm. FIG. 3B, Experimental scheme for orexin-A bath application experiments. FIG. 3C, GFP-positive cell responses to orexin-A (n=9 cells, 6 mice; Kruskall-Wallis statistic=9.839, p=0.0073, effect of treatment; Dunn's posthoc, baseline vs. orexin-A, p<0.01). FIG. 3D, Experimental scheme for GAD2 optogenetic stimulation experiments. FIG. 3E, GFP-negative (putative vGlut2-positive) responses to GAD2 optical stimulation (left) and representative trace of an optically-elicited inhibitory post-synaptic current (right).

FIGS. 4A-4D. LHb (lateral habenula) GAD2 neurons regulate aggression and the valence of aggressive social encounters. FIG. 4A, Experimental scheme and viral expression, scale bar=125 μm. FIG. 4B, Change in attack latency (left), change in total attack duration (middle), and aggression CPP score (right) in YFP and ChR2 mice (n=8-9 per group; latency, t(17)=2.176, p=0.044; duration, t(17)=2.543, p=0.0234; CPP, t(14)=2.482, p=0.0264). FIG. 4C, Experimental scheme and viral expression, scale bar=200 μm. FIG. 4D, Attack latency (left), total attack duration (middle), and aggression CPP (right) in miR-scrambled and miR-OxR2 mice (n=12 per group; latency, t(22)=2.322, p=0.029; duration, t(22)=2.950, p=0.0076; CPP, t(22)=2.155, p=0.0424). All data are expressed as mean+SEM. *p<0.05, ***p<0.001.

FIGS. 5A-5G. Lateral habenula (LHb) fiber photometry in Resident Intruder (RI) and aggression conditioned-place preference (CPP). FIG. 5A, 3-day attack latencies for aggressors (AGGs) and non-aggressors (NONs) used in photometry experiments (n=6 AGGs, one-way ANOVA, F(2)=7.248, p=0.011; Bonferroni posthoc day 1 compared to day 3, t(5)=3.620, p<0.05). FIG. 5B, AGG and NON aggression CPP scores (n=4 per group, t(6)=5.591, p=0.0014). FIG. 5C, AGGs displayed significantly lower LHb activity in than NONs in RI overall and on day 3 (n=6 NONs, n=5 AGGs; 2-way ANOVA, significant effect of phenotype (F(1)=12.97, p=0.013) and phenotype x day interaction (F(2)=4.895, p=0.0154); day 3, Bonferroni posthoc day 3 NONs compared to AGGs, t(9)=4.599, p<0.0001). FIG. 5D, AGG and NON LHb GCaMP6 responses to novel objects across days (n=6 NONs, n=5 AGGs, 2-way ANOVA, F(2)=0.4739 and p=0.4739 (interaction), F(1)=0.2896 and p=0.5949 (phenotype), F(2)=1.403 and 0.2632 (day)). FIG. 5E, AGG LHb GCaMP signal before and after random time points during RI (n=5 AGGs, 5 time points/AGG, t(4)=0.533, p=0.6221). FIG. 5F, NON LHb GCaMP signal before and after random time points during RI (n=5 NON, 5 time points/NON, t(4)=0.6545, p=0.5845). FIG. 5G, Negative correlation of LHb GCaMP activity in the paired context (expressed as % unpaired peaks) with aggression CPP score in AGGs and NONs (n=8, Pearson r=−0.8274, R2=0.7013, p=0.0095). **p<0.01, ***p<0.001. All data are expressed as mean+SEM.

FIGS. 6A-6H. LHb OxR2 expression following social encounters in the RI test. FIG. 6A, Experimental scheme. FIG. 6B, Attack latency data for AGGs and NONs for qPCR experiments. FIG. 6C, Quantification of orexin receptor (OxR) mRNA expression in the LHb of AGGs and NONs following repeated exposure to the RI test (4 h: n=7 NONs, n=8 AGGs, t(13)=2.496, p=0.0268; 24 h: n=8 per group, t(14)=2.415, p=0.03). FIG. 6D, qPCR for OxR1 and OxR2 in paraventricular thalamus (PVT), nucleus accumbens (NAc), LHb, and amygdala (Amg), and (n=7-10 per group). FIG. 6E, Representative images of orexin-A immunostaining (IHC) in the LHb, scale bar=200 μm. FIG. 6F, Representative images of in-situ hybridization (ISH) labeling of OxR2 in the LHb, scale bar=50 μm. FIG. 6G, Experimental scheme for G-deleted Rabies tracing experiment. FIG. 6H, Representative images of LHb G-deleted Rabies eGFP infection, scale bar 150 μm (left); LH eGFP-positive cell bodies from the same animal co-stained with orexin-a (red) and GFP (green), scale bar 150 μm (middle); Zoomed-in images of LH eGFP-positive cell bodies co-stained with orexin-a and GFP, scale bar=75 μm (right). All data are expressed as mean+SEM. *p<0.05.

FIGS. 7A-7C. ShOxR2 and orexin-cre validation. FIG. 7A, In-vivo qPCR of OxR2 in the LHb of mice injected with shOxR2 or scr-ctrl viruses (n=7 scr-ctrl, n=8 shOxR2, t(13)=2.458, p=0.0288). FIG. 7B, Locomotor activity in the open field of mice treated with scr-ctrl and shOxR2 viruses (n=9 scr-ctrl, n=10 shOxR2, t(18)=0.4483, p=0.6596). FIG. 7C, Validation of orexin neuron-specific infection with AAV-DIO-ChR2 in orexin-Cre mice (n=3 mice, average of 2 slices/animal, t(2)=9.443, p=0.0111). All data are expressed as mean+SEM. *p<0.05, **p<0.01.

FIGS. 8A-8H. Systemic antagonism of OxR2 reduces aggression valence. FIG. 8A, Experimental scheme for OxR2 systemic antagonism RI experiment. FIG. 8B, Experimental scheme for OxR2 systemic antagonism aggression CPP and locomotion experiments. FIG. 8C, RI test attack latency in animals treated with EMPA and vehicle (n=11 per group, t(10)=0.3215, p=0.758). FIG. 8D, RI test attack duration in animals treated with EMPA and vehicle (n=11 per group, t(10)=2.888, p=0.016). FIG. 8E, Locomotor activity in the open field for animals treated with EMPA and vehicle (n=11 per group, t(10)=0.1301, p=0.8991). FIG. 8F, Aggression CPP for animals treated with EMPA and vehicle (n=11 per group, t(22)=2.885, p=0.0086). FIG. 8G, Representative fiber photometry traces in an animal treated with vehicle and EMPA. FIG. 8H, LHb GCaMP peaks during RI during vehicle and EMPA treatment (n=5, t(4)=2.946, p=0.0421). *denotes p<0.05, **denotes p<0.01. All data are expressed as mean+SEM. *p<0.05, **p<0.01.

FIGS. 9A-9D. Further functional and anatomical characterization of the LH-LHb orexin circuit. FIG. 9A, Representative in-situ hybridization images of neurons containing GAD2 mRNA in the LHb, scale bar=200 μm. FIG. 9B, Pie chart of LHb neural types as determined by in-situ hybridization for GAD2 and vGlut2. FIG. 9C, OxR2 mRNA expression per LHb GAD2 and vGlut2 neuron (n=6 animals (1-2 slices per animal) per group, t(10)=15.67, p<0.0001). FIG. 9D, Percent OxR2-expressing GAD2 and vGlut2 LHb neurons (n=3 animals (1-2 slices per animal), t(4)=18.02, p<0.0001). All data are expressed as mean+SEM. ***p<0.001.

FIGS. 10A-10C. Fos mRNA expression following optogenetic stimulation of orexin terminals in the LHb. FIG. 10A, Schematic and experimental timeline for surgeries, optogenetic manipulations, and in-situ hybridization. FIG. 10B, Optogenetic stimulation of orexin terminals in the LHb increased Fos colocalization with GAD2 and decreased Fos colocalization with vGlut2 and DAPI (n=5 per group; GAD2: t(8)=3.854, p=0.0048; vGlut2: t(8)=3.236, p=0.0120; DAPI: t(8)=2.340, p=0.0475). FIG. 10C, Representative images of Fos expression in LHb GAD2 cells (left) and vGlut2 cells (right) from YFP and ChR2 mice (left). Scale bar=40 μm. *denotes p<0.05, ** denotes p<0.01. All data are expressed as mean+SEM.

FIGS. 11A-11E. LHb cell type-specific changes in Fos mRNA following aggression. FIG. 11A, Experimental scheme. FIG. 11B, Attack latency data for animals used in Fos RI experiments. FIG. 11C, Quantification of Fos mRNA in LHb total, GAD2, and vGlut2 neurons in AGGs and NONs following RI (n=5 AGGs, n=6 NONs, total: t(9)=2.828, p=0.0198; GAD2: t(9)=2.686, p=0.025; vGlut2: t(9)=3.421, p=0.0076). FIG. 11D, Representative images of in-situ hybridization for LHb Fos (red) and GAD2 (green) mRNA in AGGs and NONs following RI, scale bar=40 μm. FIG. 11E, Representative images of in-situ hybridization for LHb Fos (red) and vGlut2 (blue) mRNA in AGGs and NONs following RI, scale bar=40. All data are expressed as mean+SEM. *p<0.05.

FIGS. 12A-12E. LHb cell type-specific changes in OxR2 mRNA following aggression. FIG. 12A, Experimental scheme. FIG. 12B, Attack latency data for mice used for Fos in-situ hybridization experiments. FIG. 12C, OxR2 mRNA expression in AGG and NON GAD2 and vGlut2 LHb neurons following RI (n=5 NONs, n=6 AGGs, GAD2 neurons: t(9)6.039, p=0.0002; vGlut2 neurons: t(9)=0.6735, p=0.5175). FIG. 12D, Representative images of in-situ hybridization for LHb OxR2 (green) and GAD2 in AGGs and NONs (red), scale bar=30 μm. FIG. 12E, Representative images for LHb OxR2 (green) and vGLUT2 (blue) (FIG. 12D) in AGGs and NONs, scale bar=30 μm. All data are expressed as mean+SEM. ***p<0.001.

FIGS. 13A-13F. Validation of miR-OxR2 construct. FIG. 13A, In-vitro qPCR validation of miR-OxR2 construct in neuro-2A cells (n=3 per group; OxR2(left): One-way ANOVA, F(3)=5.849, p=0.039, Bonferroni posthoc miR-OxR2 vs. miR-VPK control (p<0.05), miR-OxR2 vs control (p<0.05); OxR1 (right): One-way ANOVA, F(3)=0.0738, p=0.9297). FIG. 13B, Representative in-situ hybridization images of LHb GAD2 neurons (blue) infected with the DIO-miR-OxR2 virus (green) and stained for OxR2 (red), scale bar=10 μm. FIG. 13C, Quantification of in-vivo knockdown of OxR2 mRNA with DIO-miR-OxR2 treatment (n=3 animals, 2 slices per animal, t(2)=18.84, p=0.0028). FIG. 13D, Validation of GAD2 neuron-specific infection with DIO-miR-OxR2 virus (n=3 animals, two slices per animal, t(2)=17.11, p=0.0004). FIG. 13E, Locomotor behavior in the open field in animals injected with AAV-DIO-miR-OxR2 or AAV-DIO-miR-scrambled control in the LHb (n=12 miR-scrambled, n=13 miR-OxR2, t(23)=0.4235, p=0.6759). FIG. 13F, Representative images of GAD2 (blue) co-localization with cells infected with AAV2-Ef1a-DIO-miR-OxR2-GFP (green) in the LHb. All data are expressed as mean+SEM. *p<0.05, **p<0.01.

FIGS. 14A-14D. Proportion of mice attacking for each treatment. FIG. 14A, Proportion of EMPA, seltorexant, Suvorexant, and vehicle mice attacking on day 1 of RI (Fisher's exact test, vehicle vs. EMPA p=0.0498, vehicle vs. seltorexant p=0.0498). FIG. 14B, Proportion of EMPA, seltorexant (indicated as JNJ-42847922 in the figure), Suvorexant, and vehicle mice attacking on day 3 of RI. FIG. 14C, Proportion of SR-9659 and vehicle mice attacking on day 1 of RI. FIG. 14D, Proportion of SR-9659 and vehicle mice attacking on day 3 of RI. All data represent total mice per group attacking vs. not attacking during RI. *p<0.05.

FIGS. 15A-15E. Effects of OxR antagonism on aggression. FIG. 15A, Experimental scheme. FIG. 15B, Attack latency over three days of RI for OxR2 and dual antagonists (Two-way ANOVA; main effect of drug F(3)=69.32, p<0.0001; interaction between drug and day F(6)=4.760, p=0.0003; Bonferroni posthoc EMPA vs. vehicle p<0.001 for all days, vehicle vs. seltorexant p<0.001 for all days, vehicle vs. suvorexant p<0.001 on day 3 only). FIG. 15C, Average attack duration for three days of RI for OxR2 and dual antagonists (One-way ANOVA, main effect of treatment F(3)=3.446, p=0.0281; Dunnett's multiple comparison test, vehicle vs. EMPA p<0.05, vehicle vs. seltorexant p<0.05). FIG. 15D, Attack latency over three days of RI for OxR1 antagonist (Two-way ANOVA; main effect of drug, F(1)=37.66, p<0.001; day, F(2)=6.330, p=0.0038; interaction F(2)=4.258, p=0.0204; Bonferroni posthoc vehicle vs. SR-9659 p<0.001 on days 1 and 3). FIG. 15E, Average attack latency for OxR1 antagonist (Unpaired t-test, t(15)=p=0.065). All data represent mean+SEM. *p<0.05, ***p<0.001.

FIGS. 16A-16F. Effects of orexin receptor antagonists on anxiety and locomotion. FIG. 16A, Total distance traveled in the open field for OxR2 and dual antagonists. FIG. 16B, Percent time spent in center of open field for OxR2 and dual antagonists. FIG. 16C, Total center zone entries for OxR2 and dual antagonists. FIG. 16D, Total distance traveled in the open field for OxR1 antagonist. FIG. 16E, Percent time spent in center of open field for OxR1 antagonist. FIG. 16F, Total center zone entries for OxR1 antagonist. All data represent mean+SEM.

FIG. 17. Diagram of brain showing thalamus and habenula regions, which are involved in the presently disclosed orexin system.

FIGS. 18A-18C shows three graphs representing tests performed to assess the impact of EMPA and Seltorexant on social recognition (FIG. 18A), spatial memory formation (FIG. 18B), and object recognition (FIG. 18C) in mice.

FIG. 18A, Treatment with 30 mg/kg EMPA or 20 mg/kg Seltorexant (indicated as “JNJ” on graph) did not alter the time spent with each mouse compared to vehicle treatment (one-way ANOVA). Tested compounds are shown on the x-axis with the time spent investigating is shown in seconds on the y-axis. Administration of an orexin receptor antagonist did not impair ability of an animals to distinguish between novel and familiar social targets (i.e., Juvenile mice). Untreated mice spent more time investigating the novel mouse, indicating they recognized the familiar mouse. Treatment with 30 mg/kg EMPA or 20 mg/kg seltorexant (JNJ on graph) did not alter the time spent with each mouse compared to vehicle treatment (one-way ANOVA) (FIG. 18A).

FIG. 18B, Treatment with 30 mg/kg EMPA or 20 mg/kg Seltorexant (indicated as “JNJ” on graph) did not alter the time spent with each object compared to vehicle treatment (one-way ANOVA). Tested compounds are shown on the x-axis with the time spent investigating is shown in seconds on the y-axis. Administration of an orexin receptor antagonist did not affect spatial memory formation. Untreated mice spent more time investigating the object in a new location in the second session, indicating they remember the original placement of the object. Treatment with 30 mg/kg EMPA or 20 mg/kg seltorexant (JNJ on graph) did not alter the time spent with each object compared to vehicle treatment (one-way ANOVA) (FIG. 18B).

FIG. 18C, Treatment with 30 mg/kg EMPA or 20 mg/kg Seltorexant (indicated as “JNJ” on graph) did not alter the time spent with each object compared to vehicle treatment (one-way ANOVA). Tested compounds are shown on the x-axis with the time spent investigating is shown in seconds on the y-axis. Administration of an orexin receptor antagonist did not impair recognition memory when asked to distinguish between a familiar and novel object. Untreated mice spent more time investigating the new object in the second session, indicating they remember the original object. Treatment with 30 mg/kg EMPA or 20 mg/kg seltorexant (JNJ on graph) did not alter the time spent with each object compared to vehicle treatment (one-way ANOVA) (FIG. 18C).

### DETAILED DESCRIPTION

### A. Definitions

### A, an, the

As used herein and in the claims, the singular forms “a,” “an,” and “the” include the singular and the plural reference unless the context clearly indicates otherwise. Thus, for example, a reference to “an agent” includes a single agent and a plurality of such agents.

### Aggression

As used herein, “aggression” refers to an overt hostile and harmful behavior directed to oneself and/or to others. Aggression may be verbal, non-verbal or physical. It can classified as adaptive aggression or maladaptive aggression. Verbal aggression tends to harm others mentally, while non-verbal aggression often aims to harm the social relations of others. Physical aggression on the other hand causes visible bodily injuries. In the animal kingdom, aggression is often used to influence the hierarchical structure of the particular group of animal engaged in such behavior. Additional description may be found in the references including Webster's Ninth New Collegiate Dictionary (1989), all of which are incorporated herein by reference.

An aggressive behavior can be caused by many situations. These include culture, genetics, hormones, gender, age, social setting, and emotional state. Although sometimes misused, aggression as a concept is distinct from, assertiveness, antisocial behavior, delinquency, conduct problems, disruptive behavior disorders, irritability, Oppositional Defiant Disorder, or Conduct Disorder (Dambacher et al. 2015 and De Almeida et al. 2015).

### Adaptive Aggression

As used herein, the term “adaptive aggression” refers to a main subcategory of aggression and often refers to a controlled form of aggression. It occurs as a retaliation to a provocation. The magnitude of the response tends to be equivalent to the intensity of situation that generated it. It often ceases promptly once the desired goal is achieved. This type of aggression is not indicative any form of brain structure damage, or brain function impairment (Connor, 2002; Wakefield, 1992).

### Drug-Related Aggression

As used herein, “drug-related aggression” refers to an aggressive behavior observed in an individual that results from excessive drug and/or alcohol use, or an addiction to drugs and/or alcohol, e.g., drug-related aggression associated with excessive use or addiction to opioid drugs.

### Maladaptive Aggression

As used herein, the term “maladaptive aggression” refers to a main subcategory of aggression and is a form of aggression that occurs unpredictably. It can occur without a provocation, or may happen as a result of a very minor confrontation. The magnitude of the aggression tends to be overly exaggerated or excessive when placed in the context that may have contributed to its occurrence. Maladaptive aggression does not serve identifiable goals. Therefore, it may occur as a burst, or over a prolonged period of time. It may also occur repeatedly. This type of aggression indicates a physical damage to the brain structure and/or impairment in the function of the brain. In this case, a medical intervention is advised. Maladaptive aggression can be further classified into subcategories comprising, Intermittent Explosive Disorder, Kleefstra syndrome, and Impulsive Aggression (Connor, 2002; Wakefield, 1992).

Kleefstra Syndrome

As used herein, the term “Kleefstra syndrome” refers to a genetic disorder that affects the genome at position 9q34.3. It is caused by the microdeletion of 9q34.3 and is characterized with developmental delay, intellectual disability, childhood hypotonia, speech delay and facial appearance. Some of the symptoms of Kleefstra syndrome are aggressive and emotional outbursts (Yatsenko et al. 2004).

Intermittent Explosive Disorder

As used herein, the term “intermittent explosive disorder” refers to instances of bursts and elevated levels of aggression and impulsivity. Many settings may trigger such an aggression event in individuals afflicted with this disorder. They comprise familial risk of aggression, abnormalities in neurobiological markers of aggression, trauma such as wars and explosions, and medical conditions (Coccaro et al. 1998, Coccaro 2012, Coccaro 2018 and Reardon et al. 2014).

Impulsive Aggression

As used herein, the term “impulsive aggression” refers to a subcategory of maladaptive aggression and is further defined as bursts of uncontrollable hostile behavior. Individuals suffering from this condition often have a low trigger point for aggression, and lack self-control. Impulsive aggression is also known to be associated with the serotonin pathway (Glick 2015). It was shown that mice with deleted serotonin receptor (5-HT receptor) exhibit a more violent behavior than normal mice (Nelson 2001). Impulsive aggression occurs across multiple disorders and psychiatric diseases. These comprise ADHD, autism spectrum disorder, bipolar disorder, oppositional defiant disorder, conduct disorder, intermittent explosive disorder, disruptive mood dysregulation disorder, schizophrenia, Alzheimer's disease, PTSD and disorders of traumatic stress, substance use disorder, anxiety disorders, psychosis, somatic neurological impairments such as traumatic brain injury, encephalitis, stroke and epilepsy.

### Control

As used herein, reference to a “control” or a “control group” refers to one or more subjects or participants who do not receive the experimental treatment under evaluation (e.g., orexin receptor antagonists). Typically, when conducting an experiment, these participants can be randomly selected. Typically, these participants also closely resemble the participants who are in the experimental group or the individuals who receive the treatment. For example, the control group participant may be comparable as the experimental group in terms of age, gender, disease severity, etc. While the control group does not receive treatment, it does play a critical role in the experimental process. This group serves as a benchmark, allowing researchers to compare the experimental group to the control group to see what impact a treatment had on the condition being tested. Controls may also be in the context of a cohort study. A cohort is a group of people who share a common characteristic or experience within a defined period (e.g., are currently living, are exposed to a drug or vaccine or pollutant, or undergo a certain medical procedure). Thus a group of people who were born on a day or in a particular period, say 1948, form a birth cohort. The comparison group or control group may be the general population from which the cohort is drawn, or it may be another cohort of persons thought to have had little or no exposure to the substance under investigation, but otherwise similar. Alternatively, subgroups within the cohort may be compared with each other. In other aspects, a control is in the context of a subject who is being compared during periods of on and off medication. In one example, a subject having an aggressive condition evaluated while being “on” medication, and compared to the behavior/characteristics of that subject when “off” medication.

### DORA

As used herein, reference to “DORA” or “dual OxR1/OxR2” refers to orexin receptor antagonists which bind to both OxR1 and OxR2 with the same or varying degrees of binding affinity.

### OxR1

As used herein, reference to “OxR1” means orexin-1 receptor. As used herein, an orexin receptor antagonist designated as a “OxR1” or “OxR1-selective” antagonist is selective for OxR1 and binds minimally or not at all to OxR2.

### OxR2

As used herein, reference to “OxR2” means orexin-2 receptor. As used herein, an orexin receptor antagonist designated as a “OxR2” or “OxR2-selective” antagonist is selective for OxR2 and binds minimally or not at all to OxR1.

### Subject

As used herein, a “subject” refers to any human or animal (e.g., dog, cat, livestock) exhibiting an aggressive behavior or exhibiting aggression as a symptom of one or more psychiatric disorders or a drug-related aggression. In certain embodiments, a subject can include a human exhibiting one or more neurological and psychiatric diseases or disorders that comprise ADHD, autism spectrum disorder, bipolar disorder, oppositional defiant disorder, conduct disorder, intermittent explosive disorder, disruptive mood dysregulation disorder, schizophrenia, Alzheimer's disease, PTSD and disorders of traumatic stress, substance use disorder, anxiety disorders, psychosis, somatic neurological impairments such as traumatic brain injury, encephalitis, stroke and epilepsy.

### Subject in Need

One skilled in the art will recognize that wherein methods of prevention are described (e.g., preventing an aggressive behavior in a subject taking a drug that induced aggression), a subject in need thereof (i.e., a subject in need of prevention) shall include any subject who has experienced or exhibited aggression or an aggressive behavior. Further, a subject in need thereof may additionally be a subject (preferably a mammal, more preferably a human) who has not exhibited (yet) aggression or an aggressive behavior, but who has been deemed by a physician, clinician or other medical profession to be at risk of developing aggression or an aggressive condition. For example, the subject may be deemed at risk of having new episode aggression (and therefore in need of prevention or preventive treatment) as a consequence of the subject's medical history, including, but not limited to, family history, pre-disposition, co-existing (comorbid) disorders or conditions, genetic testing, presence of an aggression-associated psychiatric disorder, and the like.

### Therapeutically Effective Amount

As used herein, “therapeutically effective amount” refers to the amount of an orexin receptor antagonist which elicits a reduction in aggression or an aggressive behavior in a subject. Preferably, the reduction in aggression or an aggressive behavior is achieved without disrupting the sleep-wake cycle, i.e., wherein the subject does not become sleepy, sedated, or otherwise fatigued as a result of the therapeutically effective amount of the orexin receptor antagonists. Preferably still, the reduction in aggression or an aggressive behavior is achieved without a detectable change in locomotion (as measured by an accepted methodology, e.g., as described in Schamhardt et al., Acta Anatomica, 1993; 146: 123-129) or gross motor skills (as measured by an accepted methodology, e.g., the Gross Motor Function Classification System—Expanded & Revised (GMFCS—E&R) as described in Dev Med Child Neurol 1997; 39:214-223). Still preferably, the reduction in aggression or an aggressive behavior is achieved without a detectable change in cognitive ability (as measured by an accepted methodology, e.g., Weintraub et al., Alzheimer's Dement, 2018; 4: 64-67). Dosages to be administered to achieve a therapeutically effective amount may be readily determined by those skilled in the art, and may vary with the mode of administration, the strength of the preparation and the advancement of the disease condition. Such factors including the particular patient being treated, including patient's sex, age, weight, diet, time of administration and concomitant diseases, among others.

### Treatment

The term “treat” or “treating” as used herein is intended to refer to administration of an active agent or composition of the invention to a subject for the purpose of effecting a therapeutic or prophylactic benefit through modulation of orexin receptor activity. Treating includes reversing, ameliorating, alleviating, inhibiting the progress of, lessening the severity of, or preventing a disease, disorder, or condition, or one or more symptoms of such disease, disorder or condition mediated through modulation of orexin receptor activity. The term “subject” refers to a mammalian patient in need of such treatment, such as a human.

### B. Orexin Signaling System

The present invention relates to the use of antagonists of the orexin signaling system as a means for treating aggression or aggressive conditions/behaviors. Additional information regarding the orexin signaling system that may be pertinent to understanding the present specification can be found, for example, in Karhu et al., BMC Struct Biol, 2015, 15:9, Bonaventure et al., J of Pharma and Experi Therapeutics, 2015, 354: 471-482, Boss et al., Expert Opinion on Therapeutic Patents, 2017, 27: 1123-1133, and Yun et al., Frontiers in Behavioral Neuroscience, 2017, 11: 1-7, each of which are incorporated herein by reference.

The orexin signaling system comprises orexin 1 and 2 receptors (OX1R and OX2R respectively) and two agonistic peptide ligands, orexin-A and orexin-B. Orexin receptors are mainly found in the central nervous system, but also in the periphery (gastrointestinal track, pancreas, adrenal gland and adipose tissue). The orexin peptides induce feeding and wakefulness. Errors in the orexin signaling system can lead to narcolepsy in humans and animals.

The orexin peptides are produced as a 131-amino acid precursor in humans, which is enzymatically cleaved to produce peptide A and B. Human orexin-A is a 33-amino acid peptide containing two intramolecular disulfide bridges (Cys6-Cys12, Cys7-Cys14), an N-terminal pyroglutamoyl residue, and an amidated C-terminus. Human orexin-B is composed of 28 residues and is amidated on its C-terminus like orexin-A, but lacks the disulfide bridges. The C-termini of orexin-A and B are highly similar (identity over 11 out of 15 amino acids), however, the N-termini are not.

The orexin receptors OX1R and OX2R are G protein-coupled receptors (GPCRs) that in human are composed of 425 amino acids (OX1R) and 444 amino acids (OX2R). The overall structure of orexin receptors consists of seven helical transmembrane segments connected by three intra- and three extracellular loops, an extracellular N-terminus and an intracellular C-terminus. The human OX1R and OX2R share a full-length pairwise sequence identity of 64%. Orexin-A is equipotent towards both receptor subtypes, whereas orexin-B is equipotent with orexin-A towards OX2R but 10-fold less potent in activating OX1R.

Orexin neuropeptides and orexin receptors play an essential and central role in regulating circadian vigilance states. In the brain, orexin neurons collect sensory input about internal and external states and send short intrahypothalamic axonal projections as well as long projections to many other brain regions. The particular distribution of orexin fibers and receptors in basal forebrain, limbic structures and brainstem regions—areas related to the regulation of waking, sleep and emotional reactivity—suggests that orexins exert essential functions as regulators of behavioral arousal; by activating wake-promoting cell firing, orexins contribute to orchestrate all brain arousal systems that regulate circadian activity, energy balance and emotional reactivity.

Small molecules have been developed to act as orexin receptor antagonists. As expected, antagonists have opposing effects to orexin peptides: reduced feeding and induction of sleep. The first drug targeting the orexin receptors, the antagonist suvorexant (Belsomra®) is marketed in the U.S. and Japan for treating insomnia.

The orexin signaling field is relatively new with the system only being first discovered in 1998. While a better understanding of the orexin signaling system has certainly been achieved since that, the role of the orexin signaling system specifically with regard to aggression or in psychiatric disorders having an aggression component was not recognized or previously understood. The present invention demonstrates for the first time that the orexin signaling system has a role in regulating aggression and further that the orexin signaling system can be therapeutically targeted with orexin receptor antagonists to clinically treat aggression. Thus, the invention meets an unmet medical need to provide effective treatments for aggression where currently there are none or very few.

### C. Orexin Receptor Antagonists

The methods of treating aggression described herein involve the administration of orexin receptor antagonists. In some embodiments, the orexin receptor antagonists have selective affinity for orexin-1 receptor (OxR1). In other embodiments, the orexin receptor antagonists have selective affinity for orexin-2 receptor (OxR2). In still other embodiments, the orexin receptor antagonists have affinity for both orexin-1 and orexin-2 receptors. Thus, in various embodiments, the methods described herein can involve the use of OxR1-selective, OxR2-selective, or dual OxR1/OxR2-selective antagonists. Many orexin receptor antagonists have been described to date and are publicly available or can be synthesized using standard chemical synthesis methods. The methods of the invention contemplate the use of any orexin receptor antagonist already known in the art, as well as any future available orexin receptor antagonists.

In one embodiment, the orexin receptor antagonist is the following compound 1:

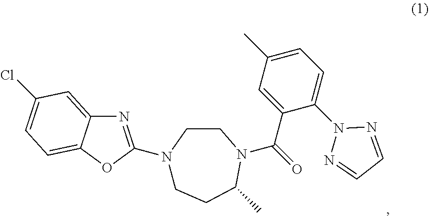 [SMILES: Cc1ccc(-n2nccn2)c(C(=O)N2CCN(c3nc4cc(Cl)ccc4o3)CC[C@H]2C)c1]

or a pharmaceutically acceptable salt thereof. This compound is otherwise known as suvorexant. The compound is a dual Ox1R/Ox2R antagonist.

In another embodiment, the orexin receptor antagonist is the following compound 2:

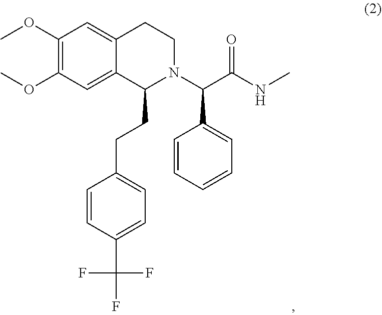

or a pharmaceutically acceptable salt thereof. This compound is otherwise known as almorexant. The compound is a dual Ox1R/Ox2R antagonist.

In still another embodiment, the orexin receptor antagonist is the following compound 3:

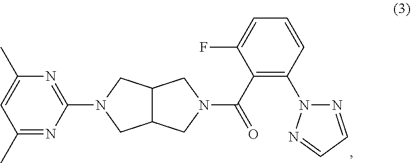 [SMILES: Cc1cc(C)nc(N2CC3CN(C(=O)c4c(F)cccc4-n4nccn4)CC3C2)n1]

or a pharmaceutically acceptable salt thereof. This compound is otherwise known as seltorexant (aka JNJ-42847992). The compound is a Ox2R-selective antagonist.

In yet another embodiment, the orexin receptor antagonist is the following compound 4:

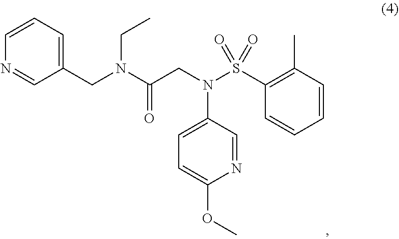 [SMILES: CCN(Cc1cccnc1)C(=O)CN(c1ccc(OC)nc1)S(=O)(=O)c1ccccc1C]

or a pharmaceutically acceptable salt thereof. This compound is otherwise known as EMPA (N-ethyl-2-[(6-methoxy-pyridin-3-yl)-(toluene-2-sulphonyl)-amino]-N-pyridin-3-ylmethyl-acetamide). The compound is a Ox2R-selective antagonist.

In still another embodiment, the orexin receptor antagonist is the following compound 5:

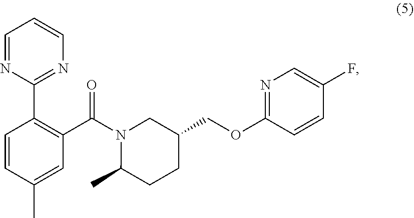 [SMILES: Cc1ccc(-c2ncccn2)c(C(=O)N2C[C@H](COc3ccc(F)cn3)CC[C@H]2C)c1]

or a pharmaceutically acceptable salt thereof. This compound is otherwise known as filorexant ([(2R,5R)-5-[(5-fluoropyridin-2-yl)oxymethyl]-2-methylpiperidin-1-yl]-(5-methyl-2-pyrimidin-2-ylphenyl)methanone). The compound is a dual Ox1R/Ox2R antagonist.

In other embodiments, the orexin receptor antagonist is the following compound 6:

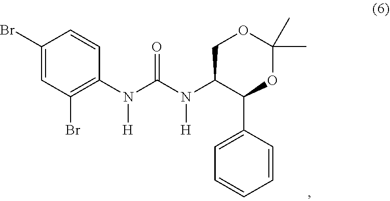 [SMILES: CC1(C)OC[C@H](NC(=O)Nc2ccc(Br)cc2Br)[C@H](c2ccccc2)O1]

or a pharmaceutically acceptable salt thereof. This compound is otherwise known as JNJ-10397049 (N-(2,4-Dibromophenyl)-N′-[(4S,5S)-2,2-dimethyl-4-phenyl-1,3-dioxan-5-yl]-urea) (see Mavanji et al (2015), “Promotion of Wakefulness and Energy Expenditure by Orexin-A in the Ventrolateral Preoptic Area,” Sleep 38, p. 1361, which is incorporated herein by reference). The compound is a Ox2R-selective antagonist.

In other embodiments, the orexin receptor antagonist is the following compound 7:

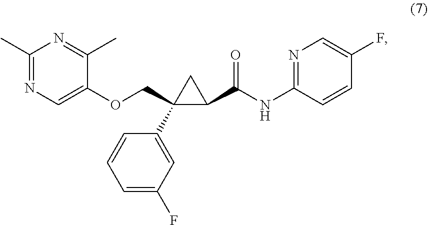 [SMILES: Cc1ncc(OC[C@@]2(c3cccc(F)c3)C[C@H]2C(=O)Nc2ccc(F)cn2)c(C)n1]

or a pharmaceutically acceptable salt thereof. This compound is otherwise known as lemborexant ((1R,2S)-2-[(2,4-dimethylpyrimidin-5-yl)oxymethyl]-2-(3-fluorophenyl)-N-(5-fluoropyridin-2-yl)cyclopropane-1-carboxamide). The compound is a dual Ox1R/Ox2R antagonist.

The orexin receptor antagonists can also include any suitable OxR1-selective, OxR2-selective, or dual OxR1/OxR2 antagonist known in the art, which can include, without limitation: N-aroyl cyclic amine derivatives (International Publication No. WO2003.002561, Jan. 9, 3003), ethylene diamine derivatives (International Publication No. WO2003051872, Jun. 26, 2003), sulfonylamino-acetic acid derivatives (International Publication No. WO2004033418, Apr. 22, 2004), N-aryl acetyl cyclic amine derivatives (International Publication No. WO200404 1791, May 21, 2004), diazepan derivatives (International Publication No. WO2007 126935, Nov. 8, 2007), amidoethylthioether derivatives (International Publication No. WO2007 126934, Nov. 8, 2007), 2-substituted proline bis-amide derivatives (International Publication No. WO2008008551, Jan. 17, 2008), bridged diazepan derivatives (International Publication No. WO2008008517, Jan. 17, 2008), substituted diazepan derivatives (International Publication No. WO2008008518, Jan. 17, 2008; US20080132490, WO2009058238), oxo bridged diazepan derivatives (International Publication No. WO2008143856, Nov. 27, 2008), 1,2-diamido ethylene derivatives (International Publication No. WO2009022311, Feb. 19, 2009), heteroaryl derivatives (International Publication No. WO20090163485, Jun. 25, 2009), methyl substituted piperidinyl derivatives (International Publication No. WO2009124956, Oct. 15, 2009), N,N-disubstituted-1,4-diazepane derivatives (Cox et al. Bioorganic & Medicinal Chemistry Letters, 2009, 19(11), 2997-3001), Orexin/Hypocretin receptor ligands (Boss, et al., Journal of Medicinal Chemistry, 2009, 52(4), 891-903) 3,9-diazabicyclo4.2.1 nonanes (Coleman et al. Bioorganic& Medicinal Chemistry Letters, 2010, 20(14), 4201-4205), the dual orexin receptor antagonist, (7R)-4-(5-Chloro-1,3-benzoxazol-2-yl)-7-methyl-1,4-diazepan-1-yl)5-methyl-2-(2H-1,2,3-triazol-2-yl)phenylmethanone, (Cox, et. al., Journal of Medicinal Chemistry, 2010 53(14) 5320-5332), pyridazine carboxamide derivatives (International Publication No. WO2010051238), 2,5-disubstituted benzamide derivatives (International Publication No WO2010051237, May 6, 2010), isonicotinamides (International Publication No WO2010051236), heterocyclylbenzoylpiperazines derivatives (International Publication No WO201048012), substituted diazepane derivatives (International Publication No WO2010.048017), substituted pyrrolidine derivatives (International Publication No WO2010.048014), triazolyl-benzoylpiperidine derivatives (International Publication No WO2010048010), triazolylbenzoylmorpholine derivatives (WO201004.8013), conformationally restrained N.N. disubstituted 1,4-diazapane derivatives (Coleman et al. Bioorganic & Medicinal Chemistry Letters, 2010, 2007), 2311-2315), tripyridyl carboxamide derivatives (International Publication No WO2010017260), imidazopyridylmethyl substituted piperidine derivatives (International Publication No WO2010.072722), imidazopyrazine substituted piperidine derivatives (US2010160344, Jun. 24, 2010; US20100160345, Jun. 24, 2010; International Publication No WO2010060472, Jun. 3, 2010), N-(1R,4S,6R)-3-(2-pyridinylcarbonyl)-3-azabicyclo[4.1.0]hept-4-yl)methyl 2-heteroarylamine derivatives (WO2010.063663), N-(1S,4S,6S)-3-(2-pyridinylcarbonyl)-3-azabicyclo[4.1.0]hept-4-yl)methyl-2-heteroarylamine derivatives (International Publication No WO2010.063662), imidazopyrimidine derivatives (International Publication No WO2010060471), the orexin-2 antagonists described in US Published Application No. 2017/0258790 A1, the octahydropyrrolo[3,4-c]pyrrol compounds described in U.S. Pat. No. 9,586,962, and imidazopyrazine derivatives (International Publication No WO2010060470).

Any orexin receptor antagonist disclosed or contemplated herein may include hydrates, solvates, or polymorphs of such compounds, and mixtures thereof, even if such forms are not listed explicitly. Solvates include those formed from the interaction or complexation of orexin receptor antagonist compounds of the invention with one or more solvents, either in solution or as a solid or crystalline form. In some embodiments, the solvent is water and then the solvates are hydrates. In addition, certain crystalline forms of the orexin receptor antagonist compounds or their pharmaceutically acceptable salts may be obtained as co-crystals. In certain embodiments of the invention, the orexin receptor antagonist compounds disclosed herein can be obtained in a crystalline form. In other embodiments, crystalline forms of the orexin receptor antagonist compounds are cubic in nature. In other embodiments, pharmaceutically acceptable salts of orexin receptor antagonist compounds can be prepared/obtained in a crystalline form. In still other embodiments, the orexin receptor antagonist compounds utilized herein can be obtained in one of several polymorphic forms, as a mixture of crystalline forms, as a polymorphic form, or as an amorphous form.

Certain orexin receptor antagonist compounds may also be able to form a zwitterion. Terms such as zwitterion, zwitterions, and their synonyms zwitterionic compound(s) are standard IUPAC-endorsed names that are well known and part of standard sets of defined scientific names. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. Zwitterions, zwitterionic compounds, inner salts and dipolar ions in the known and well established meanings of these terms are within the scope of this invention, as would in any case be so appreciated by those of ordinary skill in the art. Because there is no need to name each and every embodiment that would be recognized by those of ordinary skill in the art, no structures of the zwitterionic compounds that are associated with the orexin receptor antagonist compounds of this invention are given explicitly herein. They are, however, part of the embodiments of this invention. No further examples in this regard are provided herein because the interactions and transformations in a given medium that lead to the various forms of a given compound are known by any one of ordinary skill in the art.

The orexin receptor antagonist compounds contemplated herein for use in the aggression treatments disclosed herein can include a pharmaceutically acceptable salt. A “pharmaceutically acceptable salt” is intended to mean a salt of a free acid or base of an orexin receptor antagonist compound that is non-toxic, biologically tolerable, or otherwise biologically suitable for administration to the subject. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. An orexin receptor antagonist compound may possess a sufficiently acidic group, a sufficiently basic group, or both types of functional groups, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

In the case of nitrogen-containing orexin receptor antagonist compounds, the desired pharmaceutically acceptable salts may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid, glutaric acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

In the case of carboxylic acid-containing or sulfonic acid-containing orexin receptor antagonist compounds, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as N-methyl-D-glucamine, lysine, choline, glycine and arginine, ammonia, carbonates, bicarbonates, primary, secondary, and tertiary amines, and cyclic amines, such as tromethamine, benzylamines, pyrrolidines, piperidine, morpholine, and piperazine, and inorganic salts derived from sodium, calcium, potassium, magnesium, manganese, iron, copper, zinc, aluminum, and lithium.

The orexin receptor antagonist compounds contemplated herein may also include pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to an active orexin receptor antagonist). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Exemplary prodrugs include compounds having an amino acid residue, or a polypeptide chain of two or more (e.g., two, three or four) amino acid residues, covalently joined through an amide or ester bond to a free amino, hydroxy, or carboxylic acid group of an orexin receptor antagonist compound. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

### D. Orexin Receptor Antagonist Assays/Mouse Models

The utility of the orexin receptor antagonist compounds in accordance with the present invention may be readily determined without undue experimentation by methodologies well known in the art.

For example in a first approach, the “FLIPR Ca²⁺ Flux Assay” (Okumura et al., Biochem. Biophys. Res. Comm. 280:976-981, 2001) may be used to assay the activity of any given orexin receptor antagonist. In a typical experiment the OX1R and OX2R antagonistic activity of the compounds of the present invention could be determined in accordance with the following experimental method. For intracellular calcium measurements, Chinese hamster ovary (CHO) cells expressing the rat orexin-1 receptor or the human orexin-2 receptor, are grown in Iscove's modified DMEM containing 2 mM L-glutamine, 0.5 g/ml G418, 1% hypoxanthine-thymidine supplement, 100 U/ml penicillin, 100 μg/ml streptomycin and 10% heat-inactivated fetal calf serum (FCS). The cells are seeded at 20,000 cells/well into Becton-Dickinson black 384-well clear bottom sterile plates coated with poly-D-lysine. All reagents were from GIBCO-Invitrogen Corp. The seeded plates are incubated overnight at 37° C. and 5% CO₂. Ala-6,12 human orexin-A as the agonist is prepared as a 1 mM stock solution in 1% bovine serum albumin (BSA) and diluted in assay buffer (HBSS containing 20 mM HEPES, 0.1% BSA and 2.5 mM probenecid, pH7.4) for use in the assay at a final concentration of 70 pM. Test compounds are prepared as 10 mM stock solution in DMSO, then diluted in 384-well plates, first in DMSO, then assay buffer. On the day of the assay, cells are washed 3 times with 100 μl assay buffer and then incubated for 60 min (37° C., 5% CO₂) in 60 μl assay buffer containing 1 μM Fluo-4AM ester, 0.02% pluronic acid, and 1% BSA. The dye loading solution is then aspirated and cells are washed 3 times with 100 μl assay buffer. 30 μl of that same buffer is left in each well. Within the Fluorescent Imaging Plate Reader (FLIPR, Molecular Devices), test compounds are added to the plate in a volume of 25 μl, incubated for 5 min and finally 25 μl of agonist is added. Fluorescence is measured for each well at 1 second intervals for 5 minutes and the height of each fluorescence peak is compared to the height of the fluorescence peak induced by 70 pM Ala-6,12 orexin-A with buffer in place of antagonist. For each antagonist, IC⁵⁰ value (the concentration of compound needed to inhibit 50% of the agonist response) is determined.

In another approach, antagonist compound potency can be assessed by a radioligand binding assay (described in Bergman et. al. Bioorg. Med. Chem. Lett. 2008, 18, 1425-1430) in which the inhibition constant (Ki) is determined in membranes prepared from CHO cells expressing either the OX1 or OX2 receptor. The intrinsic orexin receptor antagonist activity of a compound which may be used in the present invention may be determined by these assays.

The orexin receptor antagonists that are useful in the instant invention can have an IC⁵⁰ of about 0.1 nM to 2000 nM, or about 1 nM to 1000 nM, or about 10 nM to about 500 nM, or about 20 nM to about 250 nM, or about 30 nM to about 150 nM, or about 40 nM to about 100 nM, or between 50 nM to 75 nM using either the first or second approach outlined above for measuring antagonist potency. The antagonist potency of the compounds in the scope of the invention can range from 0.1 nM to 100 nM, or from 10 nM to 200 nM, or from 50 nM to 300 nM, or from 100 nM to 400 nM, or from 150 nM to 500 nM, or from 200 nM to 600 nM, or from 250 nM to 700 nM, or from 300 nM to 800 nM, from 350 nM to 900 nM, from 400 nM to 1000 nM, from 500 nM to 2000 nM.

### Locomotion Assays

In aspects involving testing laboratory animals, the effects of the orexin receptor antagonists disclosed on locomotion may be evaluated by an accepted methodology. For example, the rotarod test can be conducted. The rotarod apparatus (IITC Life Sciences, Woodland Hills, Calif., USA) consists of a rod that is suspended from the floor of an apparatus. The rotational speed of the rod can be varied as desired. Test animals can first be trained for a period of time, e.g., over 2 days (e.g., 2-3 trials) to walk on the rod rotating at a constant speed (e.g., 12 rpm). Test animals can then be selected for inclusion in compound assays if they are able to remain on the rod for 120 seconds as it was accelerated, e.g., from 4 to 20 rpm (over 150 seconds). Animals unable to perform the training task were excluded from further testing. The animals can then be administered a test compound (e.g., orexin receptor antagonist) and then returned to their home cage for a period of time. The animals are then placed on the rotarod which is rotationally accelerated. Latency to fall (time to the animal falling from the apparatus) for each animal is digitally recorded by trip plates in the platform which are triggered as soon as the animal falls from the rod. One testing trial can be performed for each animal. See Ramirez et al., Front Neurosci., 2013, 7: 254.

### Aggression Assays

In other aspects, testing laboratory animals for the effects of the orexin receptor antagonists on aggression may be evaluated using an accepted methodology. For example, the effects of a compound on aggression in a test animal may be tested using the “resident intruder (RI) test,” which is an well-accepted methodology and is further described in Koolhaas et al., “The Resident-intruder Paradigm: A Standard Test for Aggression, Violence and Social Stress,” J Vis Exp., 2013; 77: 4367, which is incorporated herein by reference. The resident-intruder paradigm can be used to study offensive aggression, defensive behavior, violence and social stress in rats and, with some small modifications for other rodent species as well. When studying aggression, principally all rat strains can be used. However, strains are not equally suitable. Depending on the exact purpose of the experiment, some specific characteristics of the animals should be considered. Taken together, the resident intruder paradigm allows research on both the causes and the consequences of aggressive behavior. It is a model with a high face and construct validity that covers not only the adaptive biology of social behavior, but can be used to study maladaptive aspects as well in terms of violence and social stress pathology.

Small animal models are generally widely used for studying aggression and translating the findings to the corresponding human conditions. Such experiments will be well-known to those of ordinary skill in the art. Reference is particularly made to Haller et al., “Normal and abnormal aggression: human disorders and novel laboratory models,” Neuroscience and Biobehavioral Reviews, 2006, 30: 292-303, and Miczek et al., “Excessive aggression as model of violence: a critical evaluation of current preclinical methods,” Psychopharmacology, 2013, 226: 445-458, each of which are incorporated herein by reference.

### E. Aggression and Psychiatric Disorders

The orexin receptor antagonists disclosed herein may be used to treat aggression or aggressive behavior, in general. The orexin receptor antagonists may also be used to treat psychiatric disorders that show aggression as a symptom. The method of the current invention comprises administering a therapeutically effective amount of a orexin receptor antagonist to a subject afflicted with aggression related to a clinical condition.

In some cases the orexin receptor antagonist is used to treat impulsive aggression. In some cases the orexin receptor antagonist is used to treat intermittent explosive disorder. In some other cases the orexin receptor antagonist is used to treat X-Linked Intellectual Disability-Hypotonia-Facial Dysmorphism-Aggressive Behavior. X-Linked Intellectual Disability-Hypotonia-Facial Dysmorphism-Aggressive Behavior is a condition characterized by severe intellectual deficit, hypotonia, mild facial dysmorphism, and aggressive behaviour. This disorder has been described in 10 male members spanning four generations of one family. The facial dysmorphism includes a high forehead, prominent ears, and a small pointed chin. Height and head circumference are reduced. This disorder is transmitted as an X-linked recessive trait and the causative gene maps to Xp22. See Berry-Kravis et al., Sci Transl. Med., 2016, 8:321.

In some embodiments, the orexin receptor antagonists can be used to treat drug-induced aggression. For example, an orexin receptor antagonist may be used to treat aggression due to recreational drugs such as marijuana, ketamine, oxymorphone, N,N-Dimethyltryptamine, carisoprodol, methadone, lorazepam, morphine, buprenorphine, heroin, zolpidem, valium, clonazepam, methylphenidate, methamphetamine, tramadol, lysergic acid diethylamide, 3,4-Methylenedioxymethamphetamine, alprazolam, oxycodone, cocaine, amphetamine, hydrocodone, tobacco, and alcohol.

In other embodiments, the orexin receptor antagonists can be used to treat prescription drug-induced aggression, including anabolic steroids and benzodiazepines. In some cases, the orexin receptor antagonists can be used to treat aggression associated with social isolation.

In some cases, the orexin receptor antagonist can be used to treat aggression associated with multiple mental health disorders, for example, Kleefstra syndrome, Gaucher disease type III, Hunters syndrome (MPS III), Alzheimer's disease, Parkinson's disease, Huntington's disease, ADHD, Dementia (frontotemporal, lewy body), Schizophrenia, Bipolar disorder, Autism spectrum disorder, Conduct disorder, Oppositional defiant disorder and Personality disorders.

As previously stated, Kleefstra syndrome herein refers to a genetic disorder that affects the genome at position 9q34.3. It caused by the microdeletion of 9q34.3, and characterized developmental delay, intellectual disability, childhood hypotonia, speech delay and facial appearance. Some of the symptoms of Kleefstra syndrome are aggressive and emotional outbursts (Yatsenko et al. 2004).

Gaucher disease type III also known as chronic neuronopathic Gaucher disease, is late onset neuronopathy. It is characterized by a gradual onset, and comprises symptoms including, seizures, skeletal irregularities, eye movement disorders, cognitive problems, poor coordination, enlarged liver and spleen, respiratory problems and blood disorders. People with the disease may survive into adulthood.

Hunters syndrome (MPS III) is a lysosomal storage disorder caused by lysosomal enzyme deficiency. The lack of enzyme causes heparan sulfate and dermatan sulfate to accumulate in all body tissues. MPS III is clinically characterized also by progressive neurodegeneration (de Ruijter et al. 2013).

Alzheimer's disease is a chronic neurodegenerative disease that usually starts slowly and worsens over time. It is characterized by the formation of extracellular deposits of amyloid beta plaques in the brain. It is the cause of 60-70% of cases of dementia. The most common early symptom is difficulty in remembering recent events (short-term memory loss), followed by problems with language, disorientation, mood swings, loss of motivation, and behavioral issues. This disease gradually leads to death. Although the speed of progression can vary, the typical life expectancy following diagnosis is three to nine years (Henry et al. 2010 and Todd et al. 2013).

Parkinson's disease is a neurodegenerative disease characterized by cell death in the brain's basal ganglia, affecting up to 70% of the dopamine secreting neurons in the substantia nigra. This in turn causes impaired motor functions such as tremors. It is a slow progressing disease. The most common symptoms of Parkinson's disease are depression and anxiety (Braak and Braak 2000, Sveinbjornsdottir 2016).

Huntington's disease (HD) is an inherited autosomal dominant inherited neurodegenerative disease characterized by progressive motor, behavioral, and cognitive decline, ultimately culminating in death (Dayalu, and Albin 2015).

ADHD is often classified as a mental disorder of the neurodevelopmental type, since it is mostly diagnosed in children. It's symptoms include difficulty focusing and hyperactivity.

The compounds of the invention may also be used to treat aggression associated with children having conduct disorder (CD). Children with CD behave aggressively toward others or violate rules or laws—for example, by skipping school or shoplifting. There are four categories of conduct disorder behaviors: aggressive, destructive, deceitful, and violating rules. Aggressive behavior includes threatening or harming people or animals, fighting with others, using weapons, and committing sexual assault. Destructive behavior involves damaging property. Deceitful behavior may include repeated lying, shoplifting, or stealing. A child who violates rules may engage in inappropriate behavior, such as running away from home, skipping school, or being sexually active at a very young age.

When treating the aggressive symptoms of a psychiatric disorder, such as, Kleefstra syndrome, Gaucher disease type III, Hunters syndrome (MPS III), Alzheimer's disease, Parkinson's disease, Huntington's disease, ADHD, Dementia (frontotemporal, lewy body), Schizophrenia, Bipolar disorder, Autism spectrum disorder, Conduct disorder, Oppositional defiant disorder and Personality disorders, the orexin receptor antagonists can be administered in a therapeutically effective amount in combination with (i.e., meaning at the same time, in the same formulation, at the same time with different or separate formulations, or at different times and in any order or sequence) one or more therapies (e.g., standard of care treatment) for treating the psychiatric disorder.

### F. Methods of Treating Aggression

The disclosure provides for methods of treating aggression or aggressive behavior in a subject by administering a therapeutically effective amount of one or more orexin receptor antagonists, or a pharmaceutical composition comprising same. A subject administered with a compound of the present invention, or a pharmaceutically acceptable salt thereof, is generally a mammal, such as a human being, male or female. The amount of compound administered to the subject is an amount sufficient to antagonize the orexin receptor (e.g., OxR1-selectively, OxR2-selectively, or dual OxR1/OxR2, depending on the antagonist used) in the subject. It is recognized that one skilled in the art may treat existing symptoms of aggression or by prophylactically treating a subject likely to express aggression, with an effective amount of a compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a subject that is predisposed to such disease or disorder. The terms “administration of” and or “administering a” compound should be understood to mean providing a compound of the invention or a prodrug of a compound of the invention to the subject.

As described herein, the inventors found a surprising, robust aggression-reducing effect when using the compounds described on subjects diagnosed with aggression or having a psychiatric disorder that has aggression as a symptom. Preferably, the administration can occur without inducing negative effects on the sleep/wake cycle associated with the orexin system, and without negative effects on cognition and/or locomotion or gross motor function.

The dose is the amount of drug taken at any one time. This can be expressed in a variety of ways known in the art, e.g., as the weight of drug (e.g., 250 mg), volume of drug solution (e.g., 10 mL, 2 drops), the number of dosage forms (e.g., 1 capsule, 1 suppository) or some other quantity (e.g., 2 puffs). The dosage regimen is the frequency at which the drug doses are administered. Non-limiting examples include 2.5 mL twice a day, one tablet three times a day, and one injection every four weeks. The total daily dose is calculated from the dose and the number of times per day the dose is taken. The dosage form is the physical form of a dose of drug. Common dosage forms include tablets, capsules, creams, ointments, aerosols and patches, and others listed herein. Each dosage form may also have a number of specialized forms, such as, extended-release, buccal, dispersible and chewable tablets, etc. The strength is the amount of drug in the dosage form or a unit of the dosage form (e.g., 500 mg capsule, 250 mg/5 mL suspension). The route of administration is the way the dosage form is given. Common routes of administration include oral, rectal, inhalation, nasal and topical.

As will be appreciated by those having ordinary skill in the art, e.g., a practicing clinician, physician, psychiatrist, psychologist, or other suitable medical professional in the care of a patient, there are many factors taken into consideration when deciding a dose of drug—including age of the patient, weight, sex, ethnicity, liver and kidney function and whether the patient smokes. Other medicines may also affect the drug dose. Dosage instructions can be written on a prescription or hospital chart, and on the pharmacy label of a prescribed medicine. Dosage instructions can also be found on the packaging and inserts of medicines. Effective amounts or doses of the compounds of the present invention (e.g., those doses which reduce or treat aggression without affecting another aspect relating to the orexin cycle, e.g., the sleep/wake cycle, locomotion, or cognition) may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician.

Additionally, maintenance of the anti-aggression response may be established by, for example, an absence of relapse of the aggressive symptom, an absence of the need for additional or alternate treatment(s) for the aggression, or an absence of the worsening of the aggression. The physician or attending clinician may utilize any technique known in the art including, without limitation, general patient evaluation, diagnostic questionnaires, and evaluations such as the Peer Aggressive and Reactive Behaviors Questionnaire (PARB-Q) (Borsa et al., Trends Psychiatry Psychother, 2014, vol. 36, no. 2). The frequency may be evaluated and/or changed if the score from one or more of the above-noted scales or questionnaire changes.

Also contemplated by these methods is the administration of rescue doses of the compounds described herein. The term “rescue dose” as used herein refers to one or more additional doses of a compound described herein in addition to the regularly prescribed dose. The amount of a compound described herein in the rescue dose may be determined by the prescribing physician or clinician and will depend on any of the factors discussed herein. In certain embodiments, the rescue dose of a compounds described herein is the same as the effective dose used during the normal administration schedule. In other embodiments, the rescue dose differs from the effective dose used during the normal administration schedule.

Once improvement of the patient's aggression or aggressive condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In various embodiments, the compounds can be used to treat aggressive conditions in children. To diagnose a behavioral problem in a child or adolescent, an expert trained in childhood development and behavior meets with the child to perform a comprehensive evaluation. The first part of the evaluation involves interviews-both individually and together with parents—to assess a child's background, medical history, and symptoms. When a clinician conducts the diagnostic interview with a child, he or she speaks with the child and observes the child's nonverbal communication, such as facial expressions and posture. A parent also would be asked to complete various questionnaires to give the medical professionals a sense of the child's behaviors and how they are affecting daily life. The clinicians also would talk with the child's teachers and caregivers. Once diagnosed with an aggressive condition, the child can be administered a therapeutically effective amount of the compounds of the invention.

Children and teens with oppositional defiant disorder (ODD) may also be treated with the compounds of the invention to reduce or ameliorate the aggressive symptoms and behaviors. Such patients have a recurrent pattern of defiant and hostile behavior toward parents, teachers, or other authority figures that interferes with their day-to-day lives. Behaviors include frequent tantrums, excessive arguing with adults, and refusal to comply with an adult's requests or rules. A child may try to annoy or upset people and may harbor anger or resentment. These symptoms may be more noticeable at home or at school, but they can be present in many places. Signs of the disorder usually appear before age eight, but more serious symptoms may develop later. Symptoms tend to begin gradually, then worsen over months or years. The condition is common among children and teens with ADHD. Both disorders share common symptoms of disruptive behaviors. However, children and adolescents who have both ODD and ADHD tend to be more aggressive, have more negative behavioral symptoms of ODD, and perform worse in school than those who have ODD alone. To receive a diagnosis of ODD, a child must engage in at least four of the following behaviors on a frequent basis: losing his or her temper, arguing with and defying adults, blaming others for his or her own errors or misconduct, being easily annoyed by others, or deliberately trying to annoy other people.

### Combination Therapy

The orexin receptor antagonists can be administered alone, or in combination with one or more other suitable drugs, at any effective dose, using any effective or suitable dosing regimen, and in any suitable dosing form, and administered by any suitable dosing route.

The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention is contemplated. However, the combination therapy may also include therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds.

In one embodiment, the compounds may be used in combination with additional active ingredients in the treatment of a psychiatric condition having aggression as a symptom, such as, Kleefstra syndrome, Gaucher disease type III, Hunters syndrome (MPS III), Alzheimer's disease, Parkinson's disease, Huntington's disease, ADHD, Dementia (frontotemporal, lewy body), Schizophrenia, Bipolar disorder, Autism spectrum disorder, Conduct disorder, Oppositional defiant disorder and Personality disorders. The additional active ingredients may be administered simultaneously, separately or sequentially. In some embodiments, the additional active ingredients are effective in the treatment of conditions, disorders, or diseases mediated by orexin activity, such as another orexin modulator or a compound active against another target associated with the particular condition, disorder, or disease. The combination may serve to increase efficacy (e.g., by including in the combination a compound potentiating the potency or effectiveness of a compound herein), decrease one or more side effects, or decrease the required dose of the compound described herein or additional active agent. In certain embodiments, the additional active ingredient is an antidepressant. In other embodiments, the additional active ingredient is a monoaminergic antidepressant.

In one embodiment, the orexin receptor antagonists may be used in combination with an antidepressant. The antidepressant may be a conventional drug used to combat depression such as N-methyl-D-aspartate receptor antagonists, norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), noradrenergic and specific serotonergic antidepressants (NaSSAs), corticotropin releasing factor (CRF) antagonists, alpha-adrenoreceptor antagonists and atypical antidepressants. In some embodiments, the N-methyl-D-aspartate (NMDA) receptor antagonist is ketamine including its racemates esketamine, arketamine, or combinations thereof. In further embodiments, the norepinephrine reuptake inhibitor includes amitriptyline, clomipramine, doxepin, imipramine, trimipramine, amoxapine, desipramine, maprotiline, nortriptyline, protriptyline, reboxetine, or pharmaceutically acceptable salts thereof. In other embodiments, the selective serotonin reuptake inhibitor includes fluoxetine, fluvoxamine, paroxetine, sertraline, or pharmaceutically acceptable salts thereof. In further embodiments, the monoamine oxidase inhibitor includes isocarboxazid, phenelzine, tranylcypromine, selegiline and pharmaceutically acceptable salts thereof. In yet other embodiments, the reversible inhibitor of monoamine oxidase includes moclobemide or pharmaceutically acceptable salts thereof. In still further embodiments, the serotonin and noradrenaline reuptake inhibitor includes venlafaxine or pharmaceutically acceptable salts thereof. In other embodiments, the atypical antidepressant includes bupropion, lithium, nefazodone, trazodone, viloxazine, sibutramine, or pharmaceutically acceptable salts thereof. In yet further embodiments, the second antidepressant includes adinazolam, alaproclate, amineptine, amitriptyline/chlordiazepoxide combination, atipamezole, azamianserin, bazinaprine, befuraline, bifemelane, binodaline, bipenamol, brofaromine, bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metapramine, metralindole, mianserin, milnacipran, minaprine, mirtazapine, monirelin, nebracetam, nefopam, nialamide, nomifensine, norfluoxetine, orotirelin, oxaflozane, pinazepam, pirlindone, pizotyline, ritanserin, rolipram, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine zometapine, or pharmaceutically acceptable salts thereof; or St. John's wort herb, Hypericum perforatum, or extracts thereof.

In some embodiments, the orexin receptor antagonist compounds can be co-administered with esketamine (a general anesthetic). In further embodiments, the orexin receptor antagonist compounds can be administered separately from esketamine such as, e.g., sequentially. The compounds may be administered prior or subsequent to esketamine.

The weight ratio of the compound of the present invention to the second active ingredient may be varied and will depend upon the effective dose of each ingredient. Generally, an effective dose of each will be used. In such combinations the compound of the present invention and other active agents may be administered separately or in conjunction. In addition, the administration of one element may be prior to, concurrent to, or subsequent to the administration of other agent(s).

In other embodiments, the subject compounds may be employed in combination with other therapeutic compounds which are known in the art, either administered separately or in the same pharmaceutical compositions, including, but are not limited to: insulin sensitizers including (i) PPAR.gamma. antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like); (iii) biguanides such as metformin and phenformin; (b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂); (c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide; (d) .alpha.-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like; (e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activater receptor a agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics; (f) PPARa agonists such as beclofibrate, benzafibrate, ciprofibrate, clofibrate, etofibrate, fenofibrate, and gemfibrozil; and other fibric acid derivatives, such as Atromid, Lopid and Tricor, and the like, and PPARa agonists as described in WO 97/36579; (g) PPARA agonists, such as those disclosed in WO97/28149; (h) PPAR a/A agonists, such as muraglitazar, and the compounds disclosed in U.S. Pat. No. 6,414,002; (i) anti-obesity agents, such as (1) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB 1 receptor antagonists or inverse agonists, such as rimonabant, taranabant, AMT-251, and SR-14778 and SR 141716A (Sanofi Synthelabo), SLV-319 (Solvay), BAY 65-2520 (Bayer) and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; (4) anti-obesity serotonergic agents, such as fenfluramine, dexfenfluramine, phentermine, and sibutramine; (5) 03-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A; (6) pancreatic lipase inhibitors, such as orlistat (Xenical Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027; (11) melanin-concentrating hormone 2 receptor (MCH2R) agonist/antagonists; (12) orexin receptor antagonists, such as SB-334867-A, and those disclosed in patent publications herein; (13) serotonin reuptake inhibitors such as fluoxetine, paroxetine, and sertraline; (14) melanocortin agonists, such as Melanotan II; (15) Mc4r (melanocortin 4 receptor) agonists, such as CHIR86036 (Chiron), ME-10142, and ME-10145 (Melacure), CHIR86036 (Chiron); PT-141, and PT-14 (Palatin); (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR14613, and those described in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and O-[3-(1H-imidazol-4-yl)propanol]-carbamates; (25) beta-hydroxy steroid dehydrogenase-1 inhibitors (beta-HSD-1); (26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKline), SR146131 (Sanofi Synthelabo), butabindide, PD170,292, and PD 149164 (Pfizer); (34) CNTF derivatives, such as axokine (Regeneron); (35) monoamine reuptake inhibitors, such as sibutramine; (36) UCP-1 (uncoupling protein-1), 2, or 3 activators, such as phytanic acid, 4-[(E)-2-(5,6,7,8-tetrahydro-5,5,8,8-tetramethyl-2-napthalenyl)-1-propeny-1]benzoic acid (TTNPB), retinoic acid; (37) thyroid hormone .beta. agonists, such as KB-2611 (KaroBioBMS); (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444, sitagliptin; and the compounds disclosed in U.S. Pat. No. 6,699,871, WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); (50) Topiramate (Topimax); (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)); (51) Neuropeptide Y2 (NPY2) receptor agonists such NPY3-36, N acetyl [Leu(28,31)]NPY 24-36, TASP-V, and cyclo-(28/32)-Ac-[Lys28-Glu32]-(25-36)-pNPY; (52) Neuropeptide Y4 (NPY4) agonists such as pancreatic peptide (PP), and other Y4 agonists such as 1229U91; (54) cyclooxygenase-2 inhibitors such as etoricoxib, celecoxib, valdecoxib, parecoxib, lumiracoxib, BMS347070, tiracoxib or JTE522, ABT963, CS502 and GW406381; (55) Neuropeptide Y1 (NPY1) antagonists such as BIBP3226, J-115814, BIBO 3304, LY-357897, CP-671906, GI-264879A; (56) Opioid antagonists such as nalmefene (Revex), 3-methoxynaltrexone, naloxone, naltrexone; (57) 110 HSD-1 (11-beta hydroxy steroid dehydrogenase type 1) inhibitors such as BVT 3498, BVT 2733, and those disclosed in WO 01/90091, WO 01/90090, WO 01/90092, U.S. Pat. No. 6,730,690 and US 2004-0133011; (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; and (88) zonisamide, (89) neuromedin U and analogs or derivatives thereof, (90) oxyntomodulin and analogs or derivatives thereof, and (91) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699.

In another embodiment, the subject compounds may be employed in combination with anti-Alzheimer's agents; beta-secretase inhibitors, such as verubecestat; gamma-secretase inhibitors; growth hormone secretagogues; recombinant growth hormone; HMG-CoA reductase inhibitors; NSAID's including ibuprofen; vitamin E; anti-amyloid antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil, and tacrine; growth hormone secretagogues such as ibutamoren, ibutamoren mesylate, and capromorelin; histamine H3 antagonists; AMPA agonists; PDE IV inhibitors; GABAA inverse agonists; or neuronal nicotinic agonists.

In still other embodiments, the subject compound may be employed in combination with hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amitriptyline, amobarbital, amoxapine, bentazepam, benzoctamine, brotizolam, bupropion, busprione, butabarbital, butalbital, capuride, carbocloral, chloral betaine, chloral hydrate, chlordiazepoxide, clomipramine, clonazepam, cloperidone, clorazepate, clorethate, clozapine, cyprazepam, desipramine, dexclamol, diazepam, dichloralphenazone, divalproex, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, oxazepam, paraldehyde, paroxetine, pentobarbital, perlapine, perphenazine, phenelzine, phenobarbital, prazepam, promethazine, propofol, protriptyline, quazepam, reclazepam, roletamide, secobarbital, sertraline, suproclone, temazepam, thioridazine, tracazolate, tranylcypromaine, trazodone, triazolam, trepipam, tricetamide, triclofos, trifluoperazine, trimetozine, trimipramine, uldazepam, venlafaxine, zaleplon, zolazepam, zolpidem, and salts thereof, and combinations thereof, and the like, or the subject compound may be administered in conjunction with the use of physical methods such as with light therapy or electrical stimulation.

In another embodiment, the subject compounds may be employed in combination with a nicotine agonist or a nicotine receptor partial agonist such as varenicline, opioid antagonists (e.g., naltrexone (including naltrexone depot), antabuse, and nalmefene), dopaminergic agents (e.g., apomorphine), ADD/ADHD agents (e.g., methylphenidate hydrochloride (e.g., Ritalin® and Concerta), atomoxetine (e.g., Strattera), a monoamine oxidase inhibitor (MAOI), amphetamines (e.g., Adderall®)) and anti-obesity agents, such as apo-B/MTP inhibitors, 11Beta-hydroxy steroid dehydrogenase-1 (11Beta-HSD type 1) inhibitors, peptide YY3-36 or analogs thereof, MCR-4 agonists, CCK-A agonists, monoamine reuptake inhibitors, sympathomimetic agents, beta-3 adrenergic receptor agonists, dopamine receptor agonists, melanocyte-stimulating hormone receptor analogs, 5-HT2c receptor agonists, melanin concentrating hormone receptor antagonists, leptin, leptin analogs, leptin receptor agonists, galanin receptor antagonists, lipase inhibitors, bombesin receptor agonists, neuropeptide-Y receptor antagonists (e.g., NPY Y5 receptor antagonists), thyromimetic agents, dehydroepiandrosterone or analogs thereof, glucocorticoid receptor antagonists, other orexin receptor antagonists, such as suvorexant, glucagon-like peptide-1 receptor agonists, ciliary neurotrophic factors, human agouti-related protein antagonists, ghrelin receptor antagonists, histamine 3 receptor antagonists or inverse agonists, and neuromedin U receptor agonists, and pharmaceutically acceptable salts thereof.

In another embodiment, the subject compounds may be employed in combination with an anoretic agent (appetite-reducing agent) such as aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; selective serotonin reuptake inhibitor (SSRI); halogenated amphetamine derivatives, including chlorphentermine, cloforex, clortermine, dexfenfluramine, fenfluramine, picilorex and sibutramine; and pharmaceutically acceptable salts thereof.

In another embodiment, the subject compounds may be employed in combination with an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the subject compound may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

### G. Pharmaceutical Compositions

The orexin receptor antagonist compounds described herein may be formulated as a pharmaceutical composition for administration to a subject. Accordingly, a pharmaceutical composition may comprise (a) an effective amount of at least one compound described herein and (b) a pharmaceutically acceptable excipient. A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. Such compositions are especially useful for treating aggression and aggressive behaviors.

The production of the pharmaceutical compositions can be effected in a manner which will be familiar to any person skilled in the art (see for example Mark Gibson, Editor, Pharmaceutical Preformulation and Formulation, IHS Health Group, Englewood, Colo., USA, 2001; Remington, The Science and Practice of Pharmacy, 20th Edition, Philadelphia College of Pharmacy and Science) by bringing the described compounds and their pharmaceutically acceptable salts, optionally in combination with other therapeutically valuable substances, into a administration form together with suitable, non-toxic, inert, therapeutically compatible solid or liquid carrier materials and, if desired, usual pharmaceutical adjuvants.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparations may be in the form of tablets, capsules, sachets, dragees, powders, granules, lozenges, powders for reconstitution, liquid preparations, or suppositories. Preferably, the compositions are formulated for intravenous infusion, topical administration, or oral administration. In certain other embodiments, the compositions are formulated for slow release. In certain embodiments, the compositions are formulated for immediate release.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 20% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

For oral administration, the compounds can be provided in the form of tablets or capsules, or as a solution, emulsion, or suspension. In certain embodiments, the compounds may be taken with food.

Oral tablets may include a compound mixed with pharmaceutically acceptable excipients such as inert fillers, diluents, disintegrating agents, binding agents, lubricating agents, sweetening agents, flavoring agents, coloring agents, glidants and preservative agents. Suitable inert fillers include sodium and calcium carbonate, sodium and calcium phosphate, lactose, lactose monohydrate, starch, sugar, glucose, methyl cellulose, magnesium stearate, mannitol, sorbitol, hypromellose, and the like. Exemplary liquid oral excipients include ethanol, glycerol, water, and the like. Starch, polyvinyl-pyrrolidone, sodium starch glycolate, microcrystalline cellulose, crospovidone (cross-linked polyvinyl N-pyrrolidone or PVP), and alginic acid are suitable disintegrating agents. Binding agents may include hypromellose (hydroxypropyl methylcellulose or HPMC), starch and gelatin. The lubricating agent, if present, may be magnesium stearate, stearic acid, or talc. The glidant, if present, may be silica (SiO.sub.2) such as colloidal silica. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, the compound may be mixed with a solid, semi-solid, or liquid diluent. Soft gelatin capsules may be prepared by mixing the compound with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups or may be lyophilized or presented as a dry product for reconstitution with water or other suitable vehicle before use. Such liquid compositions may optionally contain pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives. Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The compounds described herein may also be administered by non-oral routes. For example, the compounds may be formulated for rectal administration. For parenteral use, including intravenous, intramuscular, or intraperitoneal routes, the compound may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of the compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compound may utilize a patch formulation to affect transdermal delivery.

Compounds may alternatively be administered by inhalation, via the nasal or oral routes, e.g., in a spray formulation also containing a suitable carrier.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

### H. Pharmaceutical Kits

Also described herein are kits for administering one or more compounds described herein to a patient for the treatment of depression. The representative kits include one or more dosage units comprising an effective amount of one or more compounds described herein for administration to a patient and at a given frequency.

Also optionally included in the kits is an aggression symptom rating scale questionnaire. The questionnaire may be for use by the patient alone or in combination with a physician. The questionnaire may be useful for determining the level of aggression of the patient at any stage of compound administration. In one embodiment, the questionnaire is one or more of the questionnaires noted herein.

Instructions for performing the claimed methods and administering the compound may also be included in the kits described herein.

The kits may be organized to indicate a single formulation containing a compound described herein or combination of formulations, each containing a compound described herein. The composition may be sub-divided to contain appropriate quantities of a compound described herein. The unit dosage can be packaged compositions such as packeted powders, vials, ampoules, prefilled syringes, tablets, caplets, capsules, or sachets containing liquids.

The compounds described herein may be a single dose or for continuous or periodic discontinuous administration. For continuous administration, a kit may include a compound described herein in each dosage unit. When varying concentrations of a compound described herein, the components of the composition containing the compound described herein, or relative ratios of the compound described herein or other agents within a composition over time is desired, a kit may contain a sequence of dosage units.

The kit may contain packaging or a container with a compound described herein formulated for the desired delivery route. The kit may also contain dosing instructions, an insert regarding the compound described herein, instructions for monitoring circulating levels of the compound, or combinations thereof. Materials for using the compound may further be included and include, without limitation, reagents, well plates, containers, markers, or labels, and the like. Such kits may be packaged in a manner suitable for treatment of a desired indication.

Other suitable components to include in such kits will be readily apparent to one of skill in the art, taking into consideration the desired indication and the delivery route. The kits also may include, or be packaged with, instruments for assisting with the injection/administration of the compound to the patient. Such instruments include, without limitation, an inhalant, syringe, pipette, forceps, measuring spoon, eye dropper, or any such medically approved delivery means. Other instrumentation may include a device that permits reading or monitoring reactions in vitro.

The compound may be provided in dried, lyophilized, or liquid forms. When reagents or components are provided as a dried form, reconstitution generally is by the addition of a solvent. The solvent may be provided in another packaging means and may be selected by one skilled in the art.

A number of packages or kits are known to those skilled in the art for dispensing pharmaceutical agents. In certain embodiments, the package is a labeled blister package, dial dispenser package, or bottle.

### EXAMPLES

### Example 1: Orexin Inputs to GABAergic Lateral Habenula (LHb) Neurons Control the Valence of Aggression

### Abstract

Heightened aggression is characteristic of multiple neuropsychiatric disorders and can have a wide variety of negative effects on patients, their families, and the public. Recent studies in humans and animals have implicated brain reward circuits in aggression and suggest that, in subsets of aggressive individuals, repeated domination of subordinate social targets is reinforcing. Here, it is shown that orexin neurons originating from the lateral hypothalamus (“LH”) activate a small population of GABAergic interneurons in the lateral habenula (“LHb”) via orexin receptor 2 to promote aggression and conditioned preference for aggression-paired contexts. The study suggests that the orexin system is a promising target for the development of novel therapies aimed at reducing aggressive behaviors and provides the first functional evidence of a local inhibitory circuit within the lateral habenula.

### One-Sentence Summary

Activation of orexin receptor 2 on locally-inhibitory lateral habenula GABA neurons drives the positive valence of aggression.

### Introduction

Individuals suffering from several psychiatric syndromes display increased risk for pathological aggressive behaviors [1]. It is hypothesized that brain reward systems controlling the valence of social interactions may be dysregulated in these individuals, leading to heightened aggression that is positively reinforcing [2]. Recent studies in animals find that subsets of highly aggressive mice will lever press for access to subordinate intruders [3-5] and form conditioned-place preferences (CPP) for contexts that are associated with access to subordinate intruders [6]. Although hypothalamic neural circuits promoting aggression initiation have been studied extensively [7, 8], relatively little is known about reward circuit mechanisms controlling the valence of aggression [6, 9]. As the lateral hypothalamus (LH) provides a potential link between ventral hypothalamic aggression initiation circuits and habenular aggression reward circuitry [7], it was hypothesized that the LH neuropeptide orexin (hypocretin) modulates lateral habenula (LHb) activity to control aggression and its reinforcing properties.

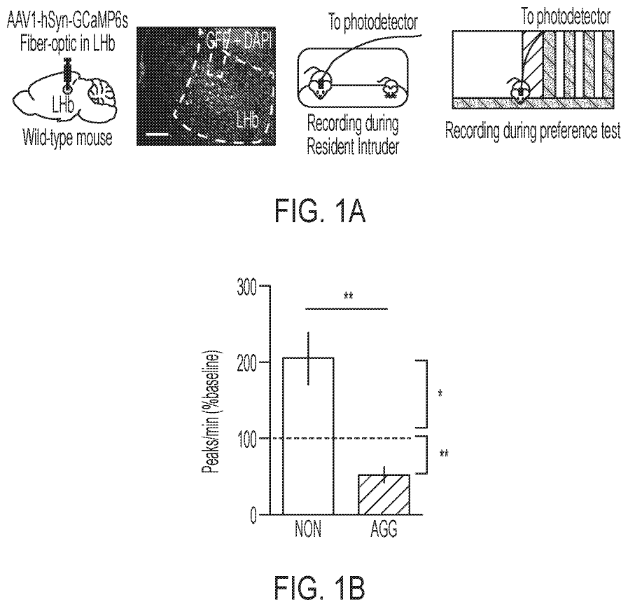

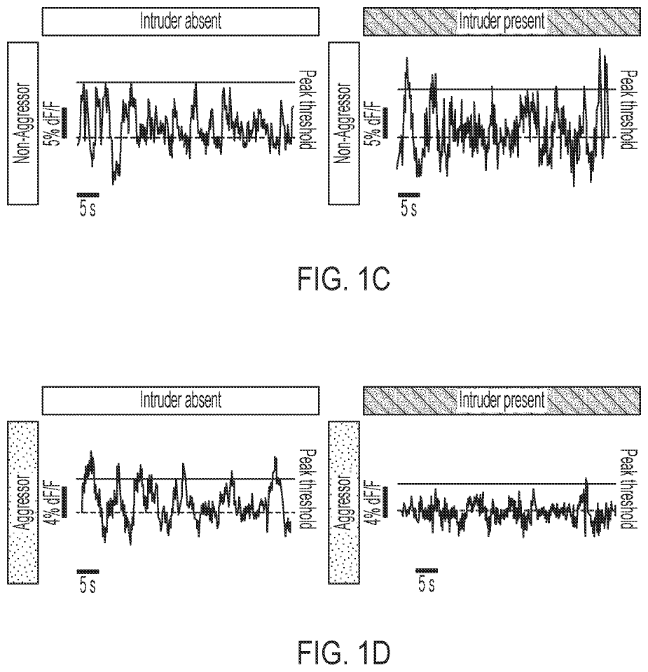

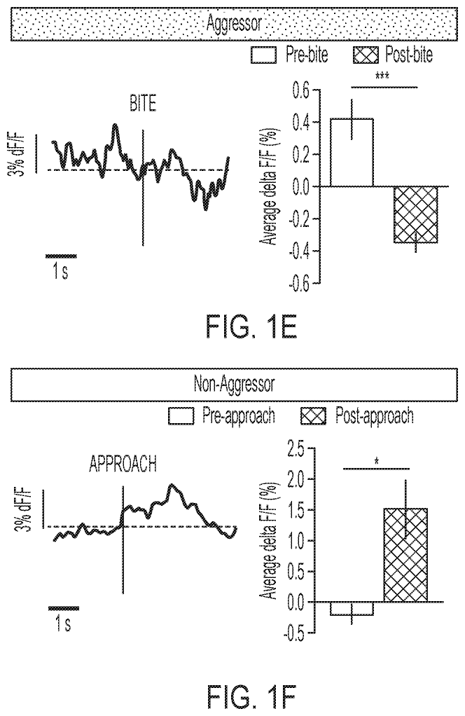

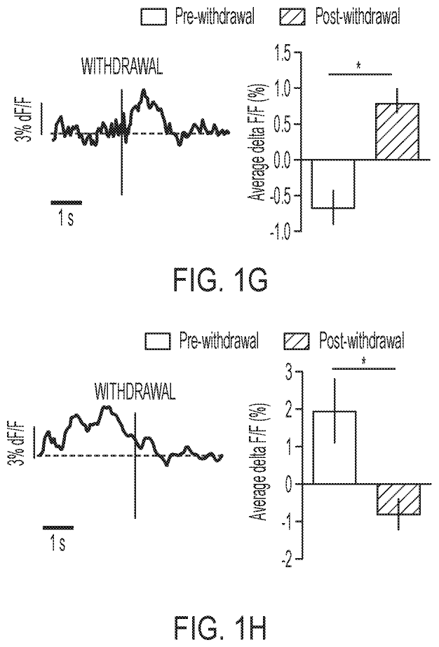

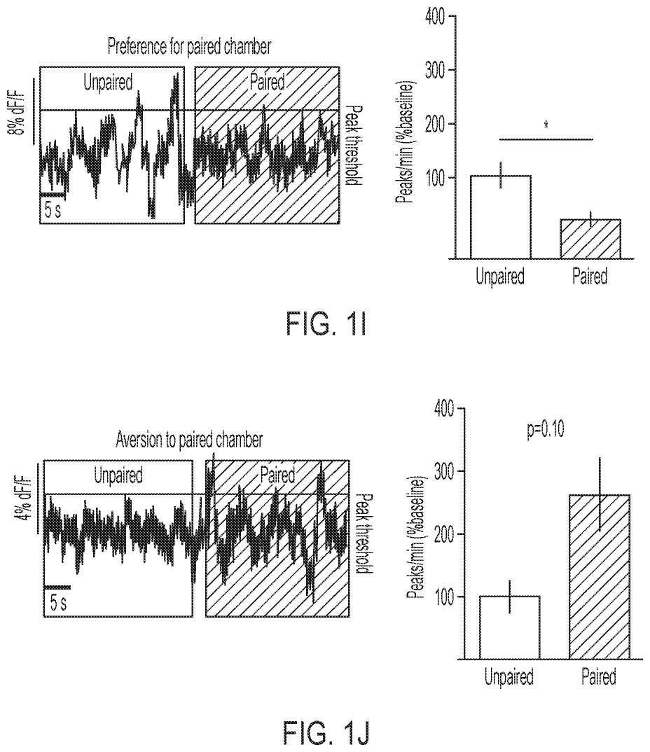

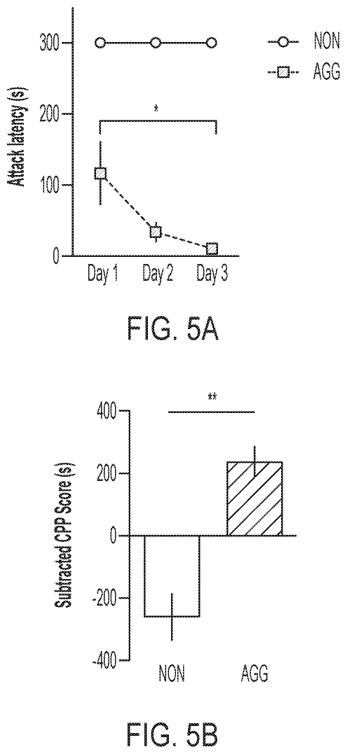

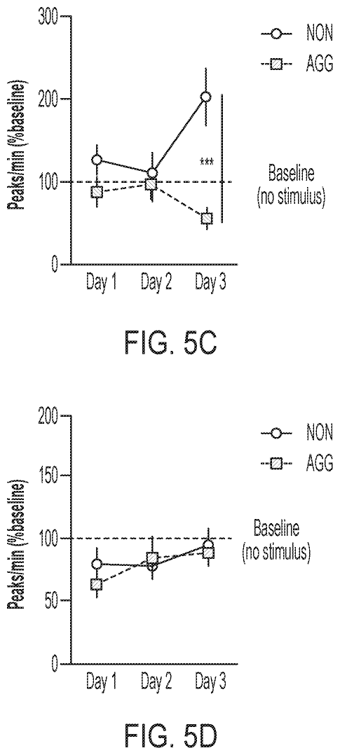

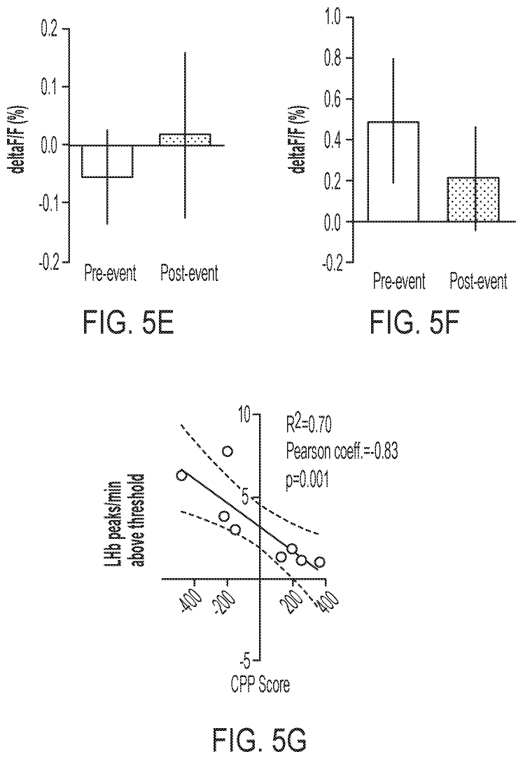

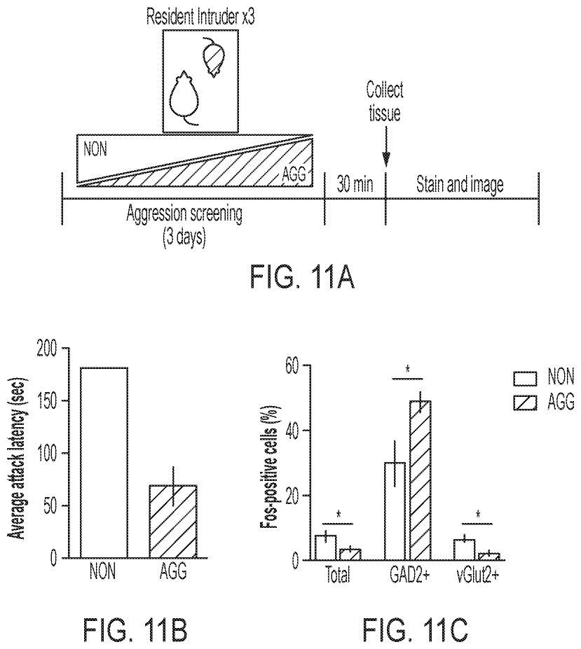

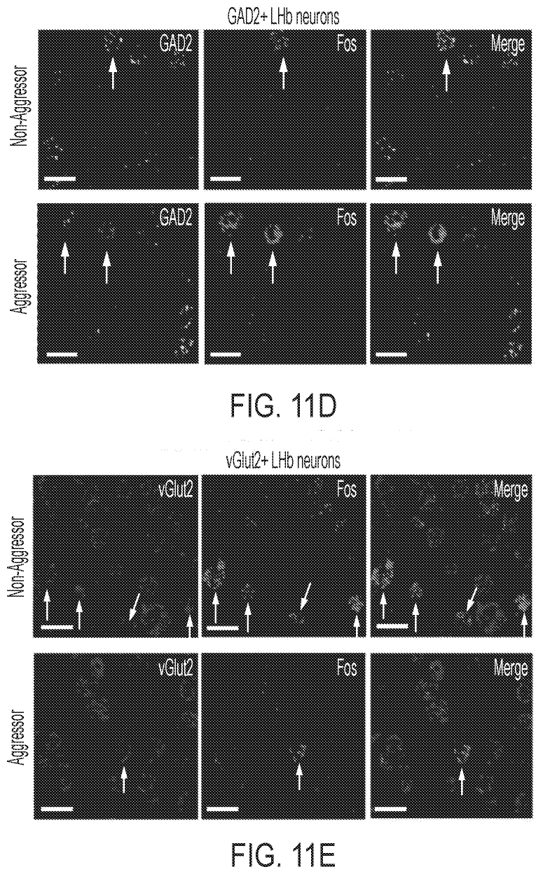

To investigate patterns of LHb activity associated with aggressive social encounters, fiber photometry was used to measure fluorescent calcium transients in highly aggressive (AGG) and non-aggressive (NON) CD-1 outbred mice during the resident intruder (RI) and aggression CPP tests. AAV-hSyn-GCaMP6s were injected into the LHb of wild-type CD-1 mice and implanted a ferrule above the infected cells (FIG. 1A). AGGs displayed lower LHb activity in the presence of a subordinate intruder C57BL6/J mouse than in its absence, while NONs displayed increased LHb activity in the presence of the intruder (FIG. 1A-1D). Notably, these differences were only evident by day 3 of RI, suggesting that repeated winning experience in AGGs or forced non aggressive social interactions in NONs promotes adaptations in LHb responses that reflect the valence of their previous social encounters (FIGS. 5A-5G). Discrete events of the social encounter were then time locked to resulting LHb activity on day 3 of RI, noting an acute decrease in LHb activity in AGGs upon the biting of a submissive intruder (FIG. 1E). NONs, however, displayed increases in LHb activity upon intruder approach (FIG. 1F). AGGs and NONs displayed opposing LHb responses to withdrawal from social bouts, with AGGs showing an increase in activity upon withdrawal from an aggressive social interaction and NONs showing a decrease in activity upon withdrawal from a non-aggressive social interaction (FIGS. 1G-1H). Therefore, the LHb appears to initially serve as a valence detector in AGGs and NONs during early social encounters, but can exhibit plasticity as a consequence of winning fights to result in heightened aggression during future aggressive social encounters. Furthermore, AGGs displayed decreased LHb activity in the paired context compared to the unpaired context during the test phase of CPP (FIG. 11) and CPP scores of AGGs and NONs were both negatively correlated with LHb activity (FIGS. 5A-5G). These data suggest that the LHb encodes the valence of aggressive social interactions.

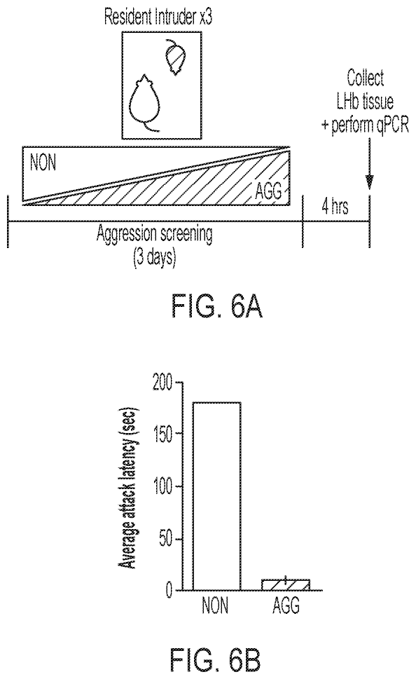

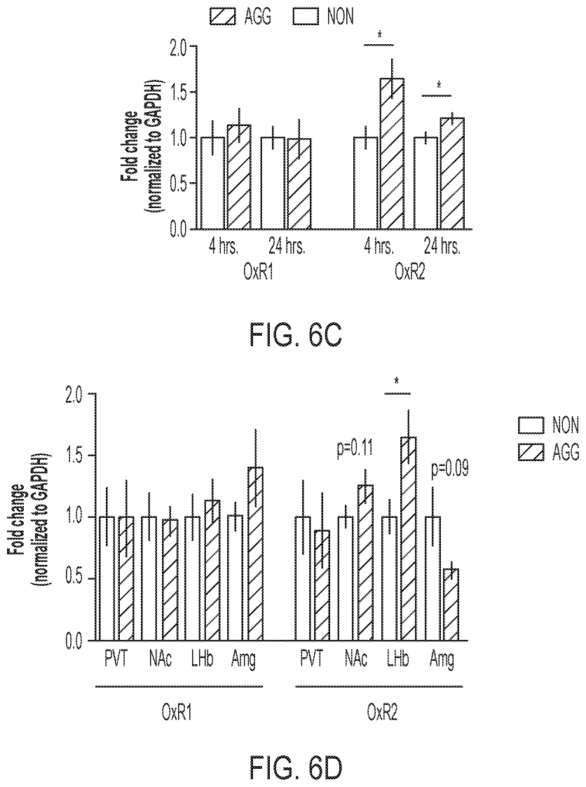

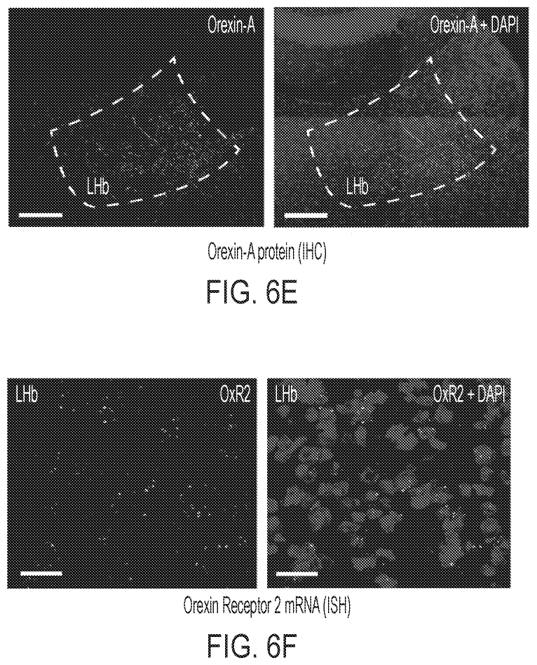

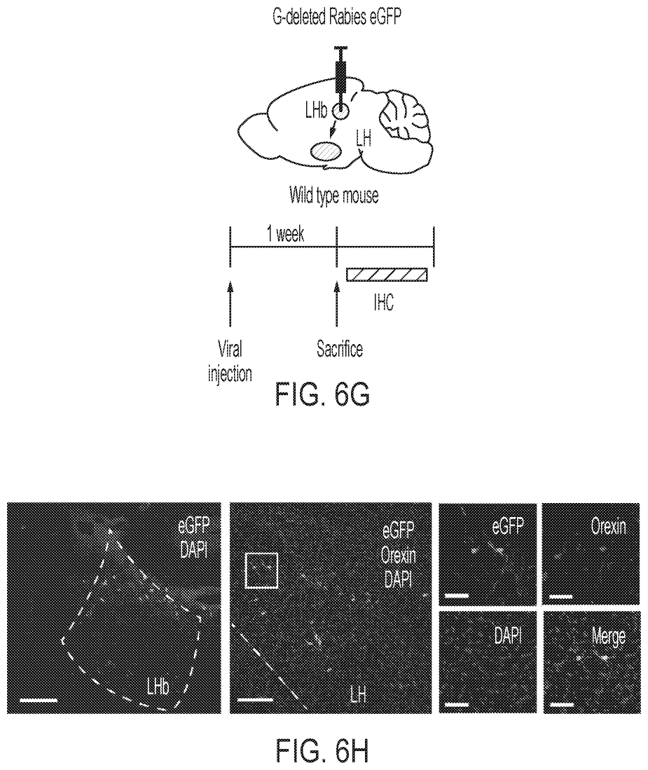

The LH neuropeptide orexin has not been previously implicated in aggressive behavior; however, it has been shown to play important roles in motivation and reward [10]. Using qPCR, it was found that LHb orexin receptor 2 (OxR2) mRNA was upregulated in AGGs compared to NONs following repeated RI (FIGS. 6A-6C). This increase was not observed in nucleus accumbens, amygdala, or paraventricular thalamus (FIG. 6D). Because very little is currently known about the properties of the LH-LHb orexin circuit, histology was utilized to verify the presence of orexin-A positive axons and cell bodies positive for OxR2 mRNA in the LHb (FIGS. 6E-6F). Next, retrograde tracing was performed by injecting a G-deleted rabies eGFP virus in the LHb and stained for orexin-A in the LH (FIGS. 6G-6H). This virus is taken up into presynaptic terminals, but not fibers of passage, and therefore will only infect orexin neurons if they directly synapse onto LHb neurons [11]. Co-localization of orexin-A (red) was observed with GFP (green) in roughly one quarter of all LH-LHb projecting neurons (26.2+3.8%).

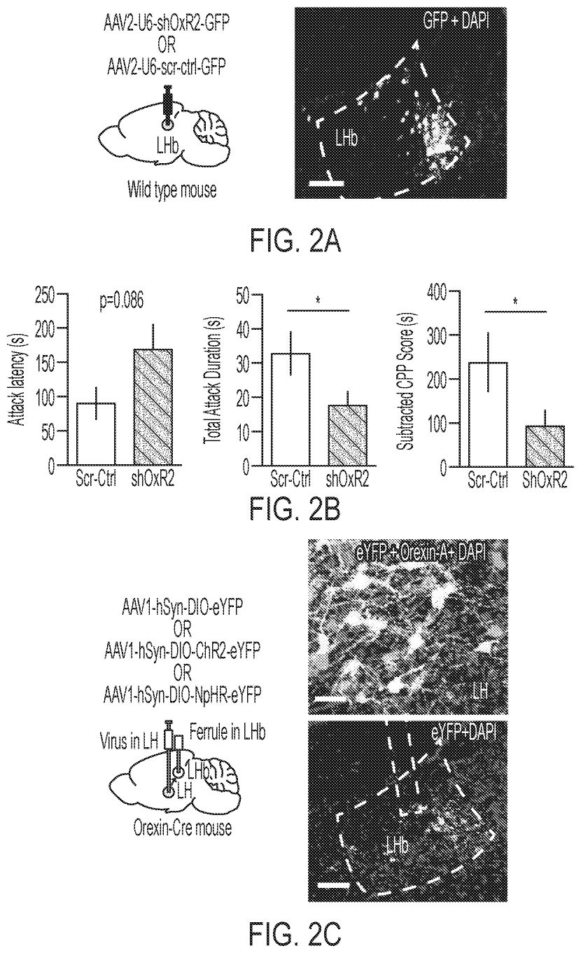

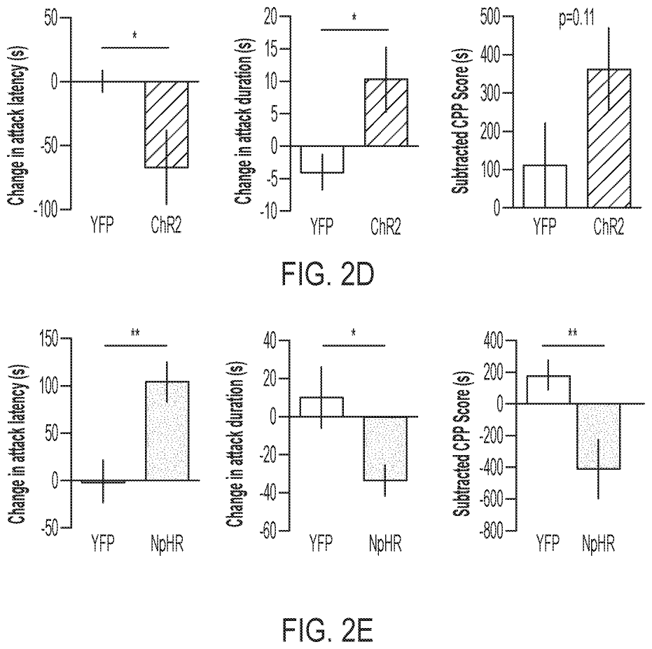

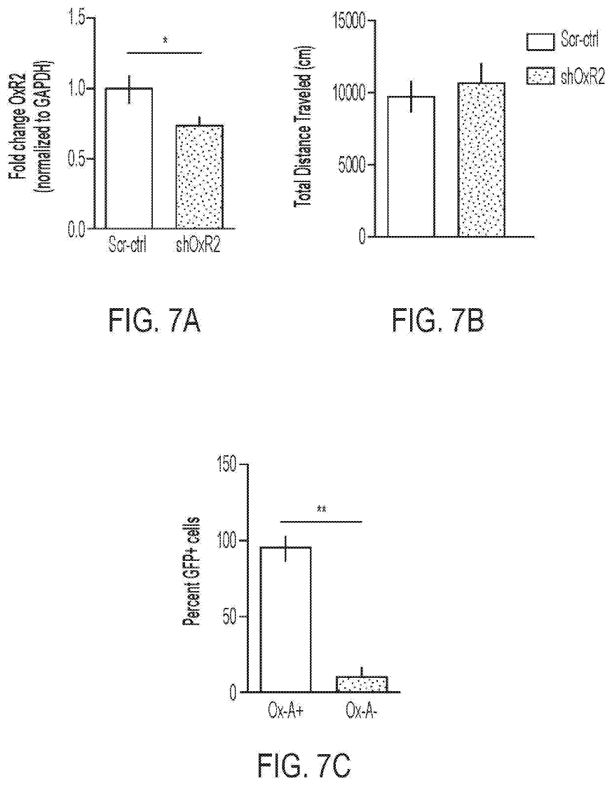

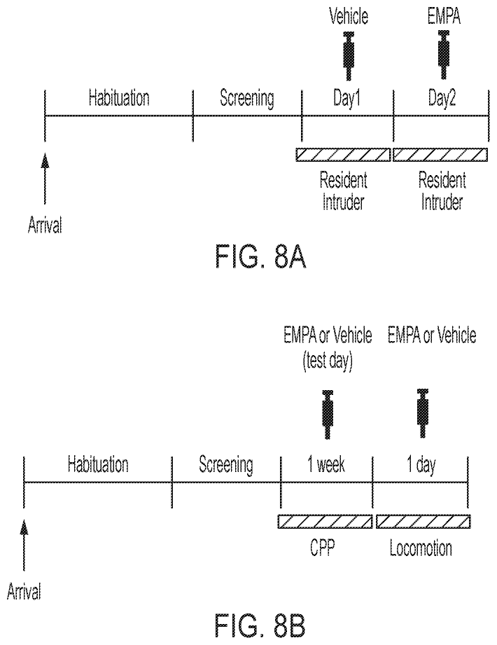

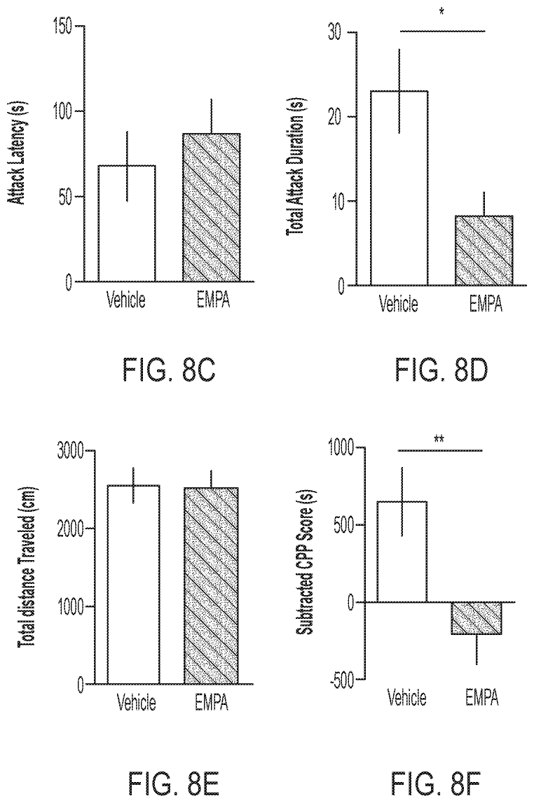

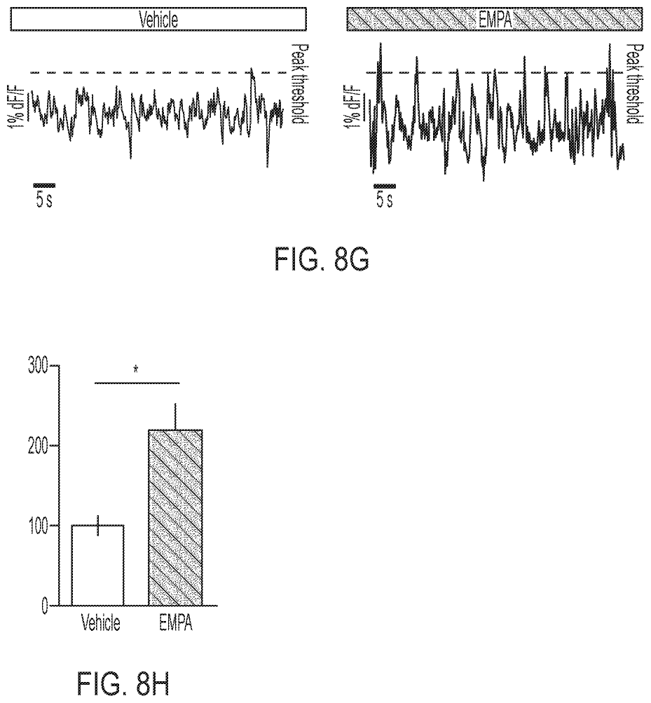

To assess whether LHb orexin signaling is functionally involved in aggression, expression of LHb OxR2 in wild-type AGG mice was non-conditionally inhibited using an AAV-shRNA-mediated strategy (FIGS. 2A, 7A-7C). Inhibition of LHb OxR2 in AGGs decreased attack duration in RI as well as aggression CPP (FIG. 2B). Importantly, experimental and control groups did not differ in their locomotor activity in an open field, indicating the effect was not due to general suppression of activity (FIGS. 7A-7C). ChR2-mediated optogenetic stimulation of orexin terminals in the LHb of AGGs (20 Hz, 20 ms pulses, 7 mW) decreased attack latency and increased total attack duration (FIGS. 2C-2D, FIGS. 7A-7C). In contrast, NpHR-mediated optogenetic inhibition of orexin terminals in the LHb of AGGs increased attack latency and decreased total attack duration in the RI test while also decreasing aggression CPP (FIGS. 2C, 2E). Systemic pharmacological inhibition of OxR2 using the brain-penetrant antagonist N-ethyl-2-((6-methoxy-pyridin-3-yl)-(toluene-2-sulphonyl)-amino)-N-pyridin-3-ylmethyl-acetamide (EMPA, 30 mg/kg) [12] also reduced attack duration and aggression CPP (FIGS. 8A-8H). Together, these results illustrate a previously unknown role for orexin signaling in the LHb in modulating aggressive behavior by altering aggression reward, highlighting the possibility that drugs targeting OxR2 may be useful in reducing the motivation of psychiatric patients to engage in aggressive behavior.

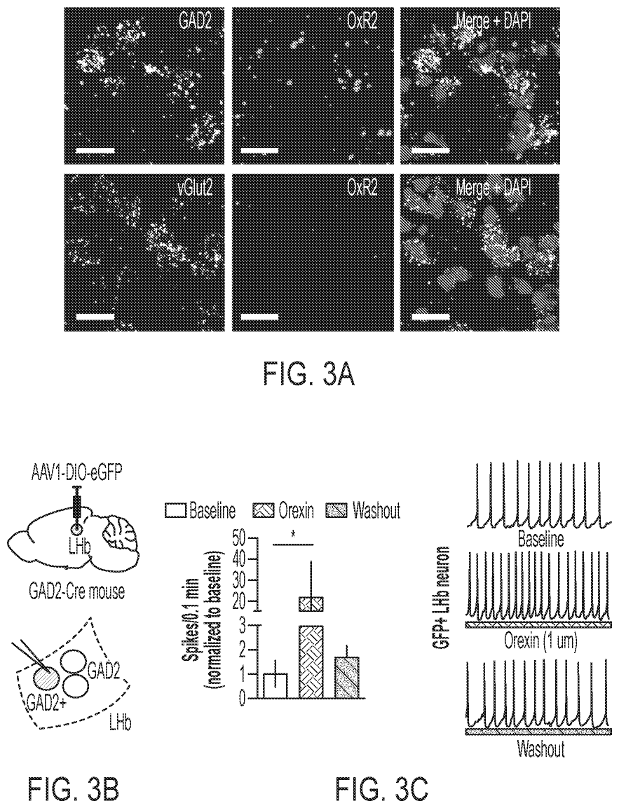

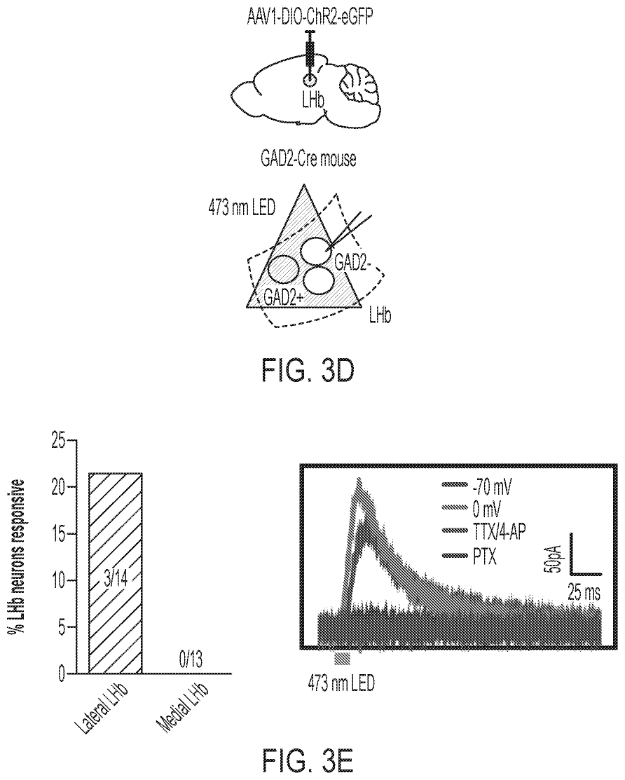

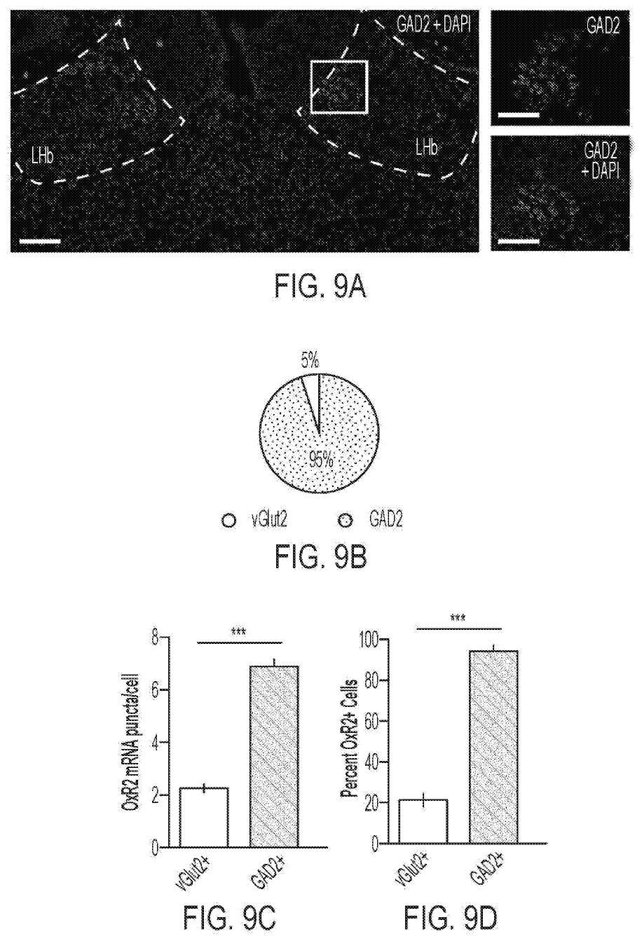

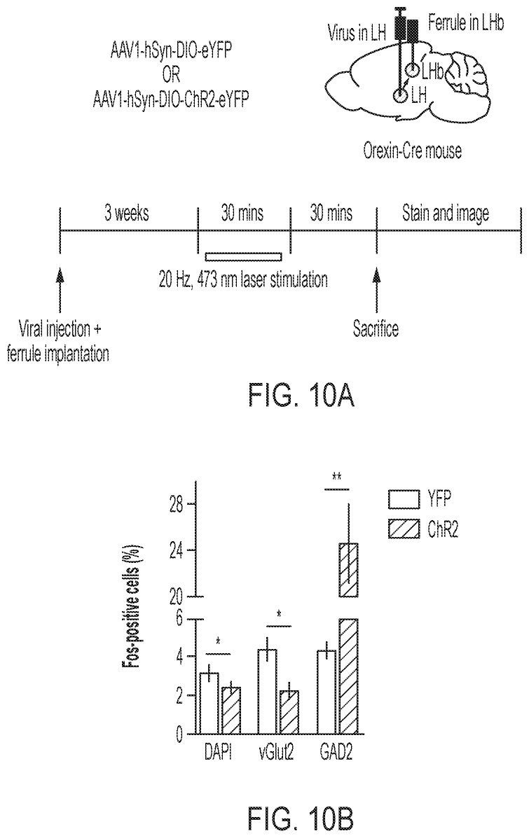

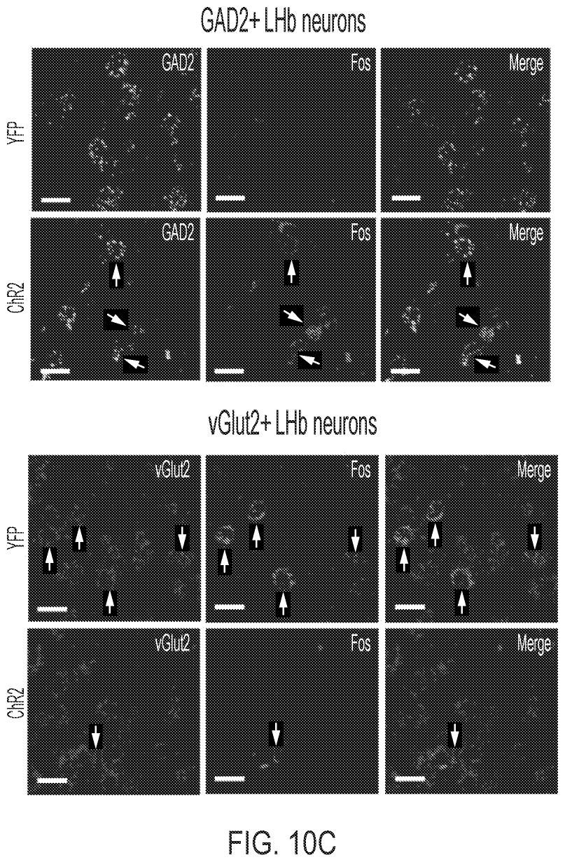

The results indicate that the positive valence of aggressive social encounters is associated with decreased total LHb activity and increased LHb orexin signaling. As orexin receptor activation depolarizes neurons [13], it was predicted that LH orexin neurons synapse on GABAergic neurons in the LHb that subsequently inhibit vGlut2-positive glutamatergic LHb neurons to drive the positive valence of aggression. While some histological evidence suggests there is a small group of GAD2-positive neurons in the LHb, few details are known about the precise anatomy, molecular characteristics, or behavioral function of these neurons [14-16]. Moreover, there is no functional evidence that these cells exhibit interneuron-like properties to drive local inhibition within the LHb. In fact, some have even argued that the LHb is devoid of interneurons [17]. It was found that OxR2 mRNA is highly enriched in GAD2, but not vGlut2, neurons in the LHb (FIG. 3A, FIGS. 9A-9D). These GAD2 neurons comprised ˜5% of total LHb neurons and were primarily localized to the medial division of the LHb (FIGS. 9A-9D). Accordingly, bath application of orexin-A increased the firing rate of GAD2 LHb neurons relative to baseline (FIGS. 3B-3C). In 11% (3/27) of total recorded cells, optogenetic stimulation of LHb GAD2 neurons elicited monosynaptic inhibitory currents in putative glutamatergic LHb neurons (GFP negative) that were completely blocked with the GABA receptor antagonist picrotoxin (FIGS. 3D-3E). These responses were compartmentalized to the lateral (21%, 3/14) rather than medial (0%, 0/13) division of the LHb. In-vivo 20 Hz optogenetic stimulation of orexin terminals in the LHb simultaneously increased LHb GAD2 cell activation and reduced vGlut2 cell activation (FIGS. 10A-10C), mirroring the cell-type specific LHb activation patterns observed in AGGs following RI (FIGS. 11A-11E). Together, these findings support an LH-LHb orexin circuit model whereby orexin activates LHB GAD2 neurons via OxR2 to drive local inhibition of LHb vGlut2 projection neurons.

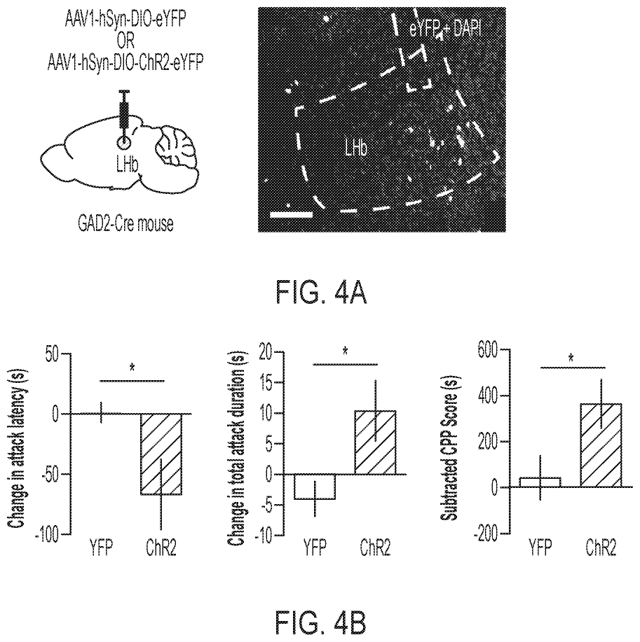

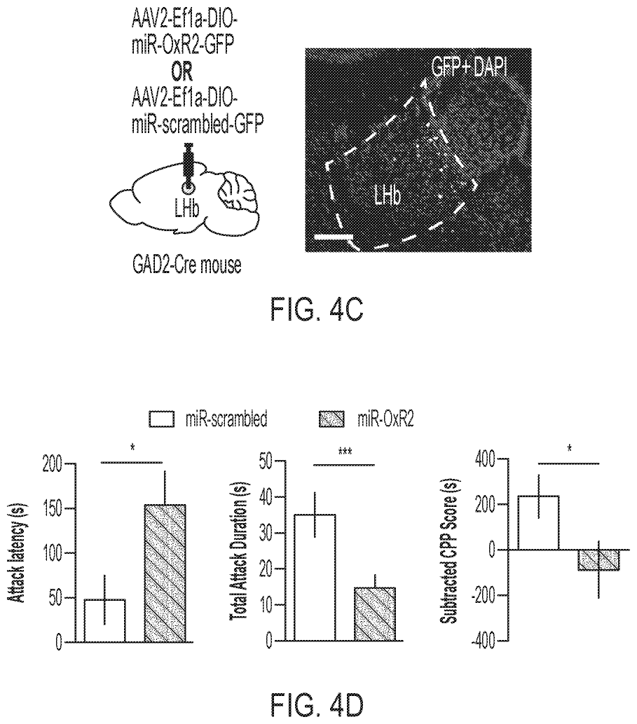

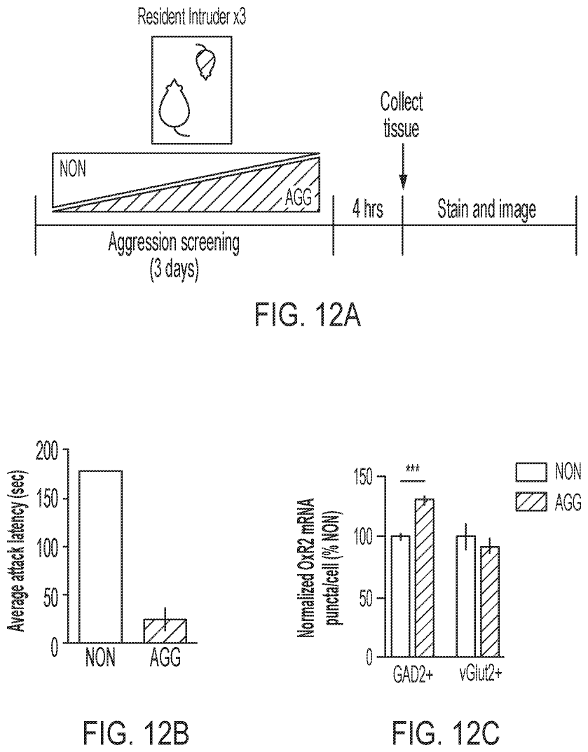

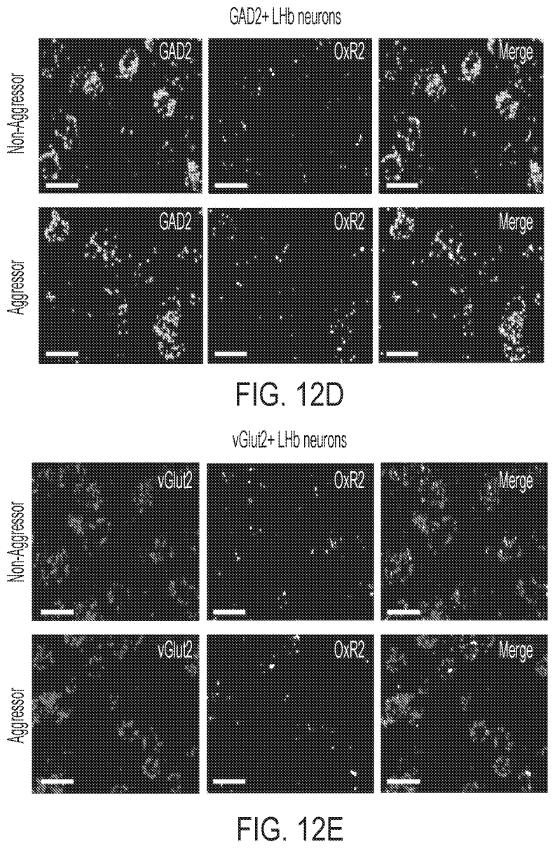

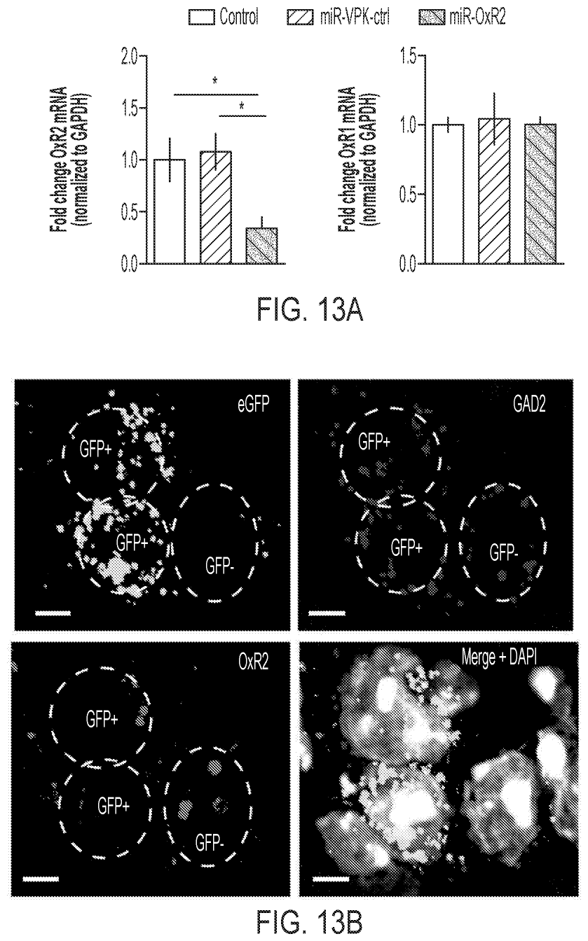

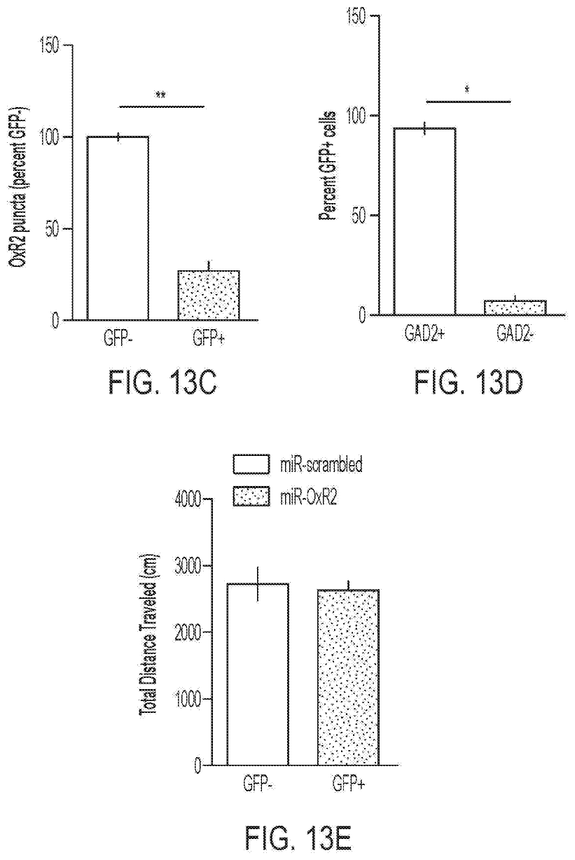

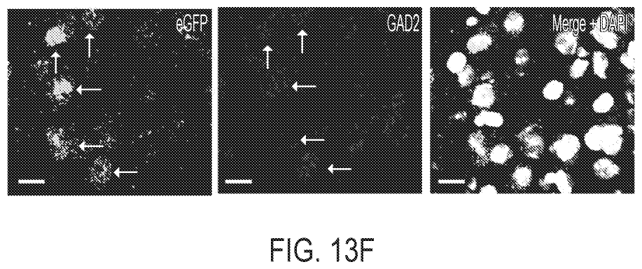

To determine if LHb GAD2 neurons themselves modulate aggression and the valence of aggressive social encounters, they were directly manipulated using optogenetics (FIG. 4A). ChR2-mediated optogenetic excitation of GAD2 LHb neurons during RI (7 mW, 20 Hz, 20 ms pulses) reduced attack latency while increasing total attack duration FIG. 4B). This manipulation also increased aggression CPP (FIG. 4B). It was determined that the observed increase in LHb OxR2 expression with repeated aggression occurs selectively in GAD2 LHb neurons (FIGS. 12A-12E), so next it was tested whether OxR2 expression in LHb GAD2 neurons is necessary for driving the positive valence of aggressive behavior. Knockdown of OxR2 in GAD2 LHb neurons using a miR-based strategy increased attack latency and decreased total attack duration while completely abolishing aggression CPP (FIGS. 4C-4D, FIGS. 12A-12E). This manipulation had no effects on locomotor behavior (FIGS. 13A-13F). These findings support a functional role for OxR2 on LHb GAD2 neurons in promoting local inhibition to enhance aggression and aggression CPP.

Here, the first evidence is provided that orexin is involved in aggression and that GAD2 LHb neurons are capable of providing local inhibitory tone within the excitatory LHb network. It was found that as a result of repeated aggressive experience, orexin-mediated activation of LHb GAD2 neurons via OxR2 reduces the activity of LHb glutamatergic projection neurons in response to aggressive social encounters to promote the positive valence of aggression. The results also indicate that systemic treatment with drugs targeting the orexin system may be effective at curbing aggressive behavior in psychiatric patients. Future studies should aim to determine the specific utility of OxR2 antagonists in treating aggression in diverse patient populations for which pathological aggression is a symptom.

### Materials and Methods

### Animals

For experiments in wild-type animals, 4-month old male CD-1 (ICR) mice (RRID:

IMSR_CRL:22) (sexually experienced retired breeders; Charles River Laboratories (CRL)) were used as subjects. Subjects were confirmed by CRL to have equal access, experience, and success as breeders. For experiments in transgenic animals, C57BL/6J heterozygous Orexin-cre-IRES-GFP (gift from A. Yamanaka, see 17) or homozygous GAD2-Cre (RRID:IMSR_JAX:010802) (Jackson Laboratory) mice were crossed to wild-type CD-1 mice and the F1 generation was used for experiments. This strategy was necessary to ensure a wide range of aggressive phenotypes in experimental animals, as C57BL/6J mice display relatively low levels of aggressive behavior (18). At 3 months of age, F1 transgenic male mice were paired with F1 female mice for two weeks to gain sexual experience before being utilized for experiments. For experiments in transgenic mice, male littermates were randomly assigned to experimental groups. 8-9 week male C57BL/6J mice (RRID: IMSR_JAX:000664) (20-30 g; The Jackson Laboratory) were used as novel intruders. All mice were allowed one week of acclimation to the housing facilities before the start of experiments. Wild-type CD-1 and CD-1/F1 transgenic mice were singly housed and C57BL/6J mouse were housed in groups of 5. All mice were maintained on a 12 h light:dark cycle with ad libitum access to food and water. Procedures were performed in accordance with the National Institutes of Health Guide for Care and Use of Laboratory Animals and the Icahn School of Medicine at Mount Sinai Institutional Animal Care and Use Committee.

### Aggression Screening/Resident Intruder (RI) Test

Aggression screening was performed as previously described by utilizing the resident intruder (RI) test (6). After a minimum of one week of habituation to home cages, experimental mice were exposed to a novel C57BL/6J intruder for 3 min daily over 3 consecutive days. Each intruder presentation was performed in the home cage of the experimental mouse between 12-3 PM daily under white light conditions. During RI sessions the cage top was removed to allow for unobstructed viewing and video recording of sessions. The duration and number of screening sessions were selected to prevent induction of stress- and anxiety-related behaviors in experimental CD-1 or F1 hybrid mice (6). All RI sessions were video recorded with a digital color video camera. Two blind observers recorded (1) the latency to initial aggression and (2) the total duration of aggression. The initiation of aggression was defined by the first clear physical antagonistic interaction initiated by the resident mouse (usually a bite), not including grooming or pursuit behavior. Aggression was considered completed when the resident mouse had reoriented away from the intruder following the initiation of attack. This definition allows for slight breaks (less than 5 s) in continuous physical interaction within an aggressive bout, assuming the resident mouse has remained oriented towards the intruder throughout. Resident mice were defined as AGGs if they initiated aggression during all three screening sessions, while NONs were defined as those that showed no aggression during any screening sessions. Aggression screening was halted if an intruder showed any signs of injury in accordance with the previously published protocol (19).

### Aggression Conditioned Place Preference (CPP)

The aggression CPP protocol was carried out according to Golden et al. (6). Briefly, this task consisted of three phases: pre-test, acquisition (conditioning), and test. Mice were acclimated to the testing facility for 1 h before all testing. All phases were conducted under red light and sound-attenuated conditions. The CPP apparatus (Med Associates) consisted of two unique conditioning chambers with a neutral middle zone that allowed for unbiased entry into either conditioning chamber at the initiation of each trial. During the pre-test phase, mice were placed into the middle chamber of the conditioning apparatus and allowed to freely explore the apparatus for 20 min. There were no group differences in bias for either chamber, and conditioning groups were balanced in an unbiased fashion to account for pre-test preference. The acquisition phase consisted of three consecutive days with two conditioning trials each day for a total of 6 acquisition trials. Morning trials (between 8-10 AM) and afternoon trials (between 3-5 PM) consisted of experimental mice confined to one chamber for 10 min while in the presence or absence of a novel C57BL/6J intruder mouse. All groups were counterbalanced for the conditioning chamber. A total of 3 conditioning trials to the intruder-paired and intruder-unpaired context were performed. On the test day, experimental mice were placed into the middle arena without any intruders and allowed to freely explore the apparatus for 20 min. For optogenetic experiments, light was delivered during the full duration of the test phase only. Total locomotor activity was also recorded to ensure equal exploratory behavior between groups. Behavioral analysis of aggression CPP was performed by calculating (1) CPP score (test phase duration in paired chamber minus test phase duration in unpaired chamber) (2) subtracted CPP score (test phase duration in paired chamber subtracted by pre-test phase duration in paired chamber).

### Open Field Test/Locomotion

The open field test was performed as previously described (20). One week after the last RI, experimental mice were acclimated to the testing facility for 1 h before testing. Open-field tests were performed in black plexiglass arenas (42×42×42 cm; Nationwide Plastics) under red light conditions. Testing sessions lasted for either 5 minutes (GAD2-specific OxR2 knockdown and systemic EMPA experiments) or 10 minutes (non-conditional OxR2 knockdown experiment). Behavior was tracked with Noldus Ethovision (Noldus Interactive Technologies) to record the total distance moved, time spent in the entire arena, and time spent in the delineated “center zone” or “corner zones” of the arena (24×24 cm).

### Behavioral Pharmacology

For EMPA experiments (4558; Tocris), mice were injected interperitoneally (I.P.) with either 30 mg/kg EMPA (Tocris; 4558) dissolved in 0.3% v/v Tween-80 in saline or vehicle (0.3% v/v Tween-80 in saline alone) 25 minutes prior to behavioral testing. For RI tests, animals were given EMPA one day and vehicle the other (counterbalanced for order, within-subjects design). For CPP tests, half of the animals were given EMPA and half were given vehicle (between-subjects design).

### Perfusion and Brain Tissue Processing

For immunohistochemistry and histology, mice were given a lethal dose of 15% chloral hydrate and transcardially perfused with cold PBS (pH 7.4) followed by fixation with cold 4% paraformaldehyde (PFA) in PBS. Brains were dissected and post-fixed for 24 h in 4% PFA. Coronal sections were prepared on a vibratome (Leica) at 50 μm to assess viral placement and perform immunohistochemistry.

For in-situ hybridization, mice were rapidly decapitated and brains were removed and flash frozen in −30 degrees C. isopentane for 30 s and then stored at −80 degrees C. until sectioning. Coronal sections for in-situ were prepared on a cryostat at 16 μm thickness and mounted directly on slides.

For real-time quantitative PCR (RTqPCR), mice were rapidly decapitated and the brains were extracted and placed in ice-cold PBS. Bilateral LHb 1 mm diameter, 1 mm thick, tissue punches were taken and immediately flash frozen on dry ice and stored at −80 degrees until RNA extraction.

RNA Extraction, Generation of cDNA, and RTqPCR

RNA was isolated from either brain tissue or HEK293 cells using TRIzol (Invitrogen) homogenization and chloroform layer separation. The clear RNA layer was processed with the RNAeasy MicroKit (Qiagen), analyzed with the NanoDrop (Thermo Fisher Scientific), and 500 ng of RNA was reverse transcribed to cDNA with qScript (95048-500; Quanta Biosciences). The resulting cDNA was diluted to 1 ng/μl. For each reaction, 3 μl of cDNA was combined with 5 μl of Perfecta SYBR Green (95054-02K; Quanta Biosciences), forward/reverse primers (1 μl total), and 1 μl of water. Samples were heated to 95 degrees C. for 2 min followed by 40 cycles of 95 degrees C. for 15 s, 60 degrees C. for 33 s, and 72 degrees C. for 33 s. Analysis was performed using the delta_deltaC(t) method. Samples were normalized to GAPDH. Primers used were as follows: GAPDH (F: AAC GGC ATT GTG GAA GG, R:GGA TGC AGG GAT GAT GTT CT), orexin receptor 1 (OxR1) (F: ATC CAC CCA CTG TTG TT, R: GGC CAG GTA GGT GAC AAT GA), orexin receptor 2 (OxR2) (F: CAT CGT TGT CAT CTG GAT CG, R: GGC ACC AGA GTT TAC GGA AT).

### Generation and Validation of AAV2 Cre-Dependent OxR2 Viral Constructs

To create an effective Cre-dependent knockdown virus for OxR2, a micro-RNA (miR) based approach was utilized. The miR was bicistronically expressed with IRES-eGFP for simple identification of infected cells. Briefly, the OxR2 miR was generated using the BLOCK-iT™ Pol II miR RNAi expression vector kit (Thermo Fisher Scientific). The shRNA sequence was used from the non-conditional OxR2 virus, which was previously validated to inhibit OxR2 expression (Arendt et al. 2014). A scrambled sequence was used as the control. The miR-containing oligonucleotides were inserted into a pcDNA6.2-GW-miR vector provided by the kit. The miR sequences, along with the 5′ and 3′ flanking regions, were then sub-cloned into a bicistronic IRES-GFP vector (pAAV-IRES-GFP, Cell Biolabs). This vector was non-conditional and can be expressed in mammalian cells. Suppression of OxR2 with the miR construct was validated in HEK293 cells by RTqPCR (see above). Once validated in-vitro, the miR-IRES-GFP sequence was inserted into a Cre-dependent AAV2.Ef1a.DIO vector and packaged (Virovek Inc.) to produce AAV2.Ef1a.DIO.miROxR2.IRES.GFP.SV40pA and pAAV.Ef1a.DIO.miRscrambled.IRES.GFP.SV40pA. These constructs were validated in-vivo through injection into GAD2-cre F1 mice and subsequent in-situ hybridization for GFP, GAD2, and OxR2.

### Immunohistochemistry, In-Situ Hybridization, and Confocal Microscopy

For immunohistochemistry experiments, sections were incubated overnight in blocking solution (3% normal donkey serum, 0.3% Triton X-100 in PBS), washed three times in PBS for 10 min (30 min total), then incubated for 24 h in primary antibodies diluted in blocking solution (goat anti-Orexin-A (Santa Cruz Biotechnology, sc-8070; 1:500); chicken anti-GFP (Aves Labs, GFP-1020; 1:1000). Sections were then washed three times in PBS for 10 min (30 min total), incubated for 2 h in secondary antibodies diluted in blocking solution (donkey anti-goat Cy3 1:400 (Jackson ImmunoResearch; 705-165-003); donkey anti-chicken AlexaFluor 488 1:400 (Jackson ImmunoResearch; 703-545-155), and washed three times in PBS for 10 min (30 min total). Finally, sections were counterstained with 1 ug/ml DAPI (Sigma) for 10 min and mounted on slides. Sections were allowed to dry on slides overnight, dehydrated with ethanol and then Citrisolv, and cover-slipped with ProLong Diamond Antifade Mountant (Invitrogen; P36970). Immunohistochemistry was imaged with a LSM 780 confocal microscope (Carl Zeiss) at either 10× or 20× magnification and analyzed with FIJI (ImageJ) software.

For in-situ hybridization experiments, the RNAScope Multiplex Fluorescent in-situ kit (Advanced Cell Diagnostics) was utilized according to the manufacturer's instructions. Briefly, fresh frozen sections were fixed in ice-cold 4% PFA in PBS for 15 minutes, serially dehydrated with EtOH (50%, 75%, 100%; each for 2 minutes), and pretreated with a protease (Protease IV, RNAScope) for 30 min. Proprietary probes for eGFP, glutamic acid decarboxylase 2 (GAD2), or vesicular glutamate transporter 2 (vGlut2), or orexin receptor 2 (OxR2) probes were hybridized at 40 degrees C. for 2 h, serially amplified, counterstained with 1 ug/ml DAPI for 2 min, and immediately cover-slipped with EcoMount mountant (Biocare Medical). In-situ hybridization was imaged with a Zeiss LSM 780 confocal microscope at 20×, 40×, or 63× magnification. mRNA puncta were quantified manually using FIJI software (Image J).

### In-Vitro Electrophysiology

Slice Preparation

Adult male mice were anesthetized with isoflurance, decapitated, and the brain was immediately removed and submerged into ice-cold sucrose-artificial CSF (aCSF) comprising (in mM) 233.7 sucrose, 26 NaHCO₃, 3 KCl, 8 MgCl₂, 0.5 CaCl₂), 20 glucose, and 0.4 ascorbic acid. Coronal sections (350 microns thick) were sliced using a Leica VT1000S vibratome and allowed to equilibrate in recording aCSF (in mM: 117 NaCl, 4.7 KCl, 1.2 MgSO₄, 2.5 CaCl₂), 1.2 NaH₂PO₄, 24.9 NaHCO₃, and 11.5 glucose) oxygenated with 95% O₂-5% CO₂ for 1 hour at RT. Slices were then transferred to the recording chamber where they were maintained at 31 degC and perfused (1.5 ml/min) with oxygenated aCSF.

Orexin Bath Application Recordings

2 weeks prior to recording, GAD2-cre were injected in the lateral habenula (see coordinates in following section) with AAV1.hSyn.DIO.eYFP (UPenn Viral Core). Mice were then returned to their homecage to allow for viral expression before being killed for slice recordings. During recording, cells were identified as GAD2-positive by expression of eYFP in the lateral habenula. Neurons were visualized on an upright epifluorescence microscope (BX50WI; Olympus) with a 40× water-immersion objective and an infrared CCD monochrome video camera (Dage-MTI). Whole-cell recordings were performed with glass micropipettes (resistance 2-4 MΩ) pulled from borosilicate glass capillaries using a P-87 micropipette puller (Sutter Instruments). The pipettes were filled with an intracellular solution containing: 124 mm K-gluconate, 10 mm HEPES, 10 mm phosphocreatine di(Tris), 0.2 mm EGTA, 4 mm Mg2ATP, and 0.3 mm Na2GTP, adjusted to an osmolarity of 280-290 mOsm and pH of 7.3. Recordings were made with a Multiclamp 700B (Molecular Devices) in current-clamp mode. Analog signals were low-pass filtered at 2 kHz and digitized at 5 kHz using a Digidata 1440A interface and pClamp10 software (Molecular Devices). Gigaseal and access to the intracellular neuronal compartment was achieved in voltage-clamp mode, with the holding potential set at −70 mV. After rupturing the membrane, intracellular neuronal fluid equilibrated with the pipette solution without significant changes in series resistance or membrane capacitance. Cells were allowed to normalize for 5 minutes before recording. To test response of cells to orexin A (Phoenix Pharmaceuticals), orexin A (1 μM) was applied for 5 minutes in the circulating bath after a 2-minute baseline period, followed by washout for 45-60 minutes. Orexin A was prepared freshly and dissolved in aCSF before being added to recording bath.

Offline analysis was performed using Clampfit (Molecular Devices). Spikes were counted during baseline, orexin washin-in, and wash-out periods. To account for bath circulation, spikes were counted 2 minutes after drug was added or after wash-out began. Kruskal-Wallis test with Dunn's post-hoc tests for multiple comparisons was conducted to analyze mean firing rate between groups (baseline, orexin wash-in, and wash-out) using GraphPad Software Prism (version 5.01).

Slice Optogenetic Stimulation of LHb GAD2 Neurons Recording

Recordings were obtained with borosilicate glass electrodes (5-8 MΩ resistance) filled with voltage clamp internal solution (in mM: 120 Cs-methanesulfonate, 10 HEPES, 10 Na-phosphocreatine, 8 NaCl, 5 TEA-Cl, 4 Mg-ATP, 1 QX-314, 0.5 EGTA, and 0.4 Na-GTP). Cells were visualized on an upright DIC microscope equipped with a 460 nm objective-coupled LED (Prizmatix, Givat-Shmuel, Israel) for verification of ChR2 expression as well as optogenetic cellular manipulations. Data were low-pass filtered at 3 kHz and acquired at 10 kHz using Multiclamp 700B and pClamp 10 (Molecular Devices, Sunnyvale, Calif., USA). The polarity of light-evoked stimulation (λ 460, 1 mW, 1-5 ms) of ChR2+ terminals was determined by clamping cells at −70 mV (excitatory responses) and 0 mV (inhibitory responses). The monosynaptic nature of light-evoked currents was confirmed by bath application of tetrodotoxin (1 μM; Abcam, Cambridge, Mass., USA) and 4-aminopyridine (100 μM; Abcam) as previously described (21-22). The location of cells within the LHb was confirmed visually after recording.

Stereotaxic Surgery and Viral Gene Transfer

Mice were anesthetized with a mixture of ketamine (100 mg/kg body weight) and xylazine (10 mg/kg body weight) and placed securely in a stereotaxic frame (David Kopf Instruments). 33-gauge syringes (Hamilton Co.) were used to bilaterally infuse 0.5 μl of virus over 5 min and virus was allowed to diffuse for 5 minutes before the needle was withdrawn. For lateral hypothalamus (LH) injections, the coordinates from bregma were −1.3 mm AP, +/−1.1 mm ML, and −5.1 mm DV at a 0-degree angle. For lateral habenula (LHb) injections, the coordinates from bregma were −1.7 mm AP, +/−0.6 mm ML, and −2.7 mm DV at a 10-degree angle. For all optogenetics experiments animals were implanted with an optical fiber at the same time as viral injection (−2.3 mm DV). Optic fibers (Doric Lenses, MFC_200/240-0.22_2.6 mm_FLT) were secured to the skull using C&B Metabond adhesive luting cement (Parkell Dental Supply). For electrophysiology experiments, GAD2-cre F1 mice were injected bilaterally in the LHb with either AAV1-hSyn-DIO-eYFP or AAV1-hSyn-DIO-ChR2.eYFP (UPenn Viral Core). For G-deleted rabies experiments, EnvA G-deleted Rabies-eGFP (Salk Institute Gene Transfer, Targeting, and Therapeutics Core) was injected unilaterally into the LHb of wild-type CD-1 mice. For orexin optogenetics experiments, AAV1-hSyn-DIO-NpHr3.0-eYFP, AAV1-hSyn-DIO-ChR2-eYFP, or AAV1-hSyn-DIO-eYFP (UPenn Viral Core) was injected into the LH of Orexin-cre-IRES-GFP F1 mice and the optic fiber was placed in the LHb. Importantly, IRES-GFP labeling of orexin neurons is not visible without enhancement of GFP signal using immunohistochemistry, while viral eYFP is visible in perfused slices without amplification. This is evident in images of LH tissue from orexin-cre animals injected with AAV-DIO-eYFP in FIG. 3F-only a portion of the total orexin neurons are green due to infection with the virus. The specificity of viral expression in orexin positive neurons was confirmed (FIG. 6E). For GAD2 neuron optogenetics experiments, GAD2-cre F1 mice were injected bilaterally in the LHb with either AAV1-hSyn.DIO-NpHr3.0-eYFP, AAV1-hSyn-DIO-ChR2-eYFP, or AAV1-hSyn-DIO-eYFP (UPenn Viral Core). For non-conditional knockdown of OxR2, wild-type CD-1 mice were injected bilaterally in the LHb with either AAV2-U6-shOxR2 (shRNA targeting OxR2) or AAV2-U6-sh-scrambled (scrambled shRNA sequence). These constructs were validated in-vivo using RTqPCR for OxR2 from brain tissue injected with the virus and validated in a previous publication (Arendt et al. 2014) (shOxR2 sequence: AGAAACCCTTCAGTGGGACTTAAC (SEQ ID NO.: 1), scrambled sequence: CGGAATTTAGAAACCCGGCTCCAC (SEQ ID NO.: 2)). For conditional knockdown and over-expression of OxR2, GAD2-cre F1 mice were injected bilaterally in the LHb with either AAV2-Ef1a-DIO-miROxR2-IRES-eGFP (knockdown) or AAV2-Ef1a-DIO-eGFP (Virovek, Inc, generated for this paper). For fiber photometry experiments, wild-type CD1 mice were injected unilaterally in the LHb with AAV1-hSyn-GCaMP6s. Optic fibers for photometry (Doric Lenses, MFC_400/430-0.48_2.7 mm_MF2.5-FLT) were implanted over the LHb at the same time as viral injection (DV—2.6 mm) and secured with cement. AAV1 viruses were allowed 2-4 weeks for expression, AAV2 viruses were allowed 4-6 weeks for expression, and EnvA G-deleted Rabies-eGFP was allowed 1 week for expression (sacrificed one week following injection).

Optogenetic Stimulation

For blue light stimulation (ChR2), optical fibers (Doric Lenses, MFC_200/240-0.22_2.6 mm_FLT) were connected to a 473-nm blue laser diode (Crystal Laser, BCL-473-050-M) using a patch cord with an FC/PC adapter (Doric Lenses, MFP_200/240/900-0.22_4m_FC-MF2.5). A function generator (Agilent Technologies; 33220A) was used to generate 20 ms blue light pulses at 20 Hz. The intensity of light delivered to the brain was 7-10 mW. These parameters are consistent with previously validated and published protocols (6). In particular, 20 Hz was selected for excitation of orexin neurons since in-vitro experiments have demonstrated that 20 Hz stimulation is sufficient to elicit activation of orexin receptors (23).

For yellow light stimulation (NpHR), optical fibers were connected to a 561-nm yellow laser diode (Crystal Laser, CL561-050L) using an FC/PC adapter. A function generator (Agilent Technologies; 33220A) was used to generate constant light pulses for 8 s followed by 2 s of light off. The intensity of the light delivered to the brain was 7-10 mW. These parameters are consistent with previously validated and published protocols (6).

For all optogenetics experiments, experimental mice were habituated to patch cords for 5 days prior to testing in RI and aggression CPP. For RI experiments, mice were tested twice in the same day (in a counterbalanced fashion) in both laser on and laser off conditions with at least 4 hours between sessions (within subjects design). For CPP experiments, all groups received laser stimulation (between subjects design).

### Fiber Photometry

Apparatus

A fiber optic patch cord (Doric Lenses, MFP_400/430/1100-0.37_3m_FC-MF2.5) was attached to the implanted fiber optic cannula with cubic zirconia sleeves. In turn, the fiber optic cable was coupled to the apparatus for light delivery and signal measurement. GCaMP6s signal was measured by passing 490 nm LED light (Thorlabs) through a GFP excitation filter (MF469; Thorlabs) and dichroic mirrors (DMLP425, MD498; Thorlabs) into the brain and focusing emitted light onto a photodetector (2151 femtowatt receiver; Newport) after passing it back through a dichroic mirror (MD498; Thorlabs), through a GFP emission filter (MF525-39; Thorlabs), and through a 0.50 N.A. microscope lens (62-561; Edmund Optics). To account for auto-fluorescence and possible motion artifacts during testing, a second 405 nm LED not corresponding to GCaMP6s delivered light through a violet excitation filter (FB405-10; Thorlabs) and the same dichroic mirrors as the 490 nm light. This signal was similarly directed into the brain and subsequently measured with the photodetector. Light at the fiber tip ranged from 30 to 75 μW but was constant across trials over days. Simultaneous recording of both 490 and 405 nm channels was achieved through sinusoidal modulation of the LEDs at different frequencies so that the signals could be easily unmixed. Signals were collected at a rate of 381 Hz and visualized using a real-time signal processor (RX8; Tucker-Davis Technologies) and PC OpenEx software.

Behavior

Mice were habituated to the recording apparatus and patch cord for three days prior to all experiments. The timeline for photometry experiments was as follows: viral injection and ferrule implantation (day 0), habituation to patch cord (days 14-15), RI recordings (days 16-18), and CPP recordings (days 21-25). Once hooked up to the apparatus, mice were allowed to rest for 10 minutes before the start of recording. Once recording was initiated, the GCaMP signal was allowed to stabilize for two minutes before the start of the behavioral trial. During RI testing days 5 minutes of baseline recordings and 5 minutes of novel object exposure were collected while the animal was in its home cage for relative comparison to the 5 minute RI session. For CPP, photometry data was collected during the entire 20-minute pre-test and test.

Analysis

Analysis of signals was done using custom written MATLAB code (24). The bulk fluorescent signal from each channel was normalized to compare across recording sessions and animals. Change in fluorescence was calculated as a percentage of the total fluorescence signal in the GCaMP channel (deltaF/F). The 405 channel served as the control channel and was subtracted from the GCaMP channel to eliminate signals due to auto-fluorescence, bleaching, and the bending of the fiber optic cord during aggression. In general, these motion artifacts had very minimal effects on the overall GCaMP signal. Behavioral data was temporally aligned with fluorescence recording data by sending 1s-interval TTL signals to the OpenEx software from Noldus Ethovision (Noldus Interactive Technologies) behavioral recording software. To identify peak signals, the median average deviation (MAD) of the corrected/normalized data sets was first determined. Peak events that exceeded the MAD by 2.91 deviations were determined to be significant peaks, and this is in accordance with previous reports using the fiber photometry technique (24). For analysis of LHb GCaMP activity during discrete behaviors in RI, average deltaF/F signals (%) in the two seconds before and after a discrete event (bite, approach, withdrawal) were compared. A bite was determined to occur at the moment of jaw closing on the body of the intruder, an approach at the moment of resident nose contact with any body part of the intruder, and a withdrawal at the moment body contact between resident and intruder ceased and one or both mice turned away from the site of interaction.

### Statistics

All statistical details can be found in the figure legends, including type of statistical analysis used, p values, n, what n represents, degrees of freedom, and t or F values. For comparisons of two experimental groups, either paired (within subject) or unpaired (between subject) t-tests were used. For parametric data sets, comparisons of three or more groups were performed using one or two-way ANOVA tests followed by either Bonferoni or Neuman-Keulls posthoc tests. For non-parametric data sets, comparisons of three or more groups were performed using Kruskall-Wallis one-way ANOVA followed by Dunn's test for multiple comparisons. For all tests, p<0.05 was deemed significant. Statistical analyses were performed using Graph Pad Prism 5 software (RRID: SCR-002798).

### REFERENCES (EXAMPLE 1)

1. Barlow, K., B. Grenyer, and O. Ilkiw-Lavalle, Prevalence and precipitants of aggression in psychiatric inpatient units. Aust N Z J Psychiatry, 2000. 34(6): p. 967-74.
2. Nell, V., Cruelty's rewards: the gratifications of perpetrators and spectators. Behav Brain Sci, 2006. 29(3): p. 211-24; discussion 224-57.
3. Couppis, M. H. and C. H. Kennedy, The rewarding effect of aggression is reduced by nucleus accumbens dopamine receptor antagonism in mice. Psychopharmacology (Berl), 2008. 197(3): p. 449-56.
4. Falkner, A. L., et al., Hypothalamic control of male aggression-seeking behavior. Nat Neurosci, 2016. 19(4): p. 596-604.
5. Golden, S. A., et al., Compulsive Addiction-like Aggressive Behavior in Mice. Biol Psychiatry, 2017. 82(4): p. 239-248.
6. Golden, S. A., et al., Basal forebrain projections to the lateral habenula modulate aggression reward. Nature, 2016. 534(7609): p. 688-92.
7. Gonzalez, J. A., et al., Awake dynamics and brain-wide direct inputs of hypothalamic MCH and orexin networks. Nat Commun, 2016. 7: p. 11395.
8. Lin, D., et al., Functional identification of an aggression locus in the mouse hypothalamus. Nature, 2011. 470(7333): p. 221-6.
9. Chou, M. Y., et al., Social conflict resolution regulated by two dorsal habenular subregions in zebrafish. Science, 2016. 352(6281): p. 87-90.
10. Sakurai, T., The role of orexin in motivated behaviours. Nat Rev Neurosci, 2014. 15(11): p. 719-31.
11. Osakada, F. and E. M. Callaway, Design and generation of recombinant rabies virus vectors. Nat Protoc, 2013. 8(8): p. 1583-601.
12. Malherbe, P., et al., Biochemical and behavioural characterization of EMPA, a novel high-affinity, selective antagonist for the OX(2) receptor. Br J Pharmacol, 2009. 156(8): p. 1326-41.
13. Kukkonen, J. P. and C. S. Leonard, Orexin/hypocretin receptor signalling cascades. Br J Pharmacol, 2014. 171(2): p. 314-31.
14. Geisler, S., K. H. Andres, and R. W. Veh, Morphologic and cytochemical criteria for the identification and delineation of individual subnuclei within the lateral habenular complex of the rat. J Comp Neurol, 2003. 458(1): p. 78-97.
15. Weiss, T. and R. W. Veh, Morphological and electrophysiological characteristics of neurons within identified subnuclei of the lateral habenula in rat brain slices. Neuroscience, 2011. 172: p. 74-93.
16. Zhang, L., et al., A GABAergic cell type in the lateral habenula links hypothalamic homeostatic and midbrain motivation circuits with sex steroid signaling. Transl Psychiatry, 2018. 8(1): p. 50.
17. Meye, F. J., et al., Synaptic and cellular profile of neurons in the lateral habenula. Front Hum Neurosci, 2013. 7.
18. Tsunematsu, T., et al., Acute Optogenetic Silencing of Orexin/Hypocretin Neurons Induces Slow-Wave Sleep in Mice. J Neurosci, 2011. 31(29).
19. Golden, S. A., et al., Persistent conditioned place preference to aggression experience in adult male sexually-experienced CD-1 mice. Genes Brain Behav, 2016.
20. Golden, S. A., et al., A standardized protocol for repeated social defeat stress in mice. Nat Protoc, 2011. 6(8): p. 1183-91.
21. Krishnan, V., et al., Molecular adaptations underlying susceptibility and resistance to social defeat in brain reward regions. Cell, 2007. 131(2): p. 391-404.
22. Arruda-Carvalho, M. and R. L. Clem, Pathway-selective adjustment of prefrontal-amygdala transmission during fear encoding. J Neurosci, 2014. 34(47): p. 15601-9.
23. Petreanu, L., et al., The subcellular organization of neocortical excitatory connections. Nature, 2009. 457(7233): p. 1142-5.
24. Schöne, C. and et al., Coreleased orexin and glutamate evoke nonredundant spike outputs and computations in histamine neurons.—PubMed—NCBI. 2014.
25. Calipari, E. S., et al., In vivo imaging identifies temporal signature of D1 and D2 medium spiny neurons in cocaine reward. Proc Natl Acad Sci USA, 2016. 113(10): p. 2726-31.

### Example 2: Systemic Antagonism of Orexin Receptors Reduces Aggressive Behavior

### Abstract

Orexin (hypocretin) is a hypothalamic neuropeptide that has been implicated in a wide variety of reward and arousal-related behaviors ranging from motivation for addictive drugs to freezing responses to aversive stimuli. It was recently found that systemic treatment with the brain-penetrant orexin receptor 2 (OxR2) antagonist N-ethyl-2-[(6-methoxy-pyridin-3-yl)-(toluene-2-sulphonyl)-amino]-N-pyridin-3-ylmethyl-acetamide (EMPA) reduces aggression by blunting the reinforcing properties of winning fights. However, it is unknown whether selective antagonism of orexin receptor 1 (OxR1) or dual antagonism of both orexin receptors is also effective in reducing aggression. To investigate this, a variety of OxR2, OxR1, and dual receptor antagonists were systemically administered and aggression, locomotor activity, and anxiety behavior were assessed in male mice. It was found that while selective antagonism of OxR2 produced the strongest reduction in aggressive behavior, selective antagonism of OxR1 was also moderately effective. Dual antagonism of orexin receptors was only effective at reducing aggression following chronic treatment. None of the orexin-targeting drugs produced locomotor deficits or altered anxiety at the dosages tested. The results of this study suggest that drugs selectively targeting OxR2 are promising candidates for reducing aggressive behavior in psychiatric patient populations.

### Introduction

Interpersonal aggression and violence is a prevalent symptom of many neuropsychiatric disorders including but not limited to drug addiction (Beck et al., 2014; Coccaro et al., 2016), autism (Fitzpatrick et al., 2016), depression (Dolenc et al., 2015), antisocial personality disorder (Anderson and Kiehl, 2014), PTSD (Miles et al., 2016), and schizophrenia (Hoptman, 2015). In addition to producing a variety of negative social and physical consequences for victims and patients, those who experience or witness violence display increased risk for developing neuropsychiatric disorders like PTSD and depression (Sumner et al., 2015). While clearly a tremendous public health issue, there are currently no approved treatments aimed at reducing aggression in psychiatric patients. Selective serotonin reuptake inhibitors, benzodiazepenes, and anti-psychotics are widely prescribed off-label to treat aggression, but the efficacy of these drugs is extremely limited and they can exhibit a number of undesirable side effects (Goedhard et al., 2006).

Recent evidence in humans and animal models suggests that aggression is highly reinforcing, and aggression in psychiatric patients is associated with structural and functional abnormalities in reward-related brain regions like the nucleus accumbens (NAc) (Couppis and Kennedy, 2008; Decety et al., 2009; Cha et al., 2015; Golden et al., 2016b; Golden et al., 2017). Treatments aimed at reducing the positive valence of aggression through modulation of reward circuitry may be promising options in clinical populations for which elevated aggression is a symptom. The hypothalamic neuropeptide orexin (hypocretin) has been shown to exert modulatory control over reward-related nuclei to critically influence motivation in behavioral paradigms related to feeding, social interaction, and drug seeking (Sakurai, 2014). It is therefore plausible that orexin also influences motivation for engaging in aggression.

Orexin neurons, while relatively few in number, are exclusively located in the lateral hypothalamus (LH) and project throughout the brain. Orexin-A and orexin-B peptides are produced from the precursor pre-pro-orexin and activate target neurons via Gq-coupled orexin receptors orexin receptor 1 (OxR1) and orexin receptor 2 (OxR2) (Kukkonen and Leonard, 2014). While orexin-B binds primarily to OxR2, orexin-A binds to OxR1 and OxR2 with roughly equal affinities (Kukkonen and Leonard, 2014). Until very recently, only two studies had previously investigated the role of orexin in aggressive behavior. One found that orexin knockout mice displayed attenuated physiological defense responses when presented with a conspecific in their home cage (Kayaba and et al., 2003), but levels of aggression in these animals were not directly assessed. Another study, performed in zebrafish, found that pre-pro-orexin mRNA, the precursor for peptides orexin-A and orexin-B, was increased in the LH of dominant males compared to subordinate and control males following aggressive interactions with conspecifics (Pavlidis and et al., 2011). In line with these previous findings, it was very recently found that inhibition of OxR2 expression in the LHb reduced aggressive behavior and conditioned-place preference (CPP) for aggression-paired contexts, indicating that LHb OxR2 signaling is necessary for aggression to be rewarding. Interestingly, it was also found that orexin-A-positive cell bodies in the LH of aggressive mice display increased colocalization with Fos protein compared to non-aggressive mice following the resident intruder (RI) task for territorial aggression in the home cage (Chapter 2). This indicates that orexin neurons projecting to various targets, and not just orexin neurons projecting to the LHb, could be involved in aggression. To broadly test this idea, the brain-penetrant selective OxR2 antagonist N-ethyl-2-[(6-methoxy-pyridin-3-yl)-(toluene-2-sulphonyl)-amino]-N-pyridin-3-ylmethyl-acetamide (EMPA) was systemically administered and aggression and aggression CPP was measured in male mice. It was found that EMPA treatment reduced these behaviors, suggesting that OxR2 antagonism may be effective at reducing aggression in psychiatric patient populations.

Here, it was an aim to further investigate the utility of orexin antagonists to treat aggression by systemically administering drugs targeting either one or both of the two orexin receptors and measuring aggressive behavior in the RI test, anxiety in the open field test, and locomotor behavior. Specifically, it was of interest to compare the anti-aggression effects of the selective OxR2 antagonists EMPA and seltorexant, the selective OxR1 antagonist SR-9659, and the dual orexin receptor antagonist suvorexant. Notably, suvorexant is an FDA-approved treatment for insomnia while seltorexant is in stage 2 clinical trials for the treatment of insomnia. Both drugs appear to be well tolerated by a variety of patient populations with minimal side effects and display low abuse potential, highlighting a positive use profile (Bonaventure and et al., 2018; Yee and et al., 2018). The results of this study indicate that while drugs selectively targeting OxR2 display the highest efficacy in reducing aggression, drugs selectively targeting OxR1 and drugs targeting both orexin receptors are also moderately effective in reducing aggressive behavior.

### Methods

### Animals

4-month old male CD-1 (ICR) mice (RRID: IMSR_CRL:22) (sexually experienced retired breeders; Charles River Laboratories (CRL)) were used as subjects. Subjects were confirmed by CRL to have equal access, experience, and success as breeders. 8-9 week male C57BL/6J mice (RRID: IMSR_JAX:000664) (20-30 g; The Jackson Laboratory) were used as novel intruders. All mice were allowed one week of acclimation to the housing facilities before the start of experiments. Mice were singly housed and C57BL/6J mouse were housed in groups of 5. All mice were maintained on a 12 h light:dark cycle with ad libitum access to food and water. Procedures were performed in accordance with the National Institutes of Health Guide for Care and Use of Laboratory Animals and the Icahn School of Medicine at Mount Sinai Institutional Animal Care and Use Committee.

### Aggression Screening/Resident Intruder (RI) Test

Aggression screening was performed as previously described by utilizing the resident intruder (RI) test (Golden et al., 2016b). After a minimum of one week of habituation to home cages, experimental mice were exposed to a novel C57BL/6J intruder for 3 min daily over 3 consecutive days. Each intruder presentation was performed in the home cage of the experimental mouse between 12-3 PM daily under white light conditions. During RI sessions the cage top was removed to allow for unobstructed viewing and video recording of sessions. The duration and number of screening sessions were selected to prevent induction of stress- and anxiety-related behaviors in experimental mice (Golden et al., 2016b). All RI sessions were video recorded with a digital color video camera. Two blind observers recorded (1) the latency to initial aggression and (2) the total duration of aggression. The initiation of aggression was defined by the first clear physical antagonistic interaction initiated by the resident mouse (usually a bite), not including grooming or pursuit behavior. Aggression was considered completed when the resident mouse had reoriented away from the intruder following the initiation of attack. This definition allows for slight breaks (less than 5 s) in continuous physical interaction within an aggressive bout, assuming the resident mouse has remained oriented towards the intruder throughout. Resident mice were defined as aggressors (AGGs) if they initiated aggression during all three screening sessions, while non-aggressors (NONs) were defined as those that showed no aggression during any screening sessions. Aggression screening was halted if an intruder showed any signs of injury in accordance with the previously published protocol (Golden et al., 2011).

### Drugs

EMPA (30 mg/kg), suvorexant (20 mg/kg), and seltorexant (20 mg/kg) were dissolved in 75% DMSO in sterile water. Vehicle comparison for these drugs consisted of 75% DMSO in sterile water alone. SR-9659 was dissolved in 20% DMSO in sterile water. Vehicle comparison for this drug consisted of 20% DMSO in water alone. All drugs were administered intraperitoneally (I.P.) 30 minutes before behavioral testing. All injection volumes were limited to 100 μl.

### Experimental Timeline

First, mice were screened for three consecutive days in the RI test. Only AGGs were selected for subsequent pharmacology experiments, as it was hypothesized that treatment with orexin antagonists would reduce aggression. One week after screening, AGGs were injected daily with either EMPA, seltorexant, suvorexant, SR-9659, or vehicle and their aggressive behavior was assessed over three consecutive days of the RI test. 24 hours following the last RI test, mice were treated again with either drug or vehicle and anxiety and locomotion were assessed using the open field test.

### Results

### Effects of OxR Antagonists on Aggression

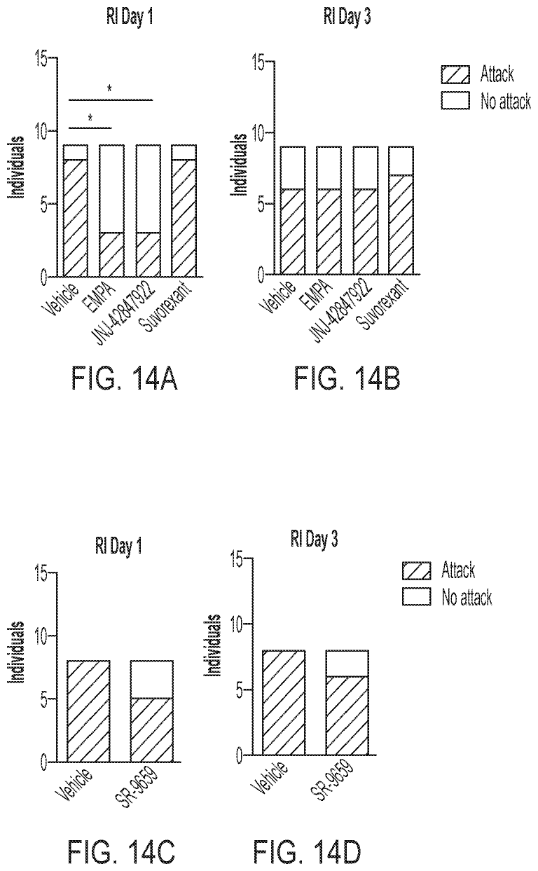

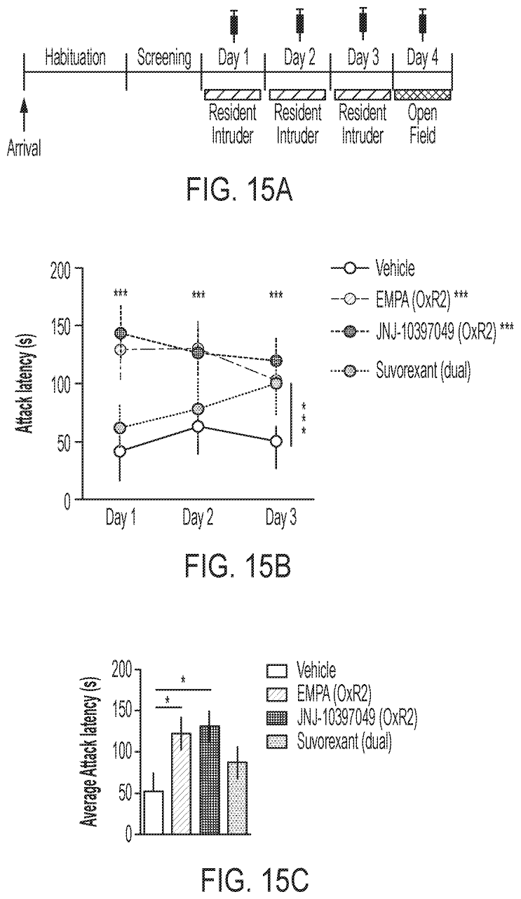

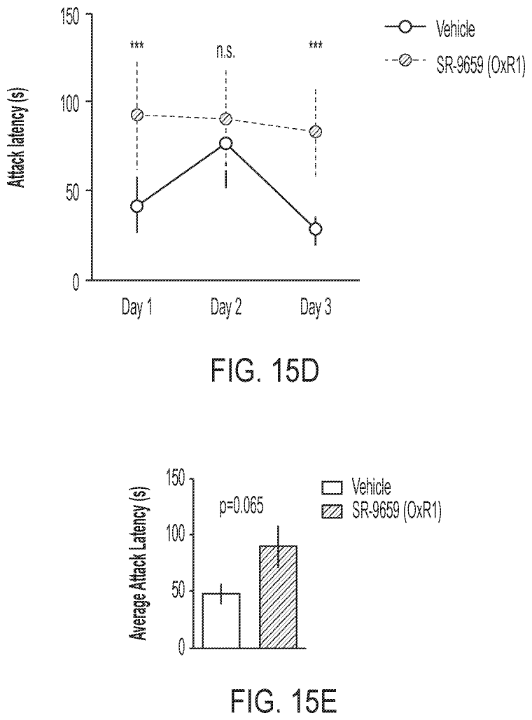

To identify experimental mice exhibiting high levels of aggression, CD-1 male mice were first screened for aggression in the resident intruder (RI) test. In this procedure, resident CD-1 mice are exposed to subordinate male C57BL/6J mice in their home cage for three minutes per day over three consecutive days. Only CD-1 mice that engaged in aggression towards the subordinate conspecific on all three days of screening were selected for experiments. One week following screening, experimental mice were injected daily with either EMPA, seltorexant, suvorexant, SR-9659, or appropriate vehicles and tested in three consecutive days of RI (FIG. 14A). Contingency analysis revealed that on day 1 of RI, mice receiving OxR2 antagonists were less likely to attack conspecifics than mice receiving vehicle (FIGS. 14A-14D). However, this difference was not observed at day 3 of RI, possibly indicating that when aggression becomes highly reinforcing, such as following repeated wins in RI, OxR2 antagonists are less effective at reducing aggression. There were no differences in the number of mice that attacked between suvorexant and vehicle or SR-9659 and vehicle groups on any day of RI. Critically, it was found that treatment with selective OxR2 antagonists EMPA and seltorexant increased the latency to attack and reduced the total duration of attack across all days of RI testing (FIGS. 15A-15E). Suvorexant, however, only altered attack latency and duration on day 3 of RI, indicating that the effects of suvorexant on aggression require chronic treatment. Interestingly, selective OxR1 antagonist SR-9659 was effective in increasing attack latency and reducing duration only on days 1 and 3 of RI, although there was nearly a significant effect of OxR1 antagonism on attack latency overall across the three days of RI (p=0.065) (FIGS. 15A-15E). Together, these data indicate that while all classes of orexin receptor antagonists may have some capacity to reduce aggressive behavior, antagonists selectively targeting OxR2 produce the strongest behavioral effects.

### Effects of OxR Antagonism on Anxiety and Locomotion

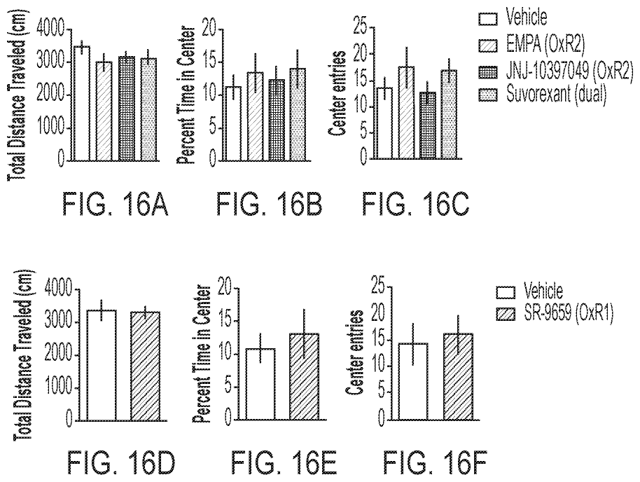

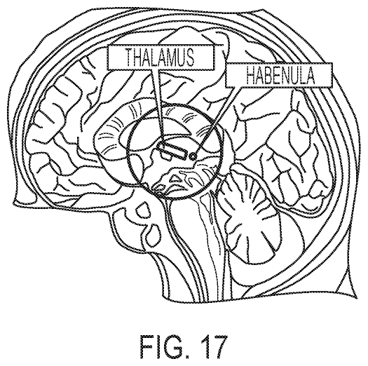

As orexin signaling has been previously implicated in both arousal and anxiety (Tsunematsu et al., 2011; Heydendael and et al., 2014), it was essential that it was verified that the observed effects of orexin receptor antagonism on aggression were not mediated by changes in anxiety or locomotion. To do this, the open field test (OFT) was employed. Roughly 24 hours following the last RI test, mice were treated with orexin antagonists and tested in OFT. There were no observed differences in locomotor or anxiety behavior in mice treated with any of the orexin receptor antagonists (FIGS. 16A-16F). These results indicate that the reductions in aggression observed with orexin receptor antagonist treatment are not attributable to alterations in arousal or anxiety. This lack of arousal or anxiety-related side effects greatly strengthens the feasibility of these drugs for clinical use in aggressive patients.

### Discussion

Here, evidence is provided that pharmacological compounds selectively targeting OxR2 may broadly represent a class of drugs that are effective for the treatment of aggression symptoms. While it was also found that treatment with drugs selectively targeting OxR1 or drugs targeting both OxR1 and OxR2 led to some attenuation of aggressive behavior, these effects were limited and inconsistent across days of testing. Importantly, none of the drugs at the doses tested had any effects on anxiety or locomotion. This indicates that orexin receptor antagonists, in particular OxR2 antagonists, may display marked advantages over current pharmacological treatments for aggression, many of which result in sedation (Goedhard et al., 2006).

Recently, a novel OxR2 microcircuit was identified within the LHb that, when engaged, increased the rewarding properties of aggression and promoted attacks (see Chapter 2). Reduction of OxR2 expression within this circuit attenuated aggression and aggression CPP, indicating that inhibition of OxR2 signaling may be a promising treatment avenue. Because clinical therapeutic options that selectively perturb brain microcircuits are extremely limited, it was tested whether brain-wide antagonism of OxR2 would also reduce aggression and its rewarding properties. Remarkably, acute systemic treatment with 30 mg/kg EMPA reduced aggression CPP and aggression in RI. The present results recapitulate these previous findings while also broadening the scope of possible therapeutic options for targeting OxR2 in aggressive clinical populations. Indeed, the effects of OxR2 antagonism with EMPA versus with seltorexant were virtually indistinguishable from each other over the three days of RI. This not only provides additional evidence supporting the role of OxR2 in aggression, but also suggests that a variety of compounds targeting OxR2 may be effective in treating aggression. It will be important, however, to test whether chronic treatment with OxR2 antagonists can produce long-term reductions in aggressive behavior, particularly when the drug is no longer in circulation. In addition, the remaining known selective OxR2 antagonists (TCS-OX2-29, MK-1064) should be tested for their effects on aggression and aggression valence.

It is believed that this study is the first to test whether treatment with OxR1 or dual orexin receptor antagonists affect aggressive behavior. Although it was found that both the selective OxR1 antagonist SR-9659 and the dual orexin antagonist suvorexant produced some attenuation of aggression, suvorexant's effects appeared to require chronic treatment and SR-9659's effects were inconsistent across multiple days of testing (FIGS. 15A-15E). These results were somewhat surprising considering that 1) numerous previous studies have illustrated that treatment with OxR1, OxR2, and dual orexin receptor antagonists reduces motivation for drugs and drug seeking (Sakurai, 2014; Baimel et al., 2018) and 2) aggressive behavior is highly reinforcing in a manner that behaviorally resembles drug reinforcement (Couppis and Kennedy, 2008; Falkner et al., 2016; Golden et al., 2017). There are a variety of possible explanations for why, in the study, selective antagonism OxR2 was more effective in reducing aggression than selective OxR1 or dual orexin receptor antagonism. First, it is possible that the doses of suvorexant and SR-9659 used here were merely insufficient to elicit reliable reductions in aggression, or that chronic treatment with these drugs was required. On the other hand, it could be that suvorexant and SR-9659 at the doses used here simply reduced motivation for aggression in a manner that was dissociable from the initiation of attacks. Indeed, OxR1 has been more strongly implicated in motivation than OxR2, which conversely has been more strongly implicated in arousal (James et al., 2017). Therefore it is possible that OxR1 controls the motivation for aggression, but not the initiation of it. Future studies should aim to determine if this is the case by testing whether these doses of SR-9659 and suvorexant are sufficient to reduce aggression CPP. Finally, it is possible that the distribution of OxR1 versus OxR2 in the brain differs within nuclei and cell types relevant to aggression such that OxR2, but not OxR1 is necessary for aggression. For example, while both orexin receptors are expressed in regions like the NAc, VTA, and LH, OxR1 expression predominates in the locus coeruleus, the dorsal raphe nucleus, and some amygdala nuclei, and OxR2 expression predominates in the lateral septum, arcuate nucleus of the hypothalamus, and the tuberomammillary nucleus (James and et al., 2018).

Current treatments for aggressive behavior, particularly in psychiatric patients, are few, ineffective, and laden with undesirable side effects. One of these major side effects is sedation, and it is associated with most major accepted pharmacological treatments for aggression (Goedhard et al., 2006). The orexin system has been critically implicated in arousal, and animals lacking orexin peptides display a phenotype similar to that of narcolepsy (Chemelli et al., 1999). Three of the four compounds tested in this study have been shown, at various doses, to promote sleep (Malherbe et al., 2009; Bonaventure and et al., 2018; Yee and et al., 2018), and one, suvorexant (Belsomra®), is a clinically approved treatment for insomnia. Despite the clear indication that antagonism of orexin receptors results in some level of sedation at certain doses, there was no evidence found for this in the study with any of the compounds tested. Importantly, the doses used here were substantially lower (50-66%) than those found to be effective for sleep induction (Malherbe et al., 2009; Bonaventure and et al., 2018; Yee and et al., 2018). It appears, then, that if given at sufficiently low doses, OxR2 antagonists can selectively inhibit aggressive behavior without reducing arousal. While this may become problematic for extremely aggressive individuals who may require large doses of OxR2 antagonists, for some more moderately aggressive patient populations it may be of great clinical utility.

Interestingly, rats bred for high anxiety also exhibit high aggression (Beiderbeck et al., 2012), and some neuropsychiatric disorders associated with aggression are associated with anxiety (Keyes et al., 2016). Activation of orexin neurons has been shown to induce panic in animal models, human patients with anxiety display increased CSF levels of orexin, and inhibition of orexin receptor signaling pharmacologically or genetically reduces anxiety behavior (Johnson and et al., 2010). Despite these indications, there were no reductions in anxiety observed with the doses of orexin antagonists used here. This is again likely due to the low doses of orexin receptor antagonists selected for this study. It is very possible, however, that highly violent individuals requiring larger doses of orexin antagonists for therapeutic reduction of aggression would see concomitant therapeutic reductions in anxiety.

In conclusion, the results suggest that OxR2 antagonists, and to some extent OxR1 and dual orexin receptor antagonists, may be viable treatments for psychiatric patients for which aggression is a symptom. These drugs display marked improvements over current aggression treatments because they can selectively reduce aggression without producing undesirable side effects on arousal or anxiety. It is crucial that additional doses and injection schedules of these drugs are systematically tested in a variety of animal models of aggression in future studies. Moreover, it will be important to determine whether antagonism of orexin receptors at these doses produces adverse effects on measures of motivation or metabolism, which can be dysregulated in psychiatric patients who exhibit aggression (Barlow et al., 2000). If successful in clinical populations without these side effects, the use of orexin antagonists for treatment of aggression could greatly reduce the social and economic burdens of violence.

### REFERENCES (EXAMPLE 2)

Anderson, N. E. and K. A. Kiehl (2014). “Psychopathy and aggression: when paralimbic dysfunction leads to violence.” Curr Top Behav Neurosci 17: 369-393.
Beck, A., A. J. Heinz and A. Heinz (2014). “Translational clinical neuroscience perspectives on the cognitive and neurobiological mechanisms underlying alcohol-related aggression.” Curr Top Behav Neurosci 17: 443-474.
Bonaventure, P., Shelton J., Yun S., Nepomuceno D., Sutton S., Aluisio L., Fraser I., Lord B., Shoblock J., Welty N., Chaplan S. R., Aguilar Z., Halter R., Ndifor A., Koudriakova T., Rizzolio M., Letavic M., Carruthers N. I., Lovenberg T., Dugovic C. (2018). “Characterization of JNJ-42847922, a Selective Orexin-2 Receptor Antagonist, as a Clinical Candidate for the Treatment of Insomnia.—PubMed—NCBI.” J. Pharmacol Exp Ther 354(3): 471-482.
Cha, J., T. Fekete, F. Siciliano, D. Biezonski, L. Greenhill, S. R. Pliszka, J. C. Blader, A. Krain Roy, E. Leibenluft and J. Posner (2015). “Neural Correlates of Aggression in Medication-Naive Children with ADHD: Multivariate Analysis of Morphometry and Tractography.” Neuropsychopharmacology 40(7): 1717-1725.
Coccaro, E. F., D. J. Fridberg, J. R. Fanning, J. E. Grant, A. C. King and R. Lee (2016). “Substance use disorders: Relationship with intermittent explosive disorder and with aggression, anger, and impulsivity.” J Psychiatr Res 81: 127-132.
Couppis, M. H. and C. H. Kennedy (2008). “The rewarding effect of aggression is reduced by nucleus accumbens dopamine receptor antagonism in mice.” Psychopharmacology (Berl) 197(3): 449-456.
Decety, J., K. J. Michalska, Y. Akitsuki and B. B. Lahey (2009). “Atypical empathic responses in adolescents with aggressive conduct disorder: a functional MRI investigation.” Biol Psychol 80(2): 203-211.
Goedhard, L. E., J. J. Stolker, E. R. Heerdink, H. L. Nijman, B. Olivier and T. C. Egberts (2006). “Pharmacotherapy for the treatment of aggressive behavior in general adult psychiatry: A systematic review.” J Clin Psychiatry 67(7): 1013-1024.
Golden, S. A., H. Aleyasin, R. Heins, M. Flanigan, M. Heshmati, A. Takahashi, S. J. Russo and Y. Shaham (2017). “Persistent conditioned place preference to aggression experience in adult male sexually-experienced CD-1 mice.” Genes Brain Behav 16(1): 44-55.
Golden, S. A., C. Heins, M. Venniro, D. Caprioli, M. Zhang, D. H. Epstein and Y. Shaham (2017). “Compulsive Addiction-like Aggressive Behavior in Mice.” Biol Psychiatry 82(4): 239-248.
Golden, S. A., M. Heshmati, M. Flanigan, D. J. Christoffel, K. Guise, M. L. Pfau, H. Aleyasin, C. Menard, H. Zhang, G. E. Hodes, D. Bregman, L. Khibnik, J. Tai, N. Rebusi, B. Krawitz, D. Chaudhury, J. J. Walsh, M. H. Han, M. L. Shapiro and S. J. Russo (2016). “Basal forebrain projections to the lateral habenula modulate aggression reward.” Nature 534(7609): 688-692.
Hoptman, M. J., J. Volavka, P. Czobor, G. Gerig, M. Chakos, J. Blocher, L. L. Citrome, B. Sheitman, J. P. Lindenmayer, J. A. Lieberman and R. M. Bilder (2006). “Aggression and quantitative MRI measures of caudate in patients with chronic schizophrenia or schizoaffective disorder.” J Neuropsychiatry Clin Neurosci 18(4): 509-515.
Kayaba Y., N. A., Kasuya Y., Ohuchi T., Yanagisawa M., Komuro I., Fukada Y., Kuwaki T. (2003). “Attenuated defense response and low basal blood pressure in orexin knockout mice.” Am J Physiol Regul Integr Comp Physiol 285(3): 581-593.
Kukkonen, J. P. and C. S. Leonard (2014). “Orexin/hypocretin receptor signalling cascades.” Br J Pharmacol 171(2): 314-331.
Miles, S. R., D. S. Menefee, J. Wanner, A. Teten Tharp and T. A. Kent (2016). “The Relationship Between Emotion Dysregulation and Impulsive Aggression in Veterans With Posttraumatic Stress Disorder Symptoms.” J Interpers Violence 31(10): 1795-1816.
Pavlidis, M., Sundvik M., Chen Y. C., Panula P. (2011). “Adaptive changes in zebrafish brain in dominant-subordinate behavioral context.” Behav Brain Res 225(2): 529-537.
Sakurai, T. (2014). “The role of orexin in motivated behaviours.” Nat Rev Neurosci 15(11): 719-731.
Sumner, S. A., J. A. Mercy, L. L. Dahlberg, S. D. Hillis, J. Klevens and D. Houry (2015). “Violence in the United States: Status, Challenges, and Opportunities.” JAMA 314(5): 478-488.
Yee, K. L., McCrea J., Panebianco D., Liu W., Lewis N., Cabalu T., Ramael S., Wrishko R. E. (2018). “Safety, Tolerability, and Pharmacokinetics of Suvorexant: A Randomized Rising-Dose Trial in Healthy Men.” Clin Drug Investig 38(7): 631-638.

### Example 3: Systemic Antagonism of Orexin Receptors does not Change Social Recognition, Spatial Memory, or Object Recognition Memory

### Abstract

The effect of orexin receptor antagonists on social recognition, spatial memory, and object memory were assessed compared to controls administered the vehicle alone.

### Methods

### Animals

4-month old male CD-1 (ICR) mice (RRID: IMSR_CRL:22) (sexually experienced retired breeders; Charles River Laboratories (CRL)) were used as subjects. Subjects were confirmed by CRL to have equal access, experience, and success as breeders. 8-9 week male C57BL/6J mice (RRID: IMSR_JAX:000664) (20-30 g; The Jackson Laboratory) were used as novel intruders. All mice were allowed one week of acclimation to the housing facilities before the start of experiments. Mice were singly housed and C57BL/6J mouse were housed in groups of 5. All mice were maintained on a 12 h light:dark cycle with ad libitum access to food and water. Procedures were performed in accordance with the National Institutes of Health Guide for Care and Use of Laboratory Animals and the Icahn School of Medicine at Mount Sinai Institutional Animal Care and Use Committee.

### Drugs

EMPA (30 mg/kg), suvorexant (20 mg/kg), and seltorexant (20 mg/kg) were dissolved in 75% DMSO in sterile water. Vehicle comparison for these drugs consisted of 75% DMSO in sterile water alone. SR-9659 was dissolved in 20% DMSO in sterile water. Vehicle comparison for this drug consisted of 20% DMSO in water alone. All drugs were administered intraperitoneally (IP) 30 minutes before behavioral testing. All injection volumes were limited to 100 μl.

### Recognition and Memory Tests

### Social Recognition Test

Mice were presented with two conspecific mice in a first (training) session, and then one of the two conspecific mice was replaced with an unfamiliar mouse in a second (test) session. The time spent by the test mouse investigating each conspecific mouse provided and index of social memory. This was calculated by subtracting the time spent with the novel mouse from the time spent with the familiar mouse.

### Object Placement Test

Mice were presented with two of the same object in a first (training) session, and then one of the two objects was moved to a different location in the arena during a second (test) session. The time spent investigating each object provided an index of spatial memory. This was calculated by subtracting the time spent with the object in the familiar location from the time spent with the object in the novel location.

### Object Recognition Test

Mice were presented with two different objects in a first (training) session, and then one of the two objects was replaced with a new object during a second (test) session. The time spent investigating each object provided an index of object recognition memory. This was calculated by subtracting the time spent with the familiar object from the time spent with the novel object.

### Results

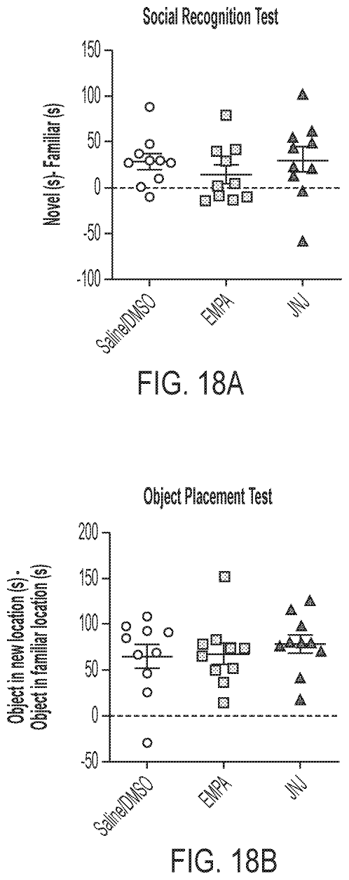

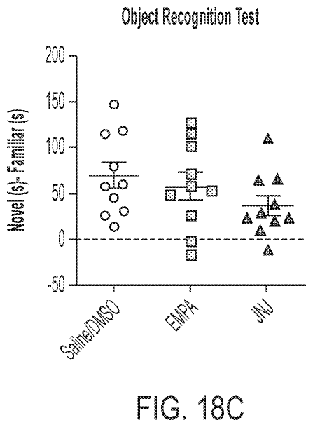

Administration of an orexin receptor antagonist did not impair ability of an animals to distinguish between novel and familiar social targets (i.e., Juvenile mice). Untreated mice spent more time investigating the novel mouse, indicating they recognized the familiar mouse. Treatment with 30 mg/kg EMPA or 20 mg/kg seltorexant (JNJ on graph) did not alter the time spent with each mouse compared to vehicle treatment (one-way ANOVA) (FIG. 18A).

Administration of an orexin receptor antagonist did not affect spatial memory formation. Untreated mice spent more time investigating the object in a new location in the second session, indicating they remember the original placement of the object. Treatment with 30 mg/kg EMPA or 20 mg/kg seltorexant (JNJ on graph) did not alter the time spent with each object compared to vehicle treatment (one-way ANOVA) (FIG. 18B).

Administration of an orexin receptor antagonist did not impair recognition memory when asked to distinguish between a familiar and novel object. Untreated mice spent more time investigating the new object in the second session, indicating they remember the original object. Treatment with 30 mg/kg EMPA or 20 mg/kg seltorexant (JNJ on graph) did not alter the time spent with each object compared to vehicle treatment (one-way ANOVA) (FIG. 18C).

### Discussion

Social recognition, spatial memory, and object recognition tests were performed to assess the effect of orexin receptor antagonists on each respective factor. As described above the data show the administration of an orexin receptor antagonist did not impair the response of the subject mice (FIGS. 18A-18C).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or embodiments of the disclosure, is/are referred to as comprising particular elements and/or features, certain embodiments of the disclosure or embodiments of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A method for treating aggression in a subject comprising administering a therapeutically effective amount of compound I: 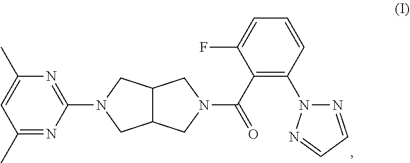 [SMILES: Cc1cc(C)nc(N2CC3CN(C(=O)c4c(F)cccc4-n4nccn4)CC3C2)n1]
or a pharmaceutically acceptable salt thereof.

2. The method of claim 1, wherein the aggression is selected from the group consisting of: Intermittent Explosive Disorder, Kleefstra Syndrome, Impulsive Aggression, X-Linked Intellectual Disability-Hypotonia-Facial Dysmorphism-Aggressive Behavior Syndrome, Gaucher Disease Type 3, Hunters Syndrome (MPS II), and Drug-Induced Aggression.

3. The method of claim 2, wherein the Drug-Induced Aggression is caused by excessive alcohol use, anabolic steroids, benzodiazepines, or cocaine.

4. The method of claim 2, wherein the subject is a human.

5. The method of claim 1, wherein the human subject is addicted to one or more substances that cause Drug-Induced Aggression.

6. The method of claim 1, wherein the subject is an animal.

7. The method of claim 1, wherein the animal is a cat, dog, or horse.

8. The method of claim 1, further comprising the step of selecting a subject having aggression.

9. The method of claim 1, wherein the therapeutically effective amount of compound I does not simultaneously cause sedation or have a cognitive effect compared to a control subject.

10. The method of claim 1, wherein the administration is by an oral, intranasal, transdermal, or intravenous route.

11. A method for treating aggression comprising identifying a subject having aggression, and inhibiting the OX2 receptor.

12. The method according to claim 11, wherein the aggression is associate with a disorder selected from the group consisting of: Intermittent Explosive Disorder, Kleefstra Syndrome, Impulsive Aggression, X-Linked Intellectual Disability-Hypotonia-Facial Dysmorphism-Aggressive Behavior Syndrome, Gaucher Disease Type 3, Hunters Syndrome (MPS II), and Drug-Induced Aggression.

13. The method of claim 12, wherein the Drug-Induced Aggression is caused by excessive alcohol use, anabolic steroids, benzodiazepines, and cocaine.

14. The method of claim 11, wherein the subject is a human.

15. The method of claim 14, wherein the human subject is addicted to one or more substances that cause Drug-Induced Aggression.

16. The method of claim 11, wherein the subject is an animal.

17. The method of claim 16, wherein the animal is a cat, dog, or horse.

18. The method of claim 11, wherein the step of inhibiting the OX2 receptor comprises administering a therapeutically effective amount of an antagonist.

19. The method of claim 18, wherein the antagonist is a nucleic acid that blocks or reduces expression of the OX2 receptor or an agent that specifically binds to the OX2 receptor.

20. The method of claim 19, wherein the agent is a biologic or a small molecule compound.

21. The method of claim 20, wherein the small molecule compound is selected from the group consisting of: suvorexant, almorexant, seltorexant, EMPA, filorexant, JNJ-10397049, and lemborexant.

22. The method of claim 18, wherein the administration is by an oral, intranasal, transdermal, or intravenous route.

23. A method for treating aggression comprising identifying a subject having aggression, and administering a therapeutically effective amount of an OX2 receptor antagonist without having a sedative effect on the subject as compared to a control subject.

24. The method of claim 23, wherein the administering further occurs without an effect on cognition of the subject.

25. The method according to claim 23, wherein the aggression is associated with a disorder selected from the group consisting of: Intermittent Explosive Disorder, Kleefstra Syndrome, Impulsive Aggression, X-Linked Intellectual Disability-Hypotonia-Facial Dysmorphism-Aggressive Behavior Syndrome, Gaucher Disease Type 3, Hunters Syndrome (MPS II), and Drug-Induced Aggression.

26. The method of claim 25, wherein the Drug-Induced Aggression is caused by excessive alcohol use, anabolic steroids, benzodiazepines, and cocaine.

27. The method of claim 23, wherein the subject is a human.

28. The method of claim 27, wherein the human subject is addicted to one or more substances that cause Drug-Induced Aggression.

29. The method of claim 23, wherein the subject is an animal.

30. The method of claim 29, wherein the animal is a cat, dog, or horse.

31. The method of claim 23, wherein the OX2 receptor antagonist is selected from the group consisting of: suvorexant, almorexant, seltorexant, EMPA, filorexant, JNJ-10397049, and lemborexant.

32. A method of treating an animal having aggressive, comprising administering a therapeutically effective amount of an OX2 receptor antagonist without having a sedative effect on the animal as compared to a control subject.

33. The method of claim 32, wherein the animal is a cat, dog, or horse.

34. The method of claim 32, wherein the OX2 receptor antagonist is selected from the group consisting of: suvorexant, almorexant, seltorexant, EMPA, filorexant, JNJ-10397049, and lemborexant.

35. The method of claim 32, wherein OX2 receptor antagonist is incorporated into a food source for feeding the animal.

36. A method for reducing aggression produced as a side-effect of an agent administered to a subject, comprising identifying a subject displaying aggression induced by the agent, and administering a therapeutically effective amount of an OX2 receptor antagonist.

37. The method of claim 36, wherein the agent that produces aggression is a prescription pharmaceutical or biologic.

38. The method of claim 36, wherein the agent that produces aggression is alcohol, an anabolic steroid, a benzodiazepine, or opioid.

39. The method of claim 36, wherein the subject is a human.

40. The method of claim 39, wherein the human subject is addicted to the agent.

41. The method of claim 36, wherein the subject is an animal.

42. The method of claim 41, wherein the animal is a cat, dog, or horse.

43. The method of claim 36, wherein the OX2 receptor antagonist is selected from the group consisting of suvorexant, almorexant, seltorexant, EMPA, filorexant, JNJ-10397049, and lemborexant.

44. The method of claim 36, wherein the administration is by an oral, intranasal, transdermal, or intravenous route.

45. The method of claim 36, wherein the administration of the OX2 receptor antagonist does not impair cognitive function as compared to a control subject.

46. A method for reducing aggressive behavior in a human subject having a mental health disorder, comprising identifying a subject having a mental health disorder who displays an aggressive behavior, and administering a therapeutically effective amount of an OX2 receptor antagonist.

47. The method of claim 46, wherein the mental health disorder is selected from the group consisting of: Alzheimer's disease, attention-deficit/hyperactivity disorder (ADHD), autism spectrum disorder, bipolar disorder, dementia, schizophrenia, and Huntington's disease.

48. The method of claim 46, wherein the OX2 receptor antagonist is selected from the group consisting of: suvorexant, almorexant, seltorexant, EMPA, filorexant, JNJ-10397049, and lemborexant.

49. The method of claim 46, wherein the administration is by an oral, intranasal, transdermal, or intravenous route.

50. The method of claim 46, wherein the administration of the OX2 receptor antagonist does not impair cognitive function as compared to a control subject.
